(19) United States
(12) Patent Application Publication
(10) Pub. No.: US 2002/0077328 A1
(43) Pub. Date: Jun. 20, 2002

(54) Selective cyclooxygenase-2 inhibitors and vasomodulator compounds for generalized pain and headache pain

(76) Inventors: Fred Hassan, Peapack, NJ (US); James C. Forbes, Skokie, IL (US)

Correspondence Address: SENNIGER POWERS LEAVITT AND ROEDEL, ONE METROPOLITAN SQUARE, 16TH FLOOR, ST LOUIS, MO, 63102, US

(21) Appl. No.: 09/905,292

(22) Filed: Jul. 13, 2001

Related U.S. Application Data
(60) Provisional application No. 60/296,196, filed on Jun. 6, 2001.
(60) Provisional application No. 60/284,248, filed on Apr. 17, 2001.
(60) Provisional application No. 60/218,101, filed on Jul. 13, 2000.

(51) Int. Cl. A61K 31/522

(52) U.S. Cl. 514/263.31; 514/263.32

(57) ABSTRACT

A therapeutic combination useful in the treatment, amelioration, prevention, or delay of pain comprising a high energy form of a selective cyclooxygenase-2 inhibitor, a vasomodulator, and a pharmaceutically acceptable excipient, carrier, or diluent, the cyclooxygenase-2 inhibitor and vasomodulator each being present in an amount effective to contribute to the treatment, prevention, ameloriation or delay of pain.

This is a non-provisional application derived from and claiming the benefit of U.S. Provisional Patent Application No. 60/218,101 filed on Jul. 13, 2000, U.S. Provisional Patent Application No. 60/284,248 filed on Apr. 17, 2001, and U.S. Provisional Patent Application No. 60/296,196 filed on Jun. 6, 2001.

### FIELD OF THE INVENTION

The present invention relates to pharmaceutical compositions useful for the treatment, prevention, inhibition, or amelioration of generalized pain and headache pain containing a selective cyclooxygenase-2 inhibitor in combination with a vasomodulator. It also relates to a method of treating generalized pain and headache pain, by administering a selective cyclooxygenase-2 (COX-2) inhibitor in combination with a vasomodulator. In addition, rapid onset formulations of the combination are useful to treat generalized and headache pain due to enhanced bioavailability after administration.

### BACKGROUND OF THE INVENTION

The present invention relates to pharmaceutical compositions of a selective cyclooxygenase-2 inhibitor and a vasomodulator, formulations of the pharmaceutical composition that provide enhanced bioavailability, and a method of using the pharmaceutical composition to treat pain and generalized pain.

The combination of the present invention should provide added efficacy for treating pain, especially headache pain, over current therapies due to the added anti-inflammatory and analgesic properties of the cyclooxygenase-2 and vasomodulator components.

The combination of the invention can be used prophylactically or for treatment of acute pain. The rapid onset formulations of the invention are particularly useful to treat an acute attack due to their enhanced bioavailability and shortened time to reach a threshold therapeutic concentration.

Selective inhibition of COX-2 has been shown to have anti-inflammatory and analgesic properties without the associated gastric and kidney related toxicity problems. This phenomenon is due to the discovery of NSAIDs that are capable of inhibiting COX-2, which is responsible for the production of prostaglandins that mediate the inflammatory response, without causing the inhibition of COX-1, which is responsible for the production of prostaglandins that maintain both gastrointestinal integrity, and kidney function. Thus, the beneficial effects of NSAIDs are separable from their drastic side effects by the development of COX-2 selective inhibitors.

Toward that end, several drugs that are COX-2 selective inhibitors of prostaglandin synthesis have been developed. The most extensively characterized class of COX-2 selective inhibitor is diarylheterocycles, which include the recently approved drugs celecoxib and rofecoxib. However, other classes include, but are not limited to, acidic sulfonamides, indomethacin analogs, zomepirac analogs, chromene analogs and di-t-butylphenols. For example, U.S. Pat. No. 5,380,738 describes oxazoles which selectively inhibit COX-2, U.S. Pat. No. 5,344,991 describes cyclopentenes which selectively inhibit COX-2, U.S. Pat. No. 5,393,790 describes spiro compounds which selectively inhibit COX-2, WO94/15932 describes thiophene and furan derivatives which selectively inhibit COX-2, and WO95/15316 describes pyrazolyl sulfonamide derivatives which selectively inhibit COX-2.

In addition, vasomodulators can affect the physiological origins of generalized and headache pain. In particular, caffeine is known to have analgesic properties useful to treat generalized and headache pain as well as other conditions.

Australian Patent Applications No. 200042711, No. 200043730 and No. 200043736 disclose compositions comprising a selective COX-2 inhibitory drug, a 5HT₁ receptor agonist and caffeine, said to be useful for treating migraine.

A need for formulated compositions of selective COX-2 inhibitory drugs, particularly rapid-onset compositions of such drugs, exists. Rapid-onset drug delivery systems can provide many benefits over conventional dosage forms. Generally, rapid-onset preparations provide a more immediate therapeutic effect than standard dosage forms. For example, in the treatment of acute pain, for example in headache or migraine, rapid-onset dosage forms would be useful to provide fast pain relief.

### SUMMARY OF THE INVENTION

### BRIEF DESCRIPTION OF THE DRAWINGS

FIG. 1 shows the blood plasma concentrations of two formulations of celecoxib, F1 and a solid capsule formulation, after administration to dogs. The composition of the F1 formulation is shown in Table 13 herein.

FIG. 2 shows the blood plasma concentrations of two formulations of celecoxib, F3 and a solid capsule formulation, after administration to dogs. The composition of the F3 formulation is shown in Table 13 herein.

FIG. 3 shows the blood plasma concentrations of two formulations of celecoxib, F4 and a solid capsule formulation, after administration to dogs. The composition of the F4 formulation is shown in Table 13 herein.

FIG. 4 shows the in vitro dissolution profiles of five formulations: Fl, F3, F4, F5 and F7. Compositions of these formulations are described in Table 13 herein.

FIG. 5 shows the in vitro dissolution profiles of three formulations: F8, F9 and F10. Compositions of these formulations are described in Table 8 herein.

FIG. 6 shows a powder X-ray diffraction profile of a celecoxib drug substance C1 prepared in Example 11, by comparison with crystalline celecoxib C2.

FIG. 7 shows powder X-ray diffraction profiles of a celecoxib-polymer composite C3 of the invention immediately after preparation (T1) and following storage for 2 weeks at 40° C. and 75% relative humidity (T2).

FIG. 8 shows powder X-ray diffraction profiles of a celecoxib-polymer composite C4 of the invention immediately after preparation (T1) and following storage for 2 weeks at 40° C. and 75% relative humidity (T2).

FIG. 9 shows a differential scanning calorimetry (DSC) thermogram of a celecoxib drug substance C1 comprising no polymer.

FIG. 10 shows a DSC thermogram of a celecoxib-polymer composite C3 of the invention wherein the polymer is hydroxypropylmethylcellulose.

FIG. 11 shows a DSC thermogram of a celecoxib-polymer composite C4 of the invention wherein the polymer is polyvinylpyrrolidone.

FIG. 12 shows blood plasma concentration profiles of celecoxib administered as a single oral dose of 200 mg, in the form of a capsule (Celebrex® 200 mg, Pharmacia Corporation) or in the form of a suspension in apple juice as described herein.

FIG. 13 shows relief of post-surgical pain experienced over a 12-hour period following administration of a single oral dose of (1) 200 mg celecoxib in the form of a capsule (Celebrex® 200 mg, Pharmacia Corporation), (2) 400 mg ibuprofen in the form of a capsule, (3) 200 mg celecoxib in the form of a fine suspension in apple juice as described herein, or (4) placebo.

FIG. 14 shows more clearly than FIG. 13 the relief of post-surgical pain experienced in the first 2 hours following administration of the above treatments (1) through (4), to emphasize differences among treatments in time of onset of pain relief.

FIG. 15 is a flow diagram illustrating a representative method for preparation of valdecoxib tablets of the invention.

FIG. 16 is a flow diagram illustrating an alternative method for preparation of valdecoxib tablets of the invention.

FIG. 17 is a graph showing plasma concentration of valdecoxib in dogs following oral administration of valdecoxib tablets of the invention.

FIG. 18 is a graph showing plasma concentration of valdecoxib in humans following oral administration of valdecoxib tablets of the invention.

### DETAILED DESCRIPTION OF THE INVENTION

Compositions comprising selective cyclooxygenase-2 (COX-2) inhibitor compounds and vasomodulators are particularly beneficial to alleviate generalized or headache pain. Selective cyclooxygenase-2 inhibitors effectively control the cyclooxygenase-2 mediated production of prostaglandins in response to injury or inflammation. The inhibition of COX-1 mediated constitutive functions affecting renal and gastrointestinal physiology are reduced by using cyclooxygenase-2 inhibitors because selective cyclooxygenase-2 inhibitors preferentially inhibit cyclooxygenase-2 mediated physiological pathways. Thus, selective cyclooxygenase-2 inhibitors should be safer than non-selective cyclooxygenase inhibitors such as non-steroidal anti-inflammatory drugs (NSAIDS) because the inhibition of cyclooxygenase-1 is reduced and the effects on the renal and gastrointestinal systems should be reduced accordingly.

In addition, vasomodulators are known to affect the mechanisms giving rise to pain, especially headache pain. Under the vasogenic theory, intracranial vasoconstriction was responsible for the symptoms of migraine aura and headache resulted from a rebound dilation and distention of cranial vessels and activation of perivascular nociceptive axons. However, under the alternate nerogenic theory, the brain generates the migraine and susceptibility to migraine attacks reflects thresholds intrinsic to the individual's brain. Thus, vascular changes occurring during migraine are the result and not the cause of the attack. Even considering the alternate theories of migraine, vascular changes are implicated as an important event during the headache. Thus, using a vasomodulator to affect vascular changes in addition to a cyclooxygenase-2 inhibitory compound to inhibit cyclooxygenase-2 mediated prostaglandin synthesis has a beneficial effect on generalized and headache pain.

Methylated xanthines such as caffeine, theophylline, and theobromine, and derivatives thereof, have many common pharmacological actions. They relax smooth muscle, stimulate the central nervous system, stimulate cardiac muscle, and act on the kidney as a diuretic.

The present disclosure provides pharmaceutical compositions of a selective cyclooxygenase-2 inhibitor in combination with a vasomodulator. The combination of the present invention may be administered via a rapid-onset vehicle. In addition, the disclosure provides a method of treatment for generalized and headache pain using the pharmaceutical compositions of the invention.

Another embodiment of the present invention is a pharmaceutical composition for the combination of a methylxanthine compound, or other bronchodilator, preferably caffeine, xanthine, theophylline, or theobromine, and a selective cyclooxygenase-2 inhibitor, for the treatment of generalized pain or headache pain, comprising a therapeutically-effective amount of a selective cyclooxygenase-2 inhibitor and a methylxanthine or other bronchodilator. The combination of the invention can be in association with at least one pharmaceutically-acceptable carrier, adjuvant or diluent (collectively referred to herein as “carrier” materials) and, if desired, other active ingredients. The active compounds of the present invention may be administered by any suitable route known to those skilled in the art. For example, they can be administered orally, intravascularly, intraperitoneally, intranasal, intrabronchial, subcutaneously, intramuscularly, parenterally, rectally, or topically (including aerosol). If the pain is localized, local administration rather than system administration may be used. Formulation in a lipid vehicle may be used to enhance bioavailability.

The administration of the present invention may be for either prevention or treatment purposes. The methods and compositions used herein may be used alone or in conjunction with additional therapies known to those skilled in the art in the prevention or treatment of pain, inflammation, or arthritis. Alternatively, the methods and compositions described herein may be used as adjunct therapy.

I. COX-2 Inhibitory Compounds Used in the Invention

The following cyclooxygenase-2 inhibitors are included in the practice of this invention.

The combination and method provided herein relates to the use of cyclooxygenase-2 selective inhibitors or prodrugs or pharmaceutically acceptable salts thereof in combination with a vasomodulator for the prevention or treatment of generalized or headache pain. In one embodiment, the cyclooxygenase-2 selective inhibitor can be, for example, the COX-2 selective inhibitor meloxicam, Formula B-1 (CAS registry number 71125-38-7) or a pharmaceutically acceptable salt or prodrug thereof.

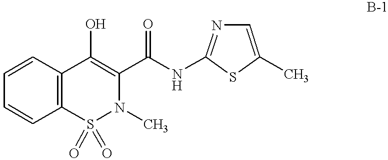

In yet another embodiment, the cyclooxygenase-2 selective inhibitor is the COX-2 selective inhibitor, 6-[[5-(4-chlorobenzoyl)-1,4-dimethyl-1H-pyrrol-2-yl]methyl]-3(2H)-pyridazinone, Formula B-2 (CAS registry number 179382-91-3) or a pharmaceutically acceptable salt or prodrug thereof.

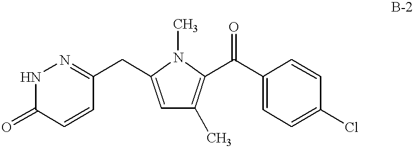

In a preferred embodiment the cyclooxygenase-2 selective inhibitor is preferably of the chromene structural class that is a substituted benzopyran or a substituted benzopyran analog, and even more preferably selected from the group consisting of substituted benzothiopyrans, dihydroquinolines, or dihydronaphthalenes having the general Formula I shown below and possessing, by way of example and not limitation, the structures disclosed in Table 1, including the diastereomers, enantiomers, racemates, tautomers, salts, esters, amides and prodrugs thereof. Furthermore, benzopyran COX-2 selective inhibitors useful in the practice of the present methods are described in U.S. Pat. Nos. 6,034,256 and 6,077,850 herein incorporated by reference.

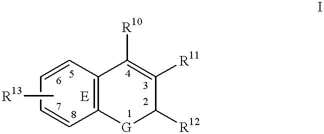

wherein G is selected from the group consisting of O or S or NR^{a}; wherein R^{a} is alkyl;

wherein R¹⁰ is selected from the group consisting of H and aryl

wherein R¹¹ is selected from the group consisting of carboxyl, aminocarbonyl, alkylsulfonylaminocarbonyl and alkoxycarbonyl;

wherein R¹² is selected from the group consisting of haloalkyl, alkyl, aralkyl, cycloalkyl and aryl optionally substitutued with one or more radicals selected from alkylthio, nitro and alkylsulfonyl; and

wherein R¹³ is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkyl amino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R¹³ together with ring E forms a naphthyl radical;

or a pharmaceutically acceptable salt or isomer or prodrug thereof.

In a further embodiment, the cyclooxygenase-2 selective inhibitor comprises a compound of formula I wherein:

G is selected from the group consisting of oxygen and sulfur;

R¹¹ is selected from the group consisting of carboxyl, lower alkyl, lower aralkyl and lower alkoxycarbonyl;

R¹² is selected from the group consisting of lower haloalkyl, lower cycloalkyl and phenyl; and

R¹³ is one or more radicals selected from the group consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl lower aralkylaminosulfonyl, 5-membered nitrogen containing heterocyclosulfonyl, 6-membered nitrogen containing heterocyclosulfonyl lower alkylsulfonyl, optionally substituted phenyl, lower aralkylcarbonyl, and lower alkylcarbonyl; or wherein R¹³ together with ring E forms a naphthyl radical;

or a pharmaceutically acceptable salt, or isomer, or prodrug thereof.

In still another embodiment, the cyclooxygenase-2 selective inhibitor comprises a compound of formula I wherein:

R¹¹ is carboxyl;

R¹² is lower haloalkyl; and

R¹³ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen containing heterocyclosulfonyl, optionally substituted phenyl, lower aralkylcarbonyl, and lower alkylcarbonyl; or wherein R¹³ together with ring E forms a naphthyl radical; or a pharmaceutically acceptable salt, or isomer, or prodrug thereof.

In a yet a further embodiment, the cyclooxygenase-2 selective inhibitor comprises a compound of formula I wherein:

R¹² is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and

R¹³ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R¹³ together with ring E forms a naphthyl radical;

or a pharmaceutically acceptable salt, or isomer, or prodrug thereof.

In another embodiment, the cyclooxygenase-2 selective inhibitor comprises a compound of formula I wherein:

R¹² is selected from the group consisting of trifluoromethyl and pentafluorethyl; and

R¹³ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R¹³ together with ring E forms a naphthyl radical;

or a pharmaceutically acceptable salt, or isomer, or prodrug thereof.

Exemplary compounds which are useful in the present method include, but are not limited to:

6-chloro-2-trifluoromethyl-2H-1-benzopyran-3-carboxylic acid;

6-chloro-7-methyl-2-trifluoromethyl-2H-1-benzopyran-3-carboxylic acid;

8-(1-methylethyl)-2-trifluoromethyl-2H-1-benzopyran-3-carboxylic acid;

6-chloro-7-(1,1-dimethylethyl)-2-trifluoromethyl-2H-1-benzopyran-3-carboxylic acid;

6-chloro-8-(1-methylethyl)-2-trifluoromethyl-2H-1-benzopyran-3-carboxylic acid;

2-trifluoromethyl-3H-naphthopyran-3-carboxylic acid

7-(1,1-dimethylethyl)-2-trifluoromethyl-2H-1-benzopyran-3-carboxylic acid;

6-bromo-2-trifluoromethyl-2H-1-benzopyran-3-carboxylic acid;

8-chloro-2-trifluoromethyl-2H-1-benzopyran-3-carboxylic acid;

6-trifluoromethoxy-2-trifluoromethyl-2H-1-benzopyran-3-carboxylic acid;

5,7-dichloro-2-trifluoromethyl-2H-1-benzopyran-3-carboxylic acid;

8-phenyl-2-trifluoromethyl-2H-1-benzopyran-3-carboxylic acid;

7,8-dimethyl-2-trifluoromethyl-2H-1-benzopyran-3-carboxylic acid;

6,8-bis(dimethylethyl)-2-trifluoromethyl-2H-1-benzopyran-3-carboxylic acid;

7-(1-methylethyl)-2-trifluoromethyl-2H-1-benzopyran-3-carboxylic acid;

7-phenyl-2-trifluoromethyl-2H-1-benzopyran-3-carboxylic acid;

6-chloro-7-ethyl-2-trifluoromethyl-2H-1-benzopyran-3-carboxylic acid;

6-chloro-8-ethyl-2-trifluoromethyl-2H-1-benzopyran-3-carboxylic acid;

6-chloro-7-phenyl-2-trifluoromethyl-2H-1-benzopyran-3-carboxylic acid;

6,7-dichloro-2-trifluoromethyl-2H-1-benzopyran-3-carboxylic acid;

6,8-dichloro-2-trifluoromethyl-2H-1-benzopyran-3-carboxylic acid;

2-trifluoromethyl-3H-naptho[2,1-b]pyran-3-carboxylic acid;

6-chloro-8-methyl-2-trifluoromethyl-2H-1-benzopyran-3-carboxylic acid;

8-chloro-6-methyl-2-trifluoromethyl-2H-1-benzopyran-3-carboxylic acid;

8-chloro-6-methoxy-2-trifluoromethyl-2H-1-benzopyran-3-carboxylic acid;

6-bromo-8-chloro-2-trifluoromethyl-2H-1-benzopyran-3-carboxylic acid;

8-bromo-6-fluoro-2-trifluoromethyl-2H-1-benzopyran-3-carboxylic acid;

8-bromo-6-methyl-2-trifluoromethyl-2H-1-benzopyran-3-carboxylic acid;

8-bromo-5-fluoro-2-trifluoromethyl-2H-1-benzopyran-3-carboxylic acid;

6-chloro-8-fluoro-2-trifluoromethyl-2H-1-benzopyran-3-carboxylic acid;

6-bromo-8-methoxy-2-trifluoromethyl-2H-1-benzopyran-3-carboxylic acid;

6-[[(phenylmethyl)amino]sulfonyl]-2-trifluoromethyl-2H-1-benzopyran-3-carboxylic acid;

6-[(dimethylamino)sulfonyl]-2-trifluoromethyl-2H-1-benzopyran-3-carboxylic acid;

6-[(methylamino)sulfonyl]-2-trifluoromethyl-2H-1-benzopyran-3-carboxylic acid;

6-[(4-morpholino)sulfonyl]-2-trifluoromethyl-2H-1-benzopyran-3-carboxylic acid;

6-[(1,1-dimethylethyl)aminosulfonyl]-2-trifluoromethyl-2H-1-benzopyran-3-carboxylic acid;

6-[(2-methylpropyl)aminosulfonyl]-2-trifluoromethyl-2H-1-benzopyran-3-carboxylic acid;

6-methylsulfonyl-2-trifluoromethyl-2H-1-benzopyran-3-carboxylic acid;

8-chloro-6-[[(phenylmethyl)amino]sulfonyl]-2-trifluoromethyl-2H-1-benzopyran-3-carboxylic acid;

6-phenylacetyl-2-trifluoromethyl-2H-1-benzopyran-3-carboxylic acid;

6,8-dibromo-2-trifluoromethyl-2H-1-benzopyran-3-carboxylic acid;

8-chloro-5,6-dimethyl-2-trifluoromethyl-2H-1-benzopyran-3-carboxylic acid;

6,8-dichloro-(S)-2-trifluoromethyl-2H-1-benzopyran-3-carboxylic acid;

6-benzylsulfonyl-2-trifluoromethyl-2H-1-benzopyran-3-carboxylic acid;

6-[[N-(2-furylmethyl)amino]sulfonyl]-2-trifluoromethyl-2H-1-benzopyran-3-carboxylic acid;

6-[[N-(2-phenylethyl)amino]sulfonyl]-2-trifluoromethyl-2H-1-benzopyran-3-carboxylic acid;

6-iodo-2-trifluoromethyl-2H-1-benzopyran-3-carboxylic acid;

7-(1,1-dimethylethyl)-2-pentafluoroethyl-2H-1-benzopyran-3-carboxylic acid; and

6-chloro-2-trifluoromethyl-2H-1-benzothiopyran-3-carboxylic acid.

TABLE 1
Examples of Chromene COX-2 Selective Inhibitors as Embodiments

| Compound | |
|---|---|
| Number | Structural Formula |
| B-3 | 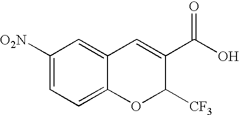 |
| | 6-Nitro-2-trifluoromethyl-2H-1- |
| | benzopyran-3-carboxylic acid |
| B-4 | 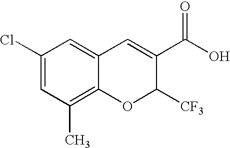 |
| | 6-Chloro-8-methyl-2-trifluoromethyl- |
| | 2H-1-benzopyran-2-carboxylic acid |
| B-5 | 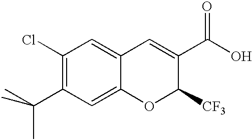 |
| | ((S)-6-Chloro-7-(1,1-dimethylethyl)-2- |
| | (trifluoromethyl-2H-1-benzopyran-3-carboxylic acid |
| B-6 | 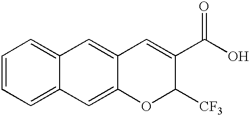 |
| | 2-Trifluoromethyl-2H-naphtho[2,3-b] |
| | pyran-3-carboxylic acid |
| B-7 | 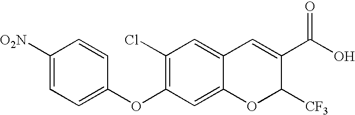 |
| | 6-Chloro-7-(4-nitrophenoxy)-2-(trifluoromethyl)- |
| | 2H-1-benzopyran-3-carboxylic acid |
| B-8 | 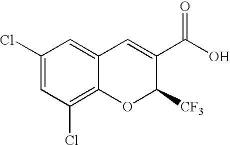 |
| | ((S)-6,8-Dichloro-2-(trifluoromethyl)- |
| | 2H-1-benzopyran-3-carboxylic acid |
| B-9 | 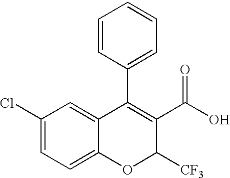 |
| | 6-Chloro-2-(trifluoromethyl)-4-phenyl-2H- |
| | 1-benzopyran-3-carboxylic acid |
| B-10 | 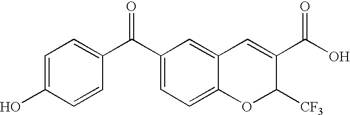 |
| | 6-(4-Hydroxybenzoyl)-2-(trifluoromethyl)- |
| | 2H-1-benzopyran-3-carboxylic acid |
| B-11 | 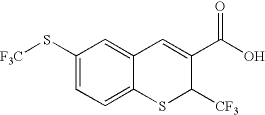 |
| | 2-(Trifluoromethyl)-6-[(trifluoromethyl)thio]- |
| | 2H-1-benzothiopyran-3-carboxylic acid |
| B-12 | 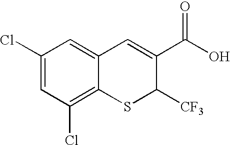 |
| | 6,8-Dichloro-2-trifluoromethyl-2H-1- |
| | benzothiopyran-3-carboxylic acid |
| B-13 | 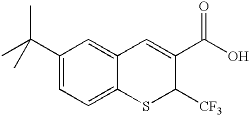 |
| | 6-(1,1-Dimethylethyl)-2-(trifluoromethyl)- |
| | 2H-1-benzothiopyran-3-carboxylic acid |
| B-14 | 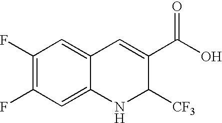 |
| | 6,7-Difluoro-1,2-dihydro-2-(trifluoromethyl)- |
| | 3-quinolinecarboxylic acid |
| B-15 | 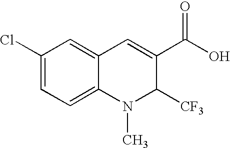 |
| | 6-Chloro-1,2-dihydro-1-methyl-2-(trifluoro |
| | methyl)-3-quinolinecarboxylic acid |
| B-16 | 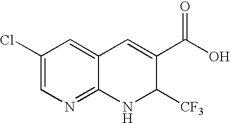 |
| | 6-Chloro-2-(trifluoromethyl)-1,2-dihydro |
| | [1,8]naphthyridine-3-carboxylic acid |
| B-17 | 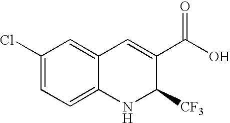 |
| | ((S)-6-Chloro-1,2-dihydro-2-(trifluoromethyl)- |
| | 3-quinolinecarboxylic acid |

In a further preferred embodiment, the cyclooxygenase inhibitor is selected from the class of tricyclic cyclooxygenase-2 selective inhibitors represented by the general structure of Formula II:

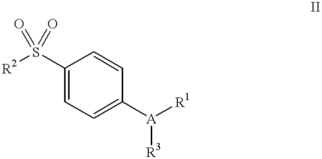

wherein A is selected from the group consisting of partially unsaturated or unsaturated heterocyclyl and partially unsaturated or unsaturated carbocyclic rings;

wherein R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is selected from the group consisting of methyl or amino; and

wherein R³ is selected from the group consisting of a radical selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt thereof.

In a still more preferred embodiment of the invention the cyclooxygenase-2 selective inhibitor represented by the above Formula II is selected from the group of compounds, illustrated in Table 2, consisting of celecoxib (B-18; U.S. Pat. No. 5,466,823; CAS No. 169590-42-5), valdecoxib (B-19; U.S. Pat. No. 5,633,272; CAS No. 181695-72-7), deracoxib (B-20; U.S. Pat. No. 5,521,207; CAS No. 169590-41-4), rofecoxib (B-21; CAS No. 162011-90-7), etoricoxib (MK-663; B-22; PCT publication WO 98/03484), JTE-522 (B-23), or a pharmaceutically acceptable salt or prodrug thereof.

TABLE 2
Examples of Tricyclic COX-2 Selective Inhibitors as Embodiments

| Compound | |
|---|---|
| Number | Structural Formula |
| B-18 | 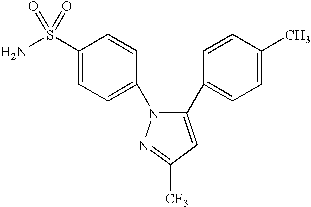 |
| B-19 | 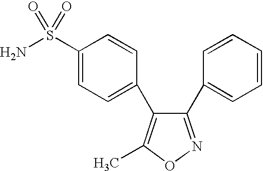 |
| B-20 | 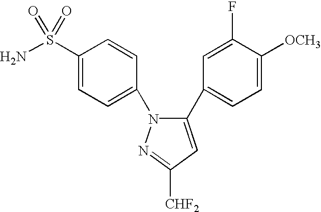 |
| B-21 | 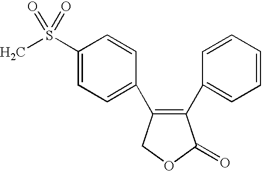 |
| B-22 | 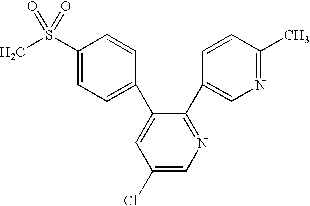 |
| B-23 | 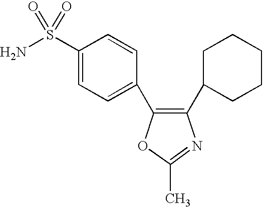 |

In an even more preferred embodiment, the COX-2 selective inhibitor is selected from the group consisting of celecoxib, rofecoxib and etoricoxib.

In another highly preferred embodiment of the invention, parecoxib (B-24, U.S. Pat. No. 5,932, 598, CAS No. 198470-84-7), which is a therapeutically effective prodrug of the tricyclic cyclooxygenase-2 selective inhibitor valdecoxib, B-19, may be advantageously employed as a source of a cyclooxygenase inhibitor (U.S. Pat. No. 5,932,598, herein incorporated by reference).

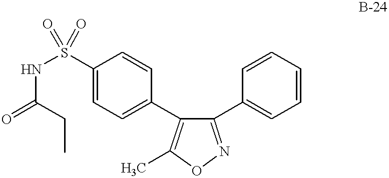

In another preferred embodiment of the invention, the compound having the formula B-25 that has been previously described in International Publication number WO 00/24719 (which is herein incorporated by reference), is another tricyclic cyclooxygenase-2 selective inhibitor which may be advantageously employed.

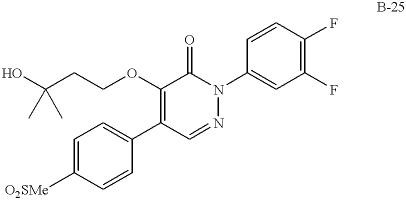

Other compounds which may advantageously employed include 4-[4-(methyl)-sulfonyl)phenyl]-3-phenyl-2(5H)-furanone, 4-(5-methyl-3-phenyl-4-isoxazolyl), 2-(6-methylpyrid-3-yl)-3-(4-methylsulfonylphenyl)-5-chloropyridine, 4-[5-(4-methylphenyl)-3-(trifluoromethyl)-1H-pyrazol-1-yl], N-[[4-(5-methyl-3-phenyl-4-isoxazolyl)phenyl]sulfonyl], 4-[5-(3-fluoro-4-methoxyphenyl)-3-difluoromethyl)-1H-pyrazol-1-yl]benzenesulfonamide, (S)-6,8-dichloro-2-(trifluoromethyl)-2H-1-benzopyran-3-carboxylic acid, and 2-(3,4-difluorophenyl)-4-(3-hydroxy-3-methylbutoxy)-5-[4-(methylsulfonyl)phenyl]-3(2H)-pyridzainone.

In another embodiment, the cyclooxygenase-2 selective inhibitor comprises a compound of the formula;

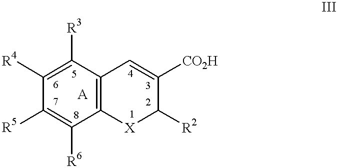

wherein X is O or S;

R² is lower haloalkyl;

R³ is selected from the group consisting of hydrido and halo;

R⁴ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, a 5-membered nitrogen containing heterocyclosulfonyl, and a 6-membered nitrogen containing heterocyclosulfonyl;

R⁵ is selected from the group consisting of hydrido, lower alkyl, halo, lower alkoxy, and aryl; and

R⁶ is selected from the group consisting of hydrido, halo, lower alkyl, lower alkoxy, and aryl. or a pharmaceutically acceptable salt, or isomer, or prodrug thereof.

In another embodiment, the cyclooxygenase-2 selective inhibitor comprises a compound of formula III wherein:

R² is selected from the group consisting of trifluoromethyl and pentafluoroethyl;

R³is selected from the group consisting of hydrido, chloro, and fluoro;

R⁴ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;

R⁵ is selected from the group consisting of hydrido, methyl, ethyl, isopropyl, tert-butyl, chloro, methoxy, diethylamino, and phenyl; and

R⁶ is selected from the group consisting of hydrido, chloro, bromo, fluoro, methyl, ethyl, tert-butyl, methoxy, and phenyl;

or a pharmaceutically acceptable salt, or isomer, or prodrug thereof.

In another preferred embodiment of the invention, the selective COX-2 inhibitor comprises a compound of formula IV as described in International Publication number WO 99/11605 (which is herein incorporated by reference);

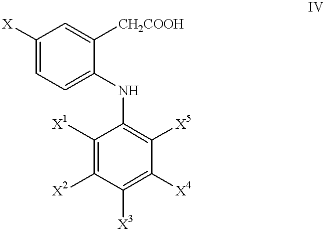

wherein X is methyl or ethyl;

X¹ is chloro or fluoro;

X² is hydrido or fluoro;

X³ is hydrido, fluoro, chloro, methyl, ethyl, methoxy, ethoxy, or hydroxy;

X⁴ is hydrido or fluoro; and

X⁵ is chloro, fluoro, trifluoromethyl or methyl;

pharmaceutically acceptable salts thereof; and

pharmaceutically acceptable prodrug esters thereof.

The process for preparing the compounds of Formula IV, above, are detailed in International Publication Number SO 01/23346, which is herein incorporated by reference.

Any such selective COX-2 inhibitory drug known in the art can be used, including without limitation compounds disclosed in the Patents and publications listed below, each of which is individually incorporated herein by reference.

U.S. Pat. No. 5,344,991 to Reitz & Li.

U.S. Pat. No. 5,380,738 to Norman et al.

U.S. Pat. No. 5,393,790 to Reitz et al.

U.S. Pat. No. 5,401,765 to Lee.

U.S. Pat. No. 5,418,254 to Huang & Reitz.

U.S. Pat. No. 5,420,343 to Koszyk & Weier.

U.S. Pat. No. 5,434,178 to Talley & Rogier.

U.S. Pat. No. 5,436,265 to Black et al.

Above-cited U.S. Pat. No. 5,466,823.

U.S. Pat. No. 5,474,995 to Ducharme et al.

U.S. Pat. No. 5,475,018 to Lee & Bertenshaw.

U.S. Pat. No. 5,486,534 to Lee et al.

U.S. Pat. No. 5,510,368 to Lau et al.

U.S. Pat. No. 5,521,213 to Prasit et al.

U.S. Pat. No. 5,536,752 to Ducharme et al.

U.S. Pat. No. 5,543,297 to Cromlish et al.

U.S. Pat. No. 5,547,975 to Talley et al.

U.S. Pat. No. 5,550,142 to Ducharme et al.

U.S. Pat. No. 5,552,422 to Gauthier et al.

U.S. Pat. No. 5,585,504 to Desmond et al.

U.S. Pat. No. 5,593,992 to Adams et al.

U.S. Pat. No. 5,596,008 to Lee.

U.S. Pat. No. 5,604,253 to Lau et al.

U.S. Pat. No. 5,604,260 to Guay & Li.

U.S. Pat. No. 5,616,458 to Lipsky et al.

U.S. Pat. No. 5,616,601 to Khanna et al.

U.S. Pat. No. 5,620,999 to Weier et al.

Above-cited U.S. Pat. No. 5,633,272.

U.S. Pat. No. 5,639,780 to Lau et al.

U.S. Pat. No. 5,643,933 to Talley et al.

U.S. Pat. No. 5,658,903 to Adams et al.

U.S. Pat. No. 5,668,161 to Talley et al.

U.S. Pat. No. 5,670,510 to Huang & Reitz.

U.S. Pat. No. 5,677,318 to Lau.

U.S. Pat. No. 5,681,842 to Dellaria & Gane.

U.S. Pat. No. 5,686,460 to Nicolal et al.

U.S. Pat. No. 5,686,470 to Weier et al.

U.S. Pat. No. 5,696,143 to Talley et al.

U.S. Pat. No. 5,710,140 to Ducharme et al.

U.S. Pat. No. 5,716,955 to Adams et al.

U.S. Pat. No. 5,723,485 to Güngör & Teulon.

U.S. Pat. No. 5,739,166 to Reitz et al.

U.S. Pat. No. 5,741,798 to Lazer et al.

U.S. Pat. No. 5,756,499 to Adams et al.

U.S. Pat. No. 5,756,529 to Isakson & Talley.

U.S. Pat. No. 5,776,967 to Kreft et al.

U.S. Pat. No. 5,783,597 to Beers & Wachter.

U.S. Pat. No. 5,789,413 to Black et al.

U.S. Pat. No. 5,807,873 to Nicolai & Teulon.

U.S. Pat. No. 5,817,700 to Dube et al.

U.S. Pat. No. 5,830,911 to Failli et al.

U.S. Pat. No. 5,849,943 to Atkinson & Wang.

U.S. Pat. No. 5,859,036 to Sartori et al.

U.S. Pat. No. 5,861,419 to Dube et al.

U.S. Pat. No. 5,866,596 to Sartori & Teulon.

U.S. Pat. No. 5,869,524 to Failli.

U.S. Pat. No. 5,869,660 to Adams et al.

U.S. Pat. No. 5,883,267 to Rossen et al.

U.S. Pat. No. 5,892,053 to Zhi et al.

U.S. Pat. No. 5,922,742 to Black et al.

U.S. Pat. No. 5,929,076 to Adams & Garigipati.

U.S. Pat. No. 5,932,598 to Talley et al.

U.S. Pat. No. 5,935,990 to Khanna et al.

U.S. Pat. No. 5,945,539 to Haruta et al.

U.S. Pat. No. 5,958,978 to Yamazaki et al.

U.S. Pat. No. 5,968,958 to Guay et al.

U.S. Pat. No. 5,972,950 to Nicolal & Teulon.

U.S. Pat. No. 5,973,191 to Marnett & Kalgutkar.

U.S. Pat. No. 5,981,576 to Belley et al.

U.S. Pat. No. 5,994,381 to Haruta et al.

U.S. Pat. No. 6,002,014 to Haruta et al.

U.S. Pat. No. 6,004,960 to Li et al.

U.S. Pat. No. 6,005,000 to Hopper et al.

U.S. Pat. No. 6,020,343 to Belley et al.

U.S. Pat. No. 6,020,347 to DeLaszlo & Hagmann.

Above-cited U.S. Pat. No. 6,034,256.

U.S. Pat. No. 6,040,319 to Corley et al.

U.S. Pat. No. 6,040,450 to Davies et al.

U.S. Pat. No. 6,046,208 to Adams et al.

U.S. Pat. No. 6,046,217 to Friesen et al.

U.S. Pat. No. 6,057,319 to Black et al.

U.S. Pat. No. 6,063,804 to De Nanteuil et al.

U.S. Pat. No. 6,063,807 to Chabrier de Lassauniere & Broquet.

U.S. Pat. No. 6,071,954 to LeBlanc et al.

U.S. Pat. No. 6,077,868 to Cook et al.

U.S. Pat. No. 6,077,869 to Sui & Wachter.

U.S. Pat. No. 6,083,969 to Ferro et al.

U.S. Pat. No. 6,096,753 to Spohr et al.

U.S. Pat. No. 6,133,292 to Wang et al.

International Patent Publication No. WO 94/15932.

International Patent Publication No. WO 96/19469.

International Patent Publication No. WO 96/26921.

International Patent Publication No. WO 96/31509.

International Patent Publication No. WO 96/36623.

International Patent Publication No. WO 96/38418.

International Patent Publication No. WO 97/03953.

International Patent Publication No. WO 97/10840.

International Patent Publication No. WO 97/13755.

International Patent Publication No. WO 97/13767.

International Patent Publication No. WO 97/25048.

International Patent Publication No. WO 97/30030.

International Patent Publication No. WO 97/34882.

International Patent Publication No. WO 97/46524.

International Patent Publication No. WO 98/04527.

International Patent Publication No. WO 98/06708.

International Patent Publication No. WO 98/07425.

International Patent Publication No. WO 98/17292.

International Patent Publication No. WO 98/21195.

International Patent Publication No. WO 98/22457.

International Patent Publication No. WO 98/32732.

International Patent Publication No. WO 98/41516.

International Patent Publication No. WO 98/43966.

International Patent Publication No. WO 98/45294.

International Patent Publication No. WO 98/47871.

International Patent Publication No. WO 99/01130.

International Patent Publication No. WO 99/01131.

International Patent Publication No. WO 99/01452.

International Patent Publication No. WO 99/01455.

International Patent Publication No. WO 99/10331.

International Patent Publication No. WO 99/10332.

International Patent Publication No. WO 99/11605.

International Patent Publication No. WO 99/12930.

International Patent Publication No. WO 99/14195.

International Patent Publication No. WO 99/14205.

International Patent Publication No. WO 99/15505.

International Patent Publication No. WO 99/23087.

International Patent Publication No. WO 99/24404.

International Patent Publication No. WO 99/25695.

International Patent Publication No. WO 99/35130.

International Patent Publication No. WO 99/61016.

International Patent Publication No. WO 99/61436.

International Patent Publication No. WO 99/62884.

International Patent Publication No. WO 99/64415.

International Patent Publication No. WO 00/01380.

International Patent Publication No. WO 00/08024.

International Patent Publication No. WO 00/10993.

International Patent Publication No. WO 00/13684.

International Patent Publication No. WO 00/18741.

International Patent Publication No. WO 00/18753.

International Patent Publication No. WO 00/23426.

Above-cited International Patent Publication No. WO 00/24719.

International Patent Publication No. WO 00/26216.

International Patent Publication No. WO 00/31072.

International Patent Publication No. WO 00/40087.

International Patent Publication No. WO 00/56348.

European Patent Application No. 0 799 823.

European Patent Application No. 0 846 689.

European Patent Application No. 0 863 134.

European Patent Application No. 0 985 666.

The compounds utilized in the methods of the present invention may be present in the form of free bases or pharmaceutically acceptable acid addition salts thereof. The term “pharmaceutically-acceptable salts” embraces salts commonly used to form alkali metal salts and to form addition salts of free acids or free bases. The nature of the salt may vary, provided that it is pharmaceutically-acceptable. Suitable pharmaceutically-acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, S-hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of Formula I or Formula II.

A. Dosage Information for Selective COX-2 Inhibitors

The dosage form and amount can be readily established by reference to known treatment or prophylactic regimens. The amount of therapeutically active compound that is administered and the dosage regimen for treating a disease condition with the compounds and/or compositions of this invention depends on a variety of factors, including the age, weight, sex and medical condition of the subject, the severity of the disease, the route and frequency of administration, and the particular compound employed, the location of the neoplasia, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual.

The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight and most preferably from about 1 to 20 mg/kg body weight, may be appropriate. The daily dose can be administered in one to four doses per day. A person skilled in the art will recognize that the particular dose amounts depend on the specific selective cyclooxygenase-2 inhibitor.

Where the drug is celecoxib, the composition typically comprises celecoxib in a therapeutically and/or prophylactically effective total amount of about 10 mg to about 1000 mg per dose unit. Where the drug is a selective COX-2 inhibitory drug other than celecoxib, the amount of the drug per dose unit is therapeutically equivalent to about 10 mg to about 1000 mg of celecoxib.

II. Vasomodulators Used in the Invention

There are large numbers of vasomodulator agents, vasoconstriction agents, vasodilation agents, bronchodilation agents, and bronchoconstriction agents available in commercial use, in clinical evaluation and in pre-clinical development, which could be selected for treatment of headache pain in combination with a selective cyclooxygenase-2 inhibitor. Some classes of vasomodulators that may be used in this invention are rennin-angiotensin system antagonists, nitrovasodilators, direct vasodilators, calcium channel blocking drugs, phosphodiesterase inhibitors, sympathomimetics, sympatholytics, and nitric oxide synthase inhibitors.

Examples of rennin-angiotensin system antagonists are Captopril (1-[(2S)-3-mercapto-2-methylpropionyl]-L-proline), Enalapril ((S)-1-[N-[1-(ethoxycarbonyl)-3-phenylpropyl]-L-alanyl]-L-proline, (Z)-2-butenedioate salt), Enalaprilal, Quinapril ((3S-(2(R*(R*)),3R*))-2-(2-((1-(ethoxycarbonyl)-3-phenylpropyl)amino)-1-oxopropyl)-1,2,3,4,-tetrahydro-3-isoquinolinecarboxylic acid, monohydrochloride), Lisinopril ((S)-1-[N²-(1-Carboxy-3-phenylpropyl)-L-lysyl]-L-proline dihydrate), Ramipril ((2S, 3aS, 6aS)-1-[(S)-N-[(S)-1-Carboxy-3-phenylpropyl]alanyl]octahydrocyclopenta[b]pyrrole-2-carboxylic acid, 1-ethyl ester), and Losartan (2-butyl-4-chloro-1[p-(o-1H-tetrazol-5-ylphenyl)benzyl]imidazole-5-methanol monopotassium salt). Examples of nitrovasodilators are nitroglycerin, isosobide dinitrate, and nitroprusside. Examples of direct vasodilators are hydralazine, Nicorandil, Minoxidil (2,4-diamino-6-piperidino-pyrimidine-3-oxide), and Diazoxide (3-methyl-7-chloro-1,2,4-benzothiadiazine-1,1-dioxide). Examples of calcium channel blocking drugs are Nifedipine (3,5-pyridinedicarboxylic acid, 1,4-dihydro-2,6-dimethyl-4-(2-nitrophenyl)-dimethyl ester), Amlodipine (3-ethyl-5-methyl-2-(2-aminoethoxymethyl)-4-(2-chlorophenyl)-1,4-dihydro-6-methyl-3,5-pyridinedicarboxylate benzenesulphonate), and Felodipine (+-ethyl methyl 4-(2,3-dichlorophenyl)-1,4-dihydro-2,6-dimethyl-3,5-pyridinedicarboxylate). Examples of phosphodiesterase inhibitors are Amrinone (5-amino(3,4′-bipyridin)-6(1H)-one), Milrinone (1,6-dihydro-2-methyl-6-oxo-[3,4′-bypyridine]-5-carbonitrile lactate), and Vesnarinone (3, 4-dihydro-6[4-(3,4-dimethoxybenzoyl)-1-piperazinyl]-2(1H)-quinolinone). Examples of a sympathomimetic are Dobutamine (1,2-benzenediol-4-[2-[3-(4-hydroxyphenyl)-1-methylpropyl]amino]ethyl-+-catecholamine), and Dopamine (4-(2-aminoethyl)pyrocatechol hydrochloride). Examples of sympatholytics are prazosin (1-(4-amino-6,7-dimethoxy-2-quinazonlinyl)-4-(2-furoyl)piperazine) (and other quinazoline derivatives), phentolamine (m-[N-(2-Imidazolin-2-ylmethyl)-p-toluidino]phenol monomethanesulfonate), Labetalol (2-hydroxy-5-[1-hydroxy-2-[(1-methyl-3-phenylpropyl)amino]ethyl]benzamide monohydrochloride), Carvedilol ((±)-1-Carbazol-4-yloxy)-3-[[2-(O-methoxyphenoxy)ethyl]aminol-2-propanol), and Bucindolol. The use of various vasomodulators in the present invention is not meant to be limited by this list of examples. A preferable vasomodulator for use in the present invention, to be used with a selective cyclooxygenase inhibitor, is a nitric oxide synthase inhibitor.

Additionally, the vasomodulator in the invention can be a xanthine compound. Preferably, the xanthine compound in this therapeutic combination is selected from the group consisting of caffeine, theobromine, theophylline, and xanthine. More preferably, the xanthine compound in this theraupeutic combination is selected from the group consisting of caffeine, theobromine, and theophylline. Still more preferably, the xanthine compound in this combination is selected from the group consisting of caffeine and theophylline, and most preferably, the xanthine compound in this therapeutic combination is caffeine.

A. Dosage Information for Vasomodulators

Typically, the preferable vasomodulator, caffeine, is administered in a daily dosage amount of about 1 to 500 mg. More preferably, the caffeine is administered in a daily dosage amount of about 10 to 400 mg. Still more preferably, caffeine is administered in a daily dosage amount of about 20 to 300 mg. Still more preferably, caffeine is administered in a daily dosage amount of about 30 to 200 mg. Yet more preferably, caffeine is administered in a daily dosage amount of about 40 to 150 mg. Most preferably, caffeine is administered in a daily dosage amount of about 55 to 100 mg.

III. Rapid-Onset Vehicles

The present invention can be delivered to a subject by two rapid-onset vehicles. First, the vehicle is a concentrated solution in the form of a discrete dose or an imbibable liquid. Second, the vehicle is a high energy phase composition of the selective COX-2 compound, illustratively, amorphous celecoxib, nanoparticulate celecoxib, dual-release celecoxib, and microparticulate valdecoxib.

Any combination of a one or more possible selections from each column in Table 3 may be selected to provide a therapeutic composition. For example, a selective cyclooxygenase-2 inhibitor and a vasomodulator may be delivered in any rapid onset vehicle in any form with any appropriate excipients. The non-limiting possible choices of selective cyclooxygenase-2 inhibitors, vasomodulator, rapid onset vehicle, form of drug substance, and excipients are listed in Table 3.

TABLE 3
Possible components of Drug Substance

| Selective | | | | |
|---|---|---|---|---|
| cyclooxy- | | Rapid | Forms of | |
| genase-2 | Vasomodul- | Onset | Drug | |
| Inhibitor | ator | Vehicle | Substance | Excipients |
| Any | vasomodul- | concen- | Discrete | free- |
| selective | ator | trated | dose | radical |
| COX-2 | | solution | | scavenging |
| described | | | | idant |
| in Section | | | | |
| I. above | | | | |
| | vasocon- | solution/ | Imbibable | fatty |
| | strictor | suspension | liquid | acid- |
| | | | | organic |
| | | | | amine pair |
| | vasodila- | amorphous | Tablet | crystalli- |
| | tor | component | | zation |
| | | | | inhibitor |
| | xanthine | nanopart- | Capsule | wetting |
| | | iculate | | agent |
| | | component | | |
| | caffeine | micropart- | Suspension | diluent |
| | | iculate | | |
| | | component | | |
| | | dual | Sterile | disinte- |
| | | release | aqueous | grant/eff- |
| | | | solution | ervescent |
| | | | | agent |
| | | | gels, | binding |
| | | | vreams, | agent/ad- |
| | | | oils | hesive |
| | | | Supposi- | lubricant |
| | | | tory | |
| | | | Lozenges | co-solvent |
| | | | | sweetener |
| | | | | preserva- |
| | | | | tive |
| | | | | dispersant |
| | | | | emulsify- |
| | | | | ing agent |
| | | | | buffering |
| | | | | agent |
| | | | | flavoring |
| | | | | agent |
| | | | | colorant |
| | | | | stabilizer |
| | | | | thickener |

A. Compositions in Solution

Compositions of the present invention are preferably in the form of a concentrated solution. A preferred embodiment of the invention is a composition comprising a therapeutically effective amount of a selective COX-2 inhibitor, for example celecoxib or valdecoxib, and a vasomodulator, substantially completely dissolved in a solvent liquid comprising at least one pharmaceutically acceptable polyethylene glycol. optionally, the concentrated solution can contain at least one pharmaceutically acceptable free radical-scavenging antioxidant. In this embodiment, substantially no part of the drug is present in solid particulate form. Compositions of this embodiment can be formulated either in an imbibable or discrete dosage form (e.g., encapsulated). Preferably, concentrated solutions of this embodiment have a drug concentration of about 10% to about 75%, more preferably about 20% to about 75%, by weight of the composition.

The invention is illustrated herein with particular reference to celecoxib, and it will be understood that any drug of low water solubility that comprises an aminosulfonyl functional group and/or is capable of reacting with a polyethylene glycol or a polyethylene glycol degradation product to form an addition compound can, if desired, be substituted in whole or in part for celecoxib in compositions herein described.

1. Solvent

Any pharmaceutically acceptable polyethylene glycol (PEG) can be used as a solvent in a composition of the invention. Preferably, the PEG has an average molecular weight of about 100 to about 10,000, and more preferably about 100 to about 1,000. Still more preferably, the PEG is of liquid grade. Non-limiting examples of PEGs that can be used in solvent liquids of this invention include PEG-200, PEG-350, PEG-400, PEG-540 and PEG-600. See for example Flick (1998): Industrial Solvents Handbook, 5th ed., Noyes Data Corporation, Westwood, N.J., p. 392. A presently preferred PEG has an average molecular weight of about 375 to about 450, as exemplified by PEG-400.

As pointed out hereinabove, PEGs such as PEG-400 have many desirable properties as solvents. In the case of celecoxib, for example, the drug can be dissolved or solubilized at a very high concentration in PEG-400, enabling formulation of a therapeutically effective dose in a very small volume of solvent liquid. This is especially important where the resulting solution is to be encapsulated, as capsules of a size convenient for swallowing can be prepared containing a therapeutically effective dose even of a drug such as celecoxib having a relatively high dose requirement for efficacy.

2. Free radical-scavenging Antioxidant

A composition of the present invention optionally comprises at least one pharmaceutically acceptable free radical-scavenging antioxidant. Non-limiting illustrative examples of suitable free radical-scavenging antioxidants include alpha-tocopherol (vitamin E), ascorbic acid (vitamin C) and salts thereof including sodium ascorbate and ascorbic acid palmitate, butylated hydroxyanisole (BHA), butylated hydroxytoluene (BHT), fumaric acid and salts thereof, hypophosphorous acid, malic acid, alkyl gallates, for example propyl gallate, octyl gallate and lauryl gallate, sodium sulfite, sodium bisulfite and sodium metabisulfite. Preferred free radical-scavenging antioxidants are alkyl gallates, vitamin E, BHA and BHT. More preferably the at least one free radical-scavenging antioxidant is propyl gallate.

One or more free radical-scavenging antioxidants are present in compositions of the invention in a total amount effective to substantially reduce formation of an addition compound, typically in a total amount of about 0.01% to about 5%, preferably about 0.01% to about 2.5%, and more preferably about 0.01% to about 1%, by weight of the composition.

3. Finely Self-emulsifiable Composition

A composition, particularly a solution composition, of the invention optionally comprises a pharmaceutically acceptable fatty acid and a pharmaceutically acceptable organic amine (also referred to herein as a “fatty acid/organic amine pair”) in absolute and relative amounts such that the composition is finely self-emulsifiable in simulated gastric fluid. “Simulated gastric fluid” and its abbreviation “SGF”, as the term is used herein, describes an aqueous solution of 0.01M hydrochloric acid and 0.15M sodium chloride, having a pH of about 2. Without being bound by theory, it is believed that a fatty acid/organic amine pair, when present in a composition of the invention, promotes formation of charged fine-emulsion droplets upon exposure of the composition to an aqueous medium such as SGF.

Whether a composition is “finely self-emulsifiable” in SGF as defined herein can illustratively be determined according to Test I.

Test I:

A. A 400 microliter aliquot of a test composition is placed into a screw-top, side-arm vessel containing 20 ml SGF (maintained at 37° C. throughout the test) to form a test liquid.

B. The test liquid is mildly agitated at 75 rpm for 2 minutes using an orbital shaker, to permit emulsification.

C. A 5-50 microliter aliquot of the test liquid is withdrawn through the side-arm using a pipette and is discharged from the pipette into a sampling vessel.

D. A pump (e.g., model RHOCKC-LF, Fluid Metering Inc., Syosset, N.Y.) is used to pull the sample from the sampling vessel through a combination scattering/obscuration sensor (e.g., LE400-0.5, Particle Sizing Systems, Santa Barbara, Calif.) at a rate of 1 ml/minute for a period of 1 minute.

E. Emulsion particles are counted individually by light scattering in the size (i.e., diameter) range from 0.5 to 1 micrometer and by light obscuration in the size range above 1 micrometer, using the vendor's software (e.g., Version 1.59).

F. A plot is prepared of number (i.e., unweighted) or volume (i.e., weighted) of emulsion particles versus particle diameter.

G. Integration of the plot, accounting for all dilutions, is performed to estimate total number or volume of emulsion particles present in the test liquid large enough to be detected by the sensor.

H. If Test I results in about 25% or more, by volume, of emulsion particles having a diameter of 1 micrometer or less, the test composition is deemed to be finely self-emulsifiable.

Preferred fatty acids have a saturated or unsaturated C₆₋₂₄ carbon chain. Non-limiting examples of suitable fatty acids include oleic acid, octanoic acid, caproic acid, caprylic acid, capric acid, eleostearic acid, lauric acid, myristic acid, palmitic acid, stearic acid, icosanoic acid, elaidic acid, linoleic acid, linolenic acid, eicosapentaenoic acid and docosahexaenoic acid. Oleic acid is an especially preferred fatty acid.

Preferred organic amines have a C₂₋₈ carbon chain with one or two amine groups. More preferably, organic amines can be selected from C₂₋₈ alkyl amines, alkylene diamines, alkanol amines, alkylalkanol amines, glycol ether amines and aryl amines. Non-limiting examples of suitable organic amines include monoethanolamine, diethanolamine, triethanolamine, dimethylaminoethanol, tromethamine, etc. Dimethylaminoethanol, monoethanolamine and tromethamine are especially preferred organic amines. Preferably, if present, a fatty acid/organic amine pair is selected (as to both type and amount of each component) such that when a composition of the invention is subjected to Test I, at least a substantial portion by volume of the emulsion particles counted, more preferably at least about 75%, still more preferably at least about 85%, and most preferably at least about 90%, of the emulsion particles counted, have a diameter of about 0.5 micrometer or less.

A preferred mole ratio of fatty acid to amine group(s) in the organic amine is about 5:1 to about 1:100, more preferably about 3:1 to about 1:50, and still more preferably about 2:1 to about 1:10, for example about 1:1. Preferably, if present, the fatty acid and organic amine are collectively present in an amount of about 1% to about 50%, more preferably about 2% to about 30%, and still more preferably about 5% to about 15%, by weight of the composition.

It is believed, without being bound by theory, that a finely self-emulsifiable solution composition of the invention, particularly one having a fatty acid/organic amine pair as described above, will provide the drug in a form that is especially rapidly absorbable in the gastrointestinal tract.

4. Crystallization Inhibitor

In a solution composition of the invention, the drug, even when finely emulsified, can, upon exposure to the aqueous environment of the gastrointestinal tract, precipitate and agglomerate in a solid, typically crystalline, particulate form. Such precipitation and/or crystallization can adversely impact any rapid-onset benefits obtained by administering a drug in dissolved form, because a drug that has reverted to a crystalline form must undergo the process of dissolution prior to absorption.

Therefore, preferred compositions further comprise a crystallization inhibitor comprising a cellulosic polymer wherein at least a portion of substitutable hydroxyl groups are individually substituted with methoxyl and/or hydroxypropoxyl groups. Preferably, the cellulosic polymer is water-soluble. More preferably the crystallization inhibitor is selected from hydroxypropylmethylcellulose (HPMC), methylcellulose and hydroxypropylcellulose. Still more preferably, the crystallization inhibitor is HPMC.

If included, the HPMC preferably has a viscosity, 2% in water, of about 100 to about 20,000 cP. HPMCs vary in the degree of substitution of available hydroxyl groups on the cellulosic backbone by methoxyl groups and by hydroxypropoxyl groups. With increasing hydroxypropoxyl substitution, the resulting HPMC becomes more hydrophilic in nature. It is preferred to use HPMC having about 15% to about 35%, more preferably about 19% to about 30%, and most preferably about 19% to about 24%, methoxyl substitution, and having about 3% to about 15%, more preferably about 4% to about 12%, and most preferably about 7% to about 12%, hydroxypropoxyl substitution.

Suitable HPMCs that are relatively hydrophilic in nature are illustratively available under the brand names Methocel™ of Dow Chemical Co. and Metolose™ of Shin-Etsu Chemical Co.

An illustrative presently preferred HPMC is one with substitution type 2208, denoting about 19% to about 24% methoxyl substitution and about 7% to about 12% hydroxypropoxyl substitution, and with a nominal viscosity, 2% in water, of about 4000 cP.

Surprisingly, it has been found that the crystallization inhibitor need not be a component of the solvent liquid. optionally, a crystallization inhibitor such as HPMC can be a component of a capsule wall wherein a solution composition of the invention is encapsulated. In one embodiment, substantially no HPMC or other crystallization inhibitor is present in the solvent liquid but the capsule wall comprises HPMC. The capsule wall can even consist predominantly of HPMC.

If present, the crystallization inhibitor is preferably present in a total amount sufficient to substantially inhibit drug crystallization and/or precipitation upon dilution of the composition in SGF. For practical purposes, whether an amount of crystallization inhibitor in a given test composition is sufficient to substantially inhibit drug crystallization and/or precipitation can be determined according to Test II, which can also be used to determine whether a particular polymer component is useful as a crystallization inhibitor in a particular composition of the invention.

Test II:

A. A volume of a test composition, either in unencapsulated or encapsulated form, having a polymer component is placed in a volume of SGF to form a mixture having a fixed ratio of about 1 g to about 2 g of the composition per 100 ml of SGF.

B. The mixture is maintained at a constant temperature of about 37° C. and is stirred using type II paddles (USP 24) at a rate of 75 rpm for a period of 4 hours.

C. At one or more time-points after at least about 15 minutes of stirring but before about 4 hours of stirring, an aliquot of the mixture is drawn and filtered, for example through a non-sterile Acrodisc” syringe filter with a 0.8 micrometer Versapor” membrane.

D. Filtrate is collected in a vessel.

E. Drug concentration in the filtrate is measured using high performance liquid chromatography (HPLC).

F. The test is repeated identically with a comparative composition that is substantially similar to the test composition except that it lacks the polymer component. Where the polymer component in the test composition is present in the solvent liquid, it is replaced in the comparative composition by polyethylene glycol solvent. Where the polymer component in the test composition is present in a capsule wall, it is replaced in the comparative composition with gelatin.

G. If the drug concentration in the filtrate resulting from the test composition is greater than that in the filtrate resulting from the comparative composition, the polymer component present in the test composition is deemed to substantially inhibit crystallization and/or precipitation of the drug in SGF.

A crystallization inhibitor such as HPMC, when present in the solvent liquid, is generally present in a total amount of about 1% to about 20%, preferably about 1% to about 15%, and most preferably about 1% to about 10%, by weight of the solvent liquid. Generally, the crystallization inhibitor, if present, and the drug are present in a ratio of about 1:100 to about 1:1, preferably about 1:50 to about 1:1 and more preferably about 1:25 to about 1:1, by weight.

5. Solution/Suspension Compositions

In one embodiment, the solvent liquid, depending on the particular components present therein, is suitable to maintain a first portion of drug in solution to provide a therapeutically effective rapid-onset dose while also maintaining a second portion of the drug undissolved but in suspension. The suspended portion typically provides less immediate release of the drug and so can extend the duration of therapeutic effect, although such extended duration is not a requirement of this embodiment of the invention.

Therefore, according to this embodiment a composition is provided comprising a therapeutically effective amount of a poorly water-soluble aminosulfonyl-comprising drug, in part dissolved and in part dispersed in a solvent liquid that comprises at least one pharmaceutically acceptable polyethylene glycol and at least one pharmaceutically acceptable free radical-scavenging antioxidant. In this embodiment, part of the drug is in solution and part is in suspension.

Preferably, the components of the solvent liquid are selected such that at least about 15% of the drug is in dissolved or solubilized form in the solvent liquid. One way of modifying a solvent liquid to increase the amount of the poorly water soluble aminosulfonyl-comprising drug in suspension as opposed to solution is to add water in an amount necessary to give the required reduction in solubility of the drug in the solvent liquid.

Depending on the relative importance of rapid onset and sustained action for the indication for which the drug is being administered, the relative proportions of dissolved and suspended drug can be varied significantly. For example, for acute pain indications, about 50% of the drug can be in solution and about 50% of the drug can be dispersed in particulate form. Alternatively, for indications demanding longer acting therapeutic effectiveness, illustratively about 20% of the drug can be in solution and about 80% of the drug can be dispersed in particulate form.

Through selection and combination of excipients, solution/suspension compositions can be provided exhibiting improved performance with respect to drug concentration, physical stability, efficacy, flavor, and overall patient compliance.

6. Forms for Oral Administration

a. Discrete Dosage Forms

Another embodiment of the present invention is a concentrated composition, either a solution or solution/suspension, wherein the composition is formulated as one or more discrete dose units, for example soft or hard capsules. Any suitable encapsulation material, for example gelatin or HPMC, can be used. As indicated hereinabove, HPMC can be an advantageous material for use in the capsule wall because it can act as a crystallization inhibitor upon exposure of the composition to gastrointestinal fluid.

If present, a cellulosic polymer having methoxyl and/or hydroxypropoxyl substitution as described hereinabove, preferably HPMC, is present in the capsule wall in a total amount of about 5% to substantially 100%, and preferably about 15% to substantially 100%, by weight of the wall. In addition to one or more such cellulosic polymers, a suitable capsule wall can comprise any additional component useful in the art such as gelatin, starch, carrageenan, sodium alginate, plasticizers, potassium chloride, coloring agents, etc. A suitable capsule herein may have a hard or soft wall.

Compositions of this embodiment are preferably formulated such that each discrete dosage unit contains about 0.3 ml to about 1.5 ml, more preferably about 0.3 ml to about 1 ml, for example about 0.8 ml or about 0.9 ml, of solution or solution/suspension.

Concentrated solutions or solutions/suspensions can be encapsulated by any method known in the art including the plate process, vacuum process, or the rotary die process. See, for example, Ansel et al. (1995) in Pharmaceutical Dosage Forms and Drug Delivery Systems, 6th ed., Williams & Wilkins, Baltimore, Md., pp. 176-182.

Capsules that comprise HPMC are known in the art and can be prepared, sealed and/or coated, by way of non-limiting illustration, according to processes disclosed in the Patents and publications listed below, each of which is individually incorporated herein by reference.

U.S. Pat. No. 4,250,997 to Bodenmann et al.

U.S. Pat. No. 5,264,223 to Yamamoto et al.

U.S. Pat. No. 5,756,123 to Yamamoto et al.

International Patent Publication No. WO 96/05812.

International Patent Publication No. WO 97/35537.

International Patent Publication No. WO 00/18377.

International Patent Publication No. WO 00/27367.

International Patent Publication No. WO 00/28976.

International Patent Publication No. WO 01/03676.

European Patent Application No. 0 211 079.

European Patent Application No. 0 919 228.

European Patent Application No. 1 029 539.

Non-limiting illustrative examples of suitable HPMC-comprising capsules include XGel™ capsules of Bioprogress and Qualicaps™ of Shionogi.

Preferably, one to about six, more preferably one to about four, and still more preferably one or two of such discrete dosage units per day provides a therapeutically effective dose of the drug.

b. Imbibable Liquids

Another embodiment of the present invention is a concentrated composition, either a concentrated solution or a concentrated solution/suspension, that can be directly imbibed or diluted with inert diluents and/or other carriers and imbibed; such compositions of the invention, whether diluted or not, are referred to for convenience herein as “imbibable compositions.” Imbibable compositions can be prepared by any suitable method of pharmacy that includes the steps of bringing into association the drug of low water solubility, illustratively celecoxib, and the solvent liquid. Where the drug is celecoxib, compositions of this embodiment preferably contain about 40 mg/ml to about 750 mg/ml, more preferably about 50 mg/ml to about 500 mg/ml, still more preferably about 50 mg/ml to about 350 mg/ml, and most preferably, about 100 mg/ml to about 300 mg/ml, for example about 200 mg/ml, of celecoxib.

In a further embodiment, solutions or solution/suspensions of the invention are provided that are required to be diluted to provide a dilution suitable for direct, imbibable administration. In this embodiment, solutions or solution/suspensions of the present invention are added, in a therapeutically effective dosage amount, to about 1 ml to about 20 ml of an inert liquid. Preferably solutions or solution/suspensions of the present invention are added to about 2 ml to about 15 ml, and more preferably to about 5 ml to about 10 ml, of inert liquid. The term “inert liquid” as used herein refers to pharmaceutically acceptable, preferably palatable liquid carriers. Such carriers are typically aqueous. Examples include water, fruit juices, carbonated beverages, etc.

B. High Energy Phase Compositions

A high energy phase composition of the present invention has a high energy compared to a perfect crystalline form of the invention. Thus, a high energy form of the invention can be a solution, suspension, solution/suspension, amorphous solid, nanoparticulate solid, or any solid wherein a substantial portion is non-crystalline.

Low energy, hydrophobic crystalline solids, due to their highly organized, lattice-like structures, typically require a significant amount of energy for dissolution. The energy required for a drug molecule to escape from a crystal, for example, is greater than is required for the same drug molecule to escape from a non-crystalline, amorphous form or from a higher energy crystalline polymorph. Therefore, a drug in a high energy phase can be more readily absorbed from the gastrointestinal tract into the blood stream than the same drug in a low energy crystalline state. Importantly, however, over time and upon contact with aqueous fluid, for example SGF, drugs in a high energy phase tend to revert to a steady state of low energy, for example to a stable, low energy crystalline state.

Therefore, another embodiment of the invention provides an orally deliverable pharmaceutical composition comprising a cyclooxygenase-2 inhibitor and a vasomodulator in a high energy phase together with one or more pharmaceutically acceptable excipients, encapsulated within a capsule wall that comprises a cellulosic polymer. The cellulosic polymer having at least a portion of substitutable hydroxyl groups substituted by methoxyl and/or hydroxypropoxyl groups, in an amount effective to substantially inhibit crystallization and/or precipitation of the drug in simulated gastric fluid.

The present combination of a selective COX-2 inhibitor and a vasomodulator may be formulated to provide a wide range of concentration profiles. The following paragraphs detail these formulations. In one embodiment of the invention, amorphous celecoxib is formulated with a vasomodulator to provide a composition with the desired pharmokinetic profile. In another embodiment, the blood plasma concentration of celecoxib reaches a concentration of about 250 ng/mL not later than about 30 minutes after oral administration. Another embodiment provides a dual release formulation consisting of nanoparticles for immediate release and microparticles for controlled release. An additional embodiment provides a rapid onset formulation composed of nanoparticles. Another embodiment increases bioavailability as determined by the threshold time to pain relief, the time to maximum concentration, and the maximum concentration profile of the composition.

1. Amorphous Celecoxib

The cyclooxygenase-2 inhibitor of the invention can be a novel amorphous form of celecoxib. The term “amorphous”, as used herein, refers to solid-state particles lacking a regular crystalline structure. Without being bound by theory, it is believed that amorphous celecoxib particles require less energy for dissolution than crystalline celecoxib particles of similar dimensions, and that this reduced dissolution energy requirement contributes, at least in part, to increased dissolution rate and/or decreased therapeutic onset time exhibited by amorphous celecoxib and compositions thereof.

The invention provides a celecoxib and vasomodulator drug substance that comprises amorphous celecoxib. At least a detectable amount of amorphous celecoxib is present. Preferably, about 10% to about 100%, more preferably about 25% to about 100%, still more preferably about 60% to about 100%, and even more preferably about 80% to about 100%, by weight of the celecoxib in a celecoxib drug substance of the invention is amorphous. In a particular embodiment, substantially all of the celecoxib is amorphous, i.e., the celecoxib drug substance is substantially phase pure amorphous celecoxib.

A preferred celecoxib-vasomodulator drug substance is an entirely solid-state substance wherein the fraction, if any, of the celecoxib that is not amorphous, is crystalline. This crystalline fraction is preferably small, for example, less than about 50%, more preferably less than about 25%, and still more preferably less than about 10%, by weight of the total celecoxib present.

In one embodiment, the amount of amorphous celecoxib compared with crystalline celecoxib is sufficient to provide increased dissolution rate as measured in a standard in vitro dissolution assay and/or improved bioavailability. For example, it provides a shorter time to reach a threshold therapeutic concentration in blood plasma, a greater Cₘₐₓ and/or a shorter Tₘₐₓ as measured in a standard in vivo pharmacokinetic study.

Amorphous celecoxib in a celecoxib-vasomodulator drug substance of the invention can be prepared by any suitable process, not limited to processes described herein.

One illustrative process comprises (a) a step of melting solid-state celecoxib, e.g., crystalline celecoxib; and (b) a step of rapidly cooling the resulting melted celecoxib to form a drug substance wherein the celecoxib is present, in at least a detectable amount, in amorphous form. This process optionally further comprises (c) a step of grinding the drug substance resulting from step (b) to form a drug powder.

Melting step (a) can be performed by any technique known in the art, for example, by heating the celecoxib in an oven at about 150° C. to about 180° C. Cooling step (b) is typically a quench cooling step that can be performed by any suitable method, for example by immersing a container holding the melted celecoxib in liquid nitrogen. The optional grinding step (c) can be performed by any suitable method, for example by grinding in a mortar and pestle or by grinding in a mill, for example a media mill.

Preferably, the drug substance or drug powder is subjected to further processing, typically with one or more excipients, to prepare a pharmaceutical composition, for example an oral dosage form, as described hereinbelow.

In a presently preferred embodiment of the invention there is provided a celecoxib-crystallization inhibitor composite combined with a vasomodulator comprising particles of amorphous celecoxib or a drug substance having at least a detectable amount of amorphous celecoxib, in intimate association with one or more crystallization inhibitors.

A celecoxib-crystallization inhibitor composite of this embodiment preferably comprises about 1% to about 95%, preferably about 10% to about 90%, more preferably about 25% to about 85%, and still more preferably about 30% to about 80%, by weight, of celecoxib. As indicated above, celecoxib in such a composite exists, at least in a detectable amount, in amorphous form. Preferably, about 10% to about 100%, more preferably about 50% to about 100%, and still more preferably about 75% to about 100%, by weight of the total celecoxib in the composite is amorphous celecoxib.

In composites of this embodiment, a fraction of the celecoxib can be present as microcrystalline or nanocrystalline celecoxib, though this fraction is preferably small, for example less than about 50%, more preferably less than about 25%, and still more preferably less than about 10%, by weight of the total celecoxib in the composite.

Crystallization inhibitors include any material, which substantially reduces conversion of amorphous celecoxib to crystalline celecoxib, for example, polymers, carbohydrates, lipids, etc. It will be understood that both selection of crystallization inhibitor(s) and the amount of crystallization inhibitor(s) used in a composite of the invention influences stability of amorphous celecoxib therein.

Crystallization inhibitors are preferably polymers, more preferably polymers of low solubility in water. Still more preferably, such polymers are substantially non-crosslinked.

Non-limiting examples of suitable polymers that can be used as crystallization inhibitors include, either alone or in combination, polyvinylpyrrolidone (PVP or povidone, e.g., Kollidon™ CLM of BASF), hydroxypropylmethylcellulose (HPMC, e.g., Methocel™ E5 Premium), HPMC phthalate, ethylcellulose, hydroxyethylcellulose, sodium carboxymethylcellulose (carmellose sodium), calcium carboxymethylcellulose, dextran, acacia, starches such as sodium starch glycolate (SSG, e.g., Explotab™ R of Mendell), -cyclodextrin (e.g., Kleptose™ 4PC of Roquette), block copolymers of ethylene oxide and propylene oxide (e.g., Pluronic™ F-68 and F-108), polyvinyl alcohol and polyethylene glycol (PEG). Povidone and HPMC are preferred polymers for use as crystallization inhibitors and form celecoxib-polymer composites of the invention.

HPMCs vary in the chain length of their cellulosic backbone and consequently in their viscosity as measured for example at a 2% by weight concentration in water. HPMC used in celecoxib-polymer composites of the invention should have a viscosity, 2% in water, of about 100 to about 100,000 cP, preferably about 1000 to about 15,000 cP, for example about 4000 cP. Molecular weight of HPMC used in celecoxib-polymer composites of the invention is preferably greater than about 10,000 but preferably not greater than about 1,500,000, more preferably not greater than about 1,000,000, still more preferably not greater than about 500,000, and even more preferably not greater than about 150,000.

HPMCs also vary in the relative degree of substitution of available hydroxyl groups on the cellulosic backbone by methoxy and hydroxypropoxy groups. With increasing hydroxypropoxy substitution, the resulting HPMC becomes more hydrophilic in nature. It is preferred in celecoxib-HPMC composites of the present invention to use HPMC having about 15% to about 35%, preferably about 19% to about 32%, and more preferably about 22% to about 30%, methoxy substitution, and having about 3% to about 15%, preferably about 4% to about 12%, and more preferably about 7% to about 12%, hydroxypropoxy substitution.

HPMCs which can be used in the present invention are illustratively available under the brand names Methocel™ of Dow Chemical Co. and Metolose™ of Shin-Etsu Chemical Co. Examples of particularly suitable HPMCs having medium viscosity include Methocel™ E4M and Methocel™ K4M, both of which have a viscosity, 2% in water, of about 4000 cP. Examples of HPMCs having higher viscosity include Methocel™ E10M, Methocel™ K15M and Methocel™ K100M, which have viscosities, 2% in water, of 10,000 cP, 15,000 cP and 100,000 cP respectively.

Preferred povidones used in celecoxib-polymer composites of the invention have a molecular weight of about 2,500 to about 3,000,000, preferably about 8,000 to about 1,000,000, and more preferably about 10,000 to about 400,000, for example, about 50,000. Preferably, povidone used in celecoxib-polymer composites have a dynamic viscosity, 10% in water at 20° C., of about 1.3 to about 700, preferably about 1.5 to about 300, and more preferably about 3.5 to about 8.5 mPa·s.

In celecoxib-crystallization inhibitor composites, when maintained in an open dish at ambient temperature for a period of 7 days, the amount of crystallization inhibitor is preferably sufficient to limit the transformation of amorphous celecoxib to crystalline celecoxib to no greater than about 50%, preferably no greater than about 25%, and more preferably no greater than about 10%, by weight of all celecoxib in the composite.

Typically, depending on the particular polymer(s) used, one or more polymers are present in a contemplated celecoxib-polymer composite in a total amount of about 10% to about 80%, preferably about 15% to about 75%, and more preferably about 25% to about 65%, by weight. Preferably, the weight ratio of celecoxib to polymer is about 1:1000 to about 10:1, more preferably about 1:10 to about 5:1, and still more preferably about 1:2 to about 2.5:1.

A celecoxib-crystallization inhibitor composite of the invention can be prepared by any suitable process, not limited to processes described herein.

One illustrative process comprises (a) a step of dissolving celecoxib and one or more crystallization inhibitors in a solvent liquid to form a solution; and (b) a step of drying the solution to form a celecoxib-crystallization inhibitor composite wherein the celecoxib and the crystallization inhibitor are in intimate association and wherein at least a detectable fraction of the celecoxib is in amorphous form. Optionally, this process can further comprise a step (c) of grinding the celecoxib-crystallization inhibitor composite to form a celecoxib-crystallization inhibitor composite powder.

Suitable solvent liquids which can be used to prepare a celecoxib-crystallization inhibitor composite, for example a celecoxib-polymer composite, can comprise any pharmaceutically acceptable solvent in which celecoxib can be dissolved. Heat and stirring can be used to facilitate drug dissolution in the solvent liquid. The solvent liquid can also comprise a non-solvent fraction, for example, water. Non-limiting examples of suitable solvents that may be used in solvent liquids of the invention include, for example, water-alcohol mixtures, methanol, ethanol, isopropanol, higher alcohols, propylene glycol, ethyl caprylate, propylene glycol laurate, PEG, diethyl glycol monoethyl ether (DGME), tetraethylene glycol dimethyl ether, triethylene glycol monoethyl ether, polysorbate 80, etc. Ethanol and isopropanol are preferred solvents.

Use of isopropanol as a solvent permits a relatively high loading of celecoxib and polymer in the solution to be dried. Accordingly, isopropanol is presently an especially preferred solvent.

The drying step (b) can be performed by any suitable means, for example, by evaporation, lyophilization, conventional heating (e.g., in an oven), spray drying, etc. Spray drying is a preferred method of drying. Any suitable spray drying method known in the art can be employed. The optional grinding step (c) can be performed by any suitable method.

2. Celecoxib Compositions for Fast Pain Relief

The present combination of selective cyclooxygenase-2 inhibitor and a vasomodulator provides a method of rapidly relieving pain in a mammalian subject, the method comprising orally administering to the subject an effective pain-relieving amount of a composition comprising celecoxib and a vasomodulator formulated in such a way as to provide, when tested in fasting humans in accordance with standard pharmacokinetic practice, a blood plasma concentration profile of celecoxib in which a concentration of about 250 ng/ml and a therapeutically effective amount of vasomodulator is attained not later than about 30 minutes after oral administration. Any formulation that provides the desired pharmacokinetic profile is included in this invention.

Celecoxib used in the method of the invention can be prepared by a process known per se, for example by processes described in U.S. Pat. No. 5,466,863 to Talley et al. or in U.S. Pat. No. 5,892,053 to Zhi & Newaz, both incorporated herein by reference.

A key to the present invention is selecting a formulation that provides a pharmacokinetic profile wherein a threshold blood plasma concentration of celecoxib of about 250 ng/ml is attained not later than about 30 minutes after oral administration. In preferred methods, a formulation is selected providing a higher concentration than about 250 ng/ml within about 30 minutes. For example, a formulation can be expected to be particularly effective for relief of pain if a blood plasma concentration of at least about 300 ng/ml, more preferably at least about 400 ng/ml and most preferably at least about 500 ng/ml, within about 30 minutes following oral administration of the formulation. There is no critical upper limit of blood plasma concentration so long as the dosage amounts set out above are not significantly exceeded; however it is likely that no significant incremental benefit will be obtained from blood plasma concentrations of celecoxib greatly in excess of about 500 ng/ml, for example in excess of about 1000 ng/ml, within the first 30 minutes.

Preferably, a threshold blood plasma concentration of celecoxib of about 250 ng/ml is attained not later than about 15 minutes after oral administration of the formulation.

In a particularly preferred embodiment the formulation provides a blood plasma concentration of celecoxib that attains about 300 ng/ml not later than about 30 minutes, most preferably not later than about 15 minutes, after oral administration.

In another particularly preferred embodiment the formulation exhibits a Tₘₐₓ not greater than about 1.25 hours, most preferably not greater than about 1 hour.

In yet another particularly preferred embodiment the formulation exhibits, in comparative pharmacokinetic testing versus a standard commercial formulation of celecoxib, such as Celebrex® 200 mg capsules of Pharmacia Corporation, a Tₘₐₓ not greater than about 50%, even more preferably not greater than about 33%, and most preferably not greater than about 25%, of the Tₘₐₓ exhibited by said standard commercial formulation.

Any standard pharmacokinetic protocol can be used to determine blood plasma concentration profile in humans following oral administration of a celecoxib formulation, and thereby establish whether that formulation meets the pharmacokinetic criteria set out herein.

Illustratively, a randomized single-dose crossover study can be performed using a group of healthy adult human subjects. The number of subjects is sufficient to provide adequate control of variation in a statistical analysis, and is typically about 10 or greater, although for certain purposes a smaller group can suffice. Each subject receives, by oral administration at time zero, a single dose (e.g., 200 mg) of a test formulation of celecoxib, normally at around 8 am following an overnight fast. The subject continues to fast and remains in an upright position for about 4 hours after administration of the celecoxib formulation. Blood samples are collected from each subject before administration (e.g., 15 minutes prior to administration) and at several intervals after administration. For the present purpose it is preferred to take several samples within the first hour, and to sample less frequently thereafter. Illustratively, blood samples can be collected 15, 30, 45, 60 and 90 minutes after administration, then every hour from 2 to 10 hours after administration. Optionally additional blood samples can be taken later, for example 12 and 24 hours after administration. If the same subjects are to be used for study of a second test formulation, a period of at least 7 days is allowed to elapse before administration of the second formulation. Plasma is separated from the blood samples by centrifugation and the separated plasma is analyzed for celecoxib by a validated high performance liquid chromatography (HPLC) procedure with a lower limit of detection of 10 ng/ml.

Any formulation giving the desired pharmacokinetic profile is suitable for administration according to the present method. One exemplary type of formulation giving such a profile has celecoxib ultra-finely dispersed in a liquid medium. If the liquid medium is one in which celecoxib is of very low solubility, for example an aqueous medium such as water or fruit juice, the celecoxib is present as suspended particles. The smaller the particles, the higher is the probability that the formulation will exhibit the presently desired pharmacokinetic profile. The ultimate in particle size reduction is represented by a true solution of celecoxib in a pharmaceutically acceptable solvent such as polyethylene glycol (PEG), e.g., PEG having an average molecular weight of about 400 (PEG-400), or a glycol ether, e.g., diethylene glycol monoethyl ether (DGME).

In a formulation having celecoxib in solid particulate form, it will generally be found necessary for practice of the present invention to provide celecoxib in a particle size range wherein D₉₀ is less than about 10 μm, for example about 10 nm to about 10 μm. Preferably, D₉₀ is less than about 2 μm. More preferably, the celecoxib is nanoparticulate, i.e., having D₉₀ less than about 1 μm.

In nanoparticulate celecoxib formulations, average particle size is preferably about 100 nm to about 800 nm, more preferably about 150 nm to about 600 nm, and most preferably about 200 nm to about 400 nm. Pharmaceutical compositions comprising such nanoparticulate celecoxib formulations represent a further embodiment of the present invention. Methods of preparing nanoparticulate celecoxib can be found hereinbelow.

a. Dose

A suitable dose of celecoxib, administered according to the method of the invention, is typically in the range of about 1 to about 6 mg/kg body weight, preferably about 1.3 to about 5.3 mg/kg body weight and more preferably about 2 to about 3.5 mg/kg body weight, for example about 2.7 mg/kg body weight. Depending on the body weight of the subject, a suitable dosage amount of celecoxib is typically about 50 to about 400 mg, preferably about 100 to about 300 mg. Surprisingly good results can be obtained with dosage amounts less than 300 mg, such as about 100 to about 275 mg, or about 150 to about 250 mg, for example about 200 mg.

The doses set out above relate to a single administration, and can be repeated as needed. Generally no more than about 4 doses per day will be needed, and in most cases 1 or 2 doses per day will be found sufficient.

b. Formulation

If the resulting suspension is allowed to stand, the celecoxib particles tend to agglomerate and/or increase in size by crystal growth. These processes can occur relatively quickly. It is therefore important that the suspension be administered as soon as possible after preparation, preferably not more than about 15 minutes and most preferably not more than about 5 minutes after preparation.

Finely divided particulate or nanoparticulate celecoxib is not necessarily administered in suspension. It can be administered as a solid dosage form such as a capsule or tablet, provided disintegration of the solid dosage form to release celecoxib into the gastrointestinal fluid occurs rapidly enough to generate the presently desired pharmacokinetic profile. Similarly, a solution of celecoxib can be administered in a capsule, such as a soft gelatin capsule, provided the capsule wall dissolves or disintegrates rapidly enough in gastrointestinal fluid to enable the celecoxib thus released to be absorbed into the bloodstream and generate the presently desired pharmacokinetic profile.

Celecoxib is highly hydrophobic; inclusion in the formulation of a wetting agent can provide wetting of celecoxib particles and can improve absorption. This can also help provide a pharmacokinetic profile consistent with the present invention, even where particle size is not ideal. Any suitable wetting agent can be used; presently preferred examples include polysorbate 80 and sodium lauryl sulfate.

3. Dual-release Celecoxib Compositions

The combination of a selective cyclooxygenase-2 inhibitor and a vasomodulator can be formulated to provide a greater maximum blood serum celecoxib concentration (Cₘₐₓ) and/or a shorter time following the administration to reach that maximum (Tₘₐₓ) and a longer terminal half-life of blood serum celecoxib cocentration (T_{½}). The formulation accomplishes this through a first fraction of celecoxib in solution having a D₉₀ particle size less than about 1 μm and a second fraction of celecoxib in solid form having a D₉₀ particle size greater than about 25 μm and/or in controlled-release, slow-release, programmed-release, timed-release, pulse-release, sustained-release, or extended-release particles. Also, a method of treating a medical condition with the above formulation is detailed.

In one embodiment, the first fraction of celecoxib in a composition of the invention, which is the fraction providing immediate release, is in the form of particles having a D₉₀ particle size less than about 1lm. Typically in this embodiment substantially all the particles are nanoparticles. In such particles, celecoxib and a vasomodulator can be present alone or in intimate mixture with one or more excipients.

The effects on pharmacokinetic properties of reducing particle size from the microparticle range (greater than 1 μm diameter) to the nanoparticle range is generally unpredictable for any particular drug or class of drugs. According to the present invention, celecoxib in nanoparticulate form exhibits higher Cₘₐₓ and/or shorter Tₘₐₓ than celecoxib in microparticulate form.

Considering only the nanoparticulate component of a composition of this embodiment of the invention, average particle size is preferably about 100 to about 800 nm, more preferably about 150 to about 600 nm, and most preferably about 200 to about 400 nm. Celecoxib can be in crystalline or amorphous form in the nanoparticles.

In one embodiment, celecoxib nanoparticles have a surface modifying agent adsorbed on the surface thereof. In another embodiment, celecoxib nanoparticles are contained in a matrix formed by a polymer. Preferably excipients are present and most preferably include a water soluble diluent or wetting agent. Such a water soluble diluent or wetting agent assists in the dispersion and dissolution of the celecoxib when a nanoparticulate composition is ingested. Preferably both a water soluble diluent and a wetting agent are present.

In another embodiment, the first fraction of celecoxib in a composition of the invention, which is the fraction providing immediate release, is in solution in a pharmaceutically acceptable solvent. Polyethylene glycol, for example PEG-400, has been found to be a suitable solvent, either alone or in mixture with water. Illustratively, a mixture of 2 parts PEG-400 to 1 part water has been found to be a useful solvent base for an orally deliverable celecoxib solution. According to the present invention, orally administered celecoxib in dissolved form exhibits higher Cₘₐₓ and/or shorter Tₘₐₓ than celecoxib in any other orally administered form so far evaluated.

When administered orally to a fasting adult human, a 100 mg dose unit of a composition of the invention preferably exhibits a Tₘₐₓ of less than about 1.5 h, more preferably less than about 1 h and most preferably less than about 0.75 h, and a Cₘₐₓ of at least about 100 ng/ml, more preferably at least about 200 ng/ml. Typically a composition of the invention provides a blood serum concentration of celecoxib of at least about 50 ng/ml within 30 minutes of oral administration; preferred compositions achieve such a concentration in as little as 15 minutes. This early rise in blood serum concentration is believed to be associated with the rapid onset of therapeutic effect achieved by compositions of the present invention.

In addition to the first fraction of celecoxib, which as explained above is the immediate-release fraction, a composition of the invention further comprises a second fraction of celecoxib that is the controlled-release, slow-release, programmed-release, timed-release, pulse-release, sustained-release or extended-release fraction. In one embodiment, this fraction comprises celecoxib microparticles having a Dgo particle size greater than about 25 μm. Preferably the D₉₀ particle size of this fraction is about 25μm to about 200μm, more preferably about 25μm to about 100 μm, for example about 40 μm to about 75 μm.

In another embodiment, the second fraction of celecoxib is in the form of particles of any convenient size that are controlled-release, slow-release, programmed-release, timed-release, pulse-release, sustained-release or extended-release particles prepared by any process disclosed for drugs other than celecoxib in the above-cited documents, such process being adapted as necessary for the specific properties of celecoxib.

The particles comprising the second fraction of celecoxib can optionally be dispersed as a suspension in a liquid diluent. In one embodiment of the invention, the particles comprising the second fraction are in stable suspension in a matrix solution comprising the first fraction of celecoxib. This suspension can be presented as a bulk liquid or can be in a pre-measured dosage form such as soft capsules, optionally as softgels or gelcaps as described above.

When administered orally to a fasting adult human, a 100 mg dose unit of a composition of the invention preferably exhibits a T_{½} of at least about 9 h, more preferably at least about 12 h and most preferably at least about 15 h. The T_{½} is preferably such as to maintain a blood serum concentration of celecoxib of at least about 50 ng/ml, preferably at least about 100 ng/ml, for about 18 h, more preferably for about 24 h, following administration. This maintenance of blood serum concentration is believed to be associated with the long duration of therapeutic effect achieved by oral administration of a single dose of a composition of the present invention. In particular, it is believed that this maintenance of blood serum concentration is what enables a once-a-day administration regimen for preferred compositions of the invention.

a. Dose

One embodiment of the invention is a pharmaceutical composition comprising one or more orally deliverable dose units, each comprising a first fraction of celecoxib in immediate-release form in an amount of about 10 mg to about 400 mg, and a second fraction of celecoxib in controlled-release, slow-release, programmed-release, timed-release, pulse-release, sustained-release or extended-release form in an amount of about 10 mg to about 400 mg, this composition providing, upon a single administration of 1 to about 4 dose units to a subject, (a) a Cₘₐₓ greater than about 100 ng/ml, (b) a Tₘₐₓ shorter than about 1.5 h and (c) a T_{½} longer than about 9 h.

A preferred composition provides, upon a single administration of 1 to about 4 dose units to a subject, (a) a Cₘₐₓ greater than about 200 ng/ml, (b) a Tₘₐₓ shorter than about 0.75 h, (c) a blood serum concentration of at least 50 ng/ml, preferably at least 100 ng/ml, within about 15 minutes after such administration, and (d) a T_{½} such that blood serum concentration remains above about 50 ng/ml, preferably above about 100 ng/ml, for at least 18 h, preferably at least 24 h, after such administration.

A preferred composition has pharmacokinetic properties sufficient to provide rapid onset of therapeutic effect within about 1 h, and a duration of therapeutic effect of at least about 24 h, after oral administration thereof to a subject having a cyclooxygenase-2 mediated disorder A particularly preferred composition has the first fraction of celecoxib in an immediate-release form and the second fraction of celecoxib in a pulse-release form that releases a pulse of celecoxib about 8 h to about 12 h after administration.

The weight ratio of the first to the second fraction of celecoxib in a composition of the invention is about 1:10 to about 10:1, preferably about 1:5 to about 5:1, for example about 1:1 or about 1:2.

In general, a composition of the invention is preferably administered at a dose suitable to provide an average blood serum concentration of celecoxib of at least about 100 ng/ml in a subject over a period of about 24 hours after administration.

b. Formulation

Preferably excipients are associated with or present in the primary microparticles and these excipients more preferably include a water soluble diluent or wetting agent. Most preferably both a water soluble diluent and a wetting agent are present.

4. Rapid-onset Cyclooxygenase-2 Inhibitor Compositions

The present combination of a cyclooxygenase-2 inhibitor and a vasomodulator can be formulated to provide a composition that exhibits pharmacokinetic properties leading to a greater maximum blood serum concentration (Cmax) and/or a shorter time following the administration to reach that maximum (Tmax). This pharmacokinetic profile is attained by reducing the particle size of the cyclooxygenase-2 particles so that a substantial portion are smaller than 1 μm in diameter, in the longest dimension of the particles. Without being bound by theory, it is believed that the composition has a short dissolution time due to the substantial portion of the particles having a particle size less than 1 μm.

Compositions of the present invention contain a selective cyclooxygenase-2 inhibitor, illustratively celecoxib, and a vasomodulator alone or in intimate mixture with one or more excipients, in nanoparticulate form.

As described hereinabove, nanoparticulate compositions of cyclooxygenase-2 inhibitors exhibit higher Cₘₐₓ and/or shorter Tₘₐₓ than microparticulate compositions. In one embodiment of the invention, therefore, the percentage by weight of the particles that are nanoparticles is sufficient to provide a substantially higher Cₘₐₓ and/or a substantially shorter Tₘₐₓ by comparison with a comparative composition wherein substantially all of the particles are larger than 1 μm. Preferably a composition of this embodiment has a sufficient percentage by weight of nanoparticles to provide a substantially shorter Tₘₐₓ, and more preferably a sufficient percentage by weight of nanoparticles to provide both a substantially higher Cₘₐₓ and a substantially shorter Tₘₐₓ, than the comparative composition.

When administered orally to a fasting adult human, a 100 mg dose unit preferably exhibits a Tₘₐₓ of less than about 90 minutes, more preferably less than about 60 minutes and most preferably less than about 45 minutes, and a Cₘₐₓ of at least about 100 ng/ml, more preferably at least about 200 ng/ml. Typically a composition of the invention provides a blood serum concentration of the selective cyclooxygenase-2 inhibitor of at least about 50 ng/ml within 30 minutes of oral administration; preferred compositions achieve such a concentration in as little as 15 minutes. This early rise in blood serum concentration is believed to be associated with the rapid onset of therapeutic effect achieved by compositions of the present invention.

In another embodiment of the invention, the selective cyclooxygenase-2 inhibitor, illustratively celecoxib, is present in solid particles having a D₉₀ particle size of about 0.01 to about 200 μm, wherein about 25% to 100% by weight of the particles are nanoparticles. Where the percentage by weight of nanoparticles is relatively low, for example about 25% to about 50%, preferably the D₉₀ particle size is about 0.01 to about 100 μm, more preferably about 0.01 to about 75 μm, still more preferably about 0.01 to about 40 μm, and even more preferably about 0.01 to about 25 μm. Particle size can vary continuously across the nanoparticulate and microparticulate range, or the composition can have a bimodal or multimodal particle size distribution, with one set of particles having a D₉₀ particle size less than 1 μm and another set of particles having a D₉₀ particle size substantially greater than lm. It is generally preferred that at least about 50% by weight, and especially preferred that at least about 75% by weight, of the particles are nanoparticles. In one embodiment substantially all of the particles are smaller than 1 μm, i.e., the percentage by weight of nanoparticles is 100% or close to 100%.

Considering only the nanoparticulate component of a composition of the invention, average particle size is preferably about 100 to about 800 nm, more preferably about 150 to about 600 nm, and most preferably about 200 to about 400 nm. The selective cyclooxygenase-2 inhibitor, illustratively celecoxib, can be in crystalline or amorphous form in the nanoparticles.

a. Dose

It will be understood that a therapeutically effective amount of a selective cyclooxygenase-2 inhibitor for a subject is dependent inter alia on the body weight of the subject. Where the cyclooxygenase-2 inhibitor is celecoxib, the preferred range of about 10 mg to about 1000 mg is likely to provide blood serum concentrations consistent with therapeutic effectiveness.

Typical dose units in a composition of the invention contain about 10, 20, 25, 37.5, 50, 75, 100, 125, 150, 175, 200, 250, 300, 350 or 400 mg of the cyclooxygenase-2 inhibitor, illustratively celecoxib. For an adult human, a therapeutically effective amount of celecoxib per dose unit in a composition of the present invention is typically about 50 mg to about 400 mg. Especially preferred amounts of celecoxib per dose unit are about 100 mg to about 200 mg, for example about 100 mg or about 200 mg.

5. Valdecoxib Compositions

The present combination of a cyclooxygenase-2 inhibitor and a vasomodulator can be formulated to provide a desired pharmokinetic profile. Accordingly, a combination of, illustratively, valdecoxib and a vasomodulator can be formulated to provide a time course of blood serum concentration of valdecoxib having at least one of the following:

a time to reach a threshold concentration for therapeutic effect not greater than about 0.5 h after administration;

a time to reach maximum concentration (Tmax) not greater than about 5 h after administration; and

a maximum concentration (Cmax) not less than about 100 ng/ml.

Thus, depending on the desired pharmokinetic profile, an acceptable composition can be formulated.

It is believed, without being bound by theory, that the strong clinical benefits afforded by a composition of the invention result from improved bioavailability of valdecoxib, in particular from surprisingly effective absorption of valdecoxib in the gastrointestinal tract when administered orally in such a composition. Such effective absorption can be verified by one of skill in the art by monitoring blood serum concentration of valdecoxib in a treated subject for a period of time following administration. It is desired to reach, in as short a time as possible, a threshold of valdecoxib concentration in the blood serum consistent with effective COX-2 inhibition.

As indicated above, in one embodiment a single dose, upon oral administration to a fasting subject, provides a time course of blood serum concentration of valdecoxib having at least one of the following:

a time to reach a threshold concentration for therapeutic effect (typically at least about 20 ng/ml) not greater than about 0.5 h after administration;

a time to reach maximum concentration (Tmax) not greater than about 5 h after administration; and

a maximum concentration (Cmax) not less than about 100 ng/ml.

In a preferred embodiment, the bioavailability of the composition is such that, when a 20 mg dose is administered orally to a fasting adult human subject:

a valdecoxib blood serum concentration of 20 ng/ml, more preferably of 50 ng/ml, is reached not more than about 0.5 h after administration;

Tₘₐₓ is not greater than about 3 h after administration; and

Cₘₐₓ is not less than about 100 ng/ml.

Compositions of the invention contain valdecoxib in particulate form. Primary valdecoxib particles, generated for example by milling or grinding, or by precipitation from solution, can agglomerate to form secondary aggregate particles. Particle size is believed to be an important parameter affecting clinical effectiveness of valdecoxib. Thus, in one embodiment, a composition has a distribution of valdecoxib particle sizes such that the D₉₀ particle size is less than about 75 μm.

In addition or alternatively, valdecoxib particles in a composition of the invention preferably have a weight average particle size of about 1 μm to about 10 μm, most preferably about 5 μm to about 7 μm.

Particle size reduction of the valdecoxib can lead to improved bioavailability when the drug is formulated as an orally deliverable composition in accordance with the invention. Accordingly, the D₉₀ particle size of the valdecoxib is preferably less than about 75 μm, even more preferably less than about 40 μm, and most preferably less than about 25 μm. In addition or alternatively, the valdecoxib preferably has a weight average particle size in the range of about 1 μm to about 10 μm, more preferably about 5 μm to about 7 μm. Any suitable milling, grinding or micronizing method can be used for particle size reduction.

a. Dose

A composition of the invention comprises particulate valdecoxib in a dosage amount of about 1 mg to about 100 mg. As described hereinabove for other cyclooxygenase-2 inhibitors, the amount of valdecoxib in a dose unit effective to provide blood serum concentrations meeting any of criteria (a) to (c) immediately above is dependent on the body weight of the treated subject. For an adult human, a suitable amount of valdecoxib per dose in a composition of the present invention to provide the indicated blood serum concentrations is typically about 5 mg to about 40 mg.

b. Formulations

Capsule and tablet compositions of the invention are immediate release compositions that release at least about 50%, more preferably at least about 60% and most preferably at least about 75% of the valdecoxib, as measured in vitro in a standard dissolution assay, within about 45 minutes.

Especially preferred capsule and tablet compositions of the invention release in vitro at least about 50% of the valdecoxib within about 15 minutes, and/or at least about 60% of the valdecoxib within about 30 minutes.

Preferred compositions of the invention comprise valdecoxib together with one or more excipients selected from diluents, disintegrants, binding agents, wetting agents and lubricants. In one preferred embodiment at least one of the excipients is a water-soluble diluent or wetting agent. Such a water-soluble diluent or wetting agent is believed to assist in dispersion and dissolution of the valdecoxib in the gastrointestinal tract. Preferably at least a water-soluble diluent is present. In another preferred embodiment at least one of the excipients is a disintegrant. In another preferred embodiment at least one of the excipients is a binding agent; as indicated above, it is particularly preferred that pregelatinized starch be present as a binding agent. In another preferred embodiment at least one of the excipients is a lubricant. It is esepcially preferred that the composition comprise, in addition to valdecoxib, each of a water-soluble diluent, a disintegrant, a binding agent and a lubricant.

C. Types of Pain for Treatment with Invention

In a therapeutic combination for the treatment, prevention, inhibition, or amelioration of pain consisting essentially of a selective cyclooxygenase-2 inhibitor compound a vasomodulator, the pain can be generalized pain or headache pain. The headache pain can be from migraine headache pain, cluster headache pain, chronic daily headache pain, substance-induced headache pain, tension or stress related headache pain, sinus headache pain, pain resulting from anesthesia, headache pain associated with increased intracranial pressure, headache pain associated with decreased intracranial pressure, headache pain resulting from giant cell arteritis, or headache pain resulting from lumbar puncture. A very important preference for this invention is pain which results from migraine pain. Another important preference in the present invention is pain resulting from a cluster headache. Another preferred source of pain for this invention is chronic headache pain. Still another preferred pain is substance-induced headache pain. Tension or stress related headache pain is another very important source of pain for the present invention. Pain resulting from anesthesia is another preference for the current invention. Pain resulting from changes in intracranial pressure is another very important source of pain for the present invention. An increase in intracranial pressure is a preferred source of pain for this invention. A decrease in intracranial pressure is another important source of headache pain for this invention. Preferably, the source of headache pain for the present invention is sinus headache pain. Headache pain from giant cell arteritis is another crucial source of headache pain for the present invention. Lumbar puncture can produce severe headache pain and is therefore another preferred source of pain for the embodiments of this invention.

The method of the invention can be used to relieve acute or chronic pain, but is particularly well-suited to acute pain indications such as post-surgical pain or post-traumatic pain.

Additionally, the therapeutic combination of a selective cyclooxygenase-2 inhibitor and a vasomodulator can be used to treat apnea and asthma.

D. Formulations

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, intraperitoneal, or rectal administration, the compound may be combined with a sterile aqueous solution that is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as sealed ampoules or vials.

Formulations suitable for parenteral administration conveniently comprise a sterile aqueous preparation of the active compound that is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

Formulations for topical use include known gels, creams, oils, and the like. For aerosol delivery, the compounds may be formulated with known aerosol exipients, such as saline, and administered using commercially available nebulizers. Formulation in a fatty acid source may be used to enhance biocompatibility. Aerosol delivery is an important method of delivery for nasal delivery.

As indicated above, the invention provides a pharmaceutical composition suitable for topical administration to an eye. The composition comprises a selective COX-2 inhibitory drug in a concentration effective for treatment and/or prophylaxis of a COX-2 mediated disorder in the eye, and one or more ophthalmically acceptable excipient ingredients that reduce rate of removal of the composition from the eye by lacrimation, such reduction in rate of removal including rendering the composition resistant to removal from the eye by lacrimation. By virtue at least in part of this reduced rate of removal by lacrimation, the composition has an effective residence time in the eye of about 2 to about 24 hours.

In one embodiment, the selective COX-2 inhibitory drug is of low water solubility, for example having a solubility of less than about 1 mg/ml.

Preferably the composition has an effective residence time in the eye of about 3 to about 24 hours, more preferably about 4 to about 24 hours and most preferably about 6 to about 24 hours.

A composition of the invention can illustratively take the form of a liquid wherein the drug is present in solution, in suspension or both.

A liquid composition herein includes a gel. Preferably the liquid composition is aqueous. Alternatively, the composition can take the form of an ointment.

As a further alternative, the composition can take the form of a solid article that can be inserted between the eye and eyelid or in the conjunctival sac, where it releases the drug as described, for example, in U.S. Pat. No. 3,863,633 and U.S. Pat. No. 3,868,445, both to Ryde & Ekstedt, incorporated herein by reference. Release is to the lacrimal fluid that bathes the surface of the cornea, or directly to the cornea itself, with which the solid article is generally in intimate contact. Solid articles suitable for implantation in the eye in such fashion are generally composed primarily of polymers and can be biodegradable or non-biodegradable. Biodegradable polymers that can be used in preparation of ocular implants carrying a selective COX-2 inhibitory drug in accordance with the present invention include without restriction aliphatic polyesters such as polymers and copolymers of poly(glycolide), poly(lactide), poly(caprolactone), poly(hydroxybutyrate) and poly(hydroxyvalerate), polyamino acids, polyorthoesters, polyanhydrides, aliphatic polycarbonates and polyether lactones. Illustrative of suitable non-biodegradable polymers are silicone elastomers.

In a presently preferred embodiment, the composition is an aqueous solution, suspension or solution/suspension, which can be presented in the form of eye drops. By means of a suitable dispenser, a desired dosage of the drug can be metered by administration of a known number of drops into the eye. For example, for a drop volume of 25 microliter, administration of 1-6 drops will deliver 25-150 microliter of the composition. Aqueous compositions of the invention preferably contain from about 0.01% to about 50%, more preferably about 0.1% to about 20%, still more preferably about 0.2% to about 10%, and most preferably about 0.5% to about 5%, weight/volume of the selective COX-2 inhibitory drug. In one embodiment, a composition of the invention contains a concentration of the selective COX-2 inhibitory drug that is therapeutically or prophylactically equivalent to a celecoxib weight/volume concentration of about 0.1% to about 50%, preferably about 0.5% to about 20%, and most preferably about 1% to about 10%. In another embodiment, a composition of the invention has relatively high loading of the drug and is suitable for a relatively long residence time in a treated eye. In this embodiment the weight/volume concentration of the drug in the composition is about 1.3% to about 50%, preferably about 1.5% to about 30%, and most preferably about 2% to about 20%, for example about 2% to about 10%.

Preferably no more than 3 drops, more preferably no more than 2 drops, and most preferably no more than 1 drop, each of about 15 to about 40 microliters, preferably about 20 to about 30 microliters, for example about 25 microliters, should contain the desired dose of the drug for administration to an eye. Administration of a larger volume to the eye risks loss of a significant portion of the applied composition by lacrimation.

Aqueous compositions of the invention have ophthalmically compatible pH and osmolality.

In an aqueous suspension or solution/suspension composition of a preferred embodiment of the invention, the selective COX-2 inhibitory drug is present predominantly in the form of nanoparticles, i.e., solid particles smaller than about 1 micrometer in their longest dimension. A benefit of this embodiment is more rapid release of the drug, and therefore more complete release during the residence time of the composition in a treated eye, than occurs with larger particle size. Another benefit is reduced potential for eye irritation by comparison with larger particle size. Reduced eye irritation in turn leads to a reduced tendency for loss of the composition from the treated eye by lacrimation, which is stimulated by such irritation.

In a related embodiment the drug preferably has a D₉₀ particle size of about 0.01 to about 200 micrometer, wherein about 25% to 100% by weight of the particles are nanoparticles.

An aqueous suspension composition of the invention can comprise a first portion of the drug in nanoparticulate form, to promote relatively rapid release, and a second portion of the drug having a D₉₀ particle size of about 10 micrometer or greater, that can provide a depot or reservoir of the drug in the treated eye for release over a period of time, for example about 2 to about 24 hours, more typically about 2 to about 12 hours, to promote sustained therapeutic effect and permit a reduced frequency of administration.

In a particular embodiment the composition is an in situ gellable aqueous solution, suspension or solution/suspension having excipients substantially as disclosed in above-cited U.S. Pat. No. 5,192,535, comprising about 0.1% to about 6.5%, preferably about 0.5% to about 4.5%, by weight, based on the total weight of the composition, of one or more cross-linked carboxyl-containing polymers. Such an aqueous suspension is preferably sterile and has an osmolality of about 10 to about 400 mOsM, preferably about 100 to about 250 mOsM, a pH of about 3 to about 6.5, preferably about 4 to about 6, and an initial viscosity, when administered to the eye, of about 1000 to about 30,000 cPs, as measured at 250C using a Brookfield Digital LVT viscometer with #25 spindle and 13R small sample adapter at 12 rpm. More typically the initial viscosity is about 5000 to about 20,000 cPs. The polymer component has an average particle size not greater than about 50 micrometers, preferably not greater than about 30 micrometers, more preferably not greater than about 20 micrometers, and most preferably about 1 micrometer to about 5 micrometer, in equivalent spherical diameter, and is lightly cross-linked to a degree such that, upon contact with tear fluid in the eye, which has a typical pH of about 7.2 to about 7.4, the viscosity of the suspension rapidly increases, to form a gel.

For rectal administration, the active ingredient may be formulated into suppositories using bases that are solid at room temperature and melt or dissolve at body temperature. Commonly used bases include coca butter, glycerinated gelatin, hydrogenated vegetable oil, polyethylene glycols of various molecular weights, and fatty esters of polyethylene stearate.

In another preferred embodiment of the present invention, a therapeutic combination of a selective cyclooxygenase-2 inhibitor and a vasomodulator are administered combined in a single dosage form. Preferably, the vasomodulator compound is caffeine. Preferably the selective cyclooxygenase-2 inhibitor and the second agent, whether it is a vasomodulator, a vasoconstrictor, a vasodilator, or a xanthine compound, are administered combined in a single dosage form. Preferably, a therapeutic combination administered combined in a single dosage form is a single tablet, pill or capsule of said single dosage form comprising a selective cyclooxygenase-2 inhibitor in an amount of from about 0.1 mg to about 2000 mg, and caffeine in an amount of about 1 to 500 mg. More preferably, a single tablet, pill or capsule of said single dosage form comprises a selective cyclooxygenase-2 inhibitor is in an amount of from about 0.5 mg to about 500 mg, and caffeine in an amount of about 10 to 400 mg. Still more preferably, the dosage form comprises a selective cyclooxygenase-2 inhibitor in an amount of from about 1 mg to about 200 mg, and caffeine in an amount of about 20 to 300 mg. Still more preferably, the dosage form comprises a selective cyclooxygenase-2 inhibitor in an amount of from about 1 mg to about 200 mg, and caffeine in an amount of about 30 to 200 mg. Yet more preferably, the dosage form comprises a selective cyclooxygenase-2 inhibitor in an amount of from about 1 mg to about 200 mg, and caffeine in an amount of about 40 to 150 mg. More preferably, the dosage form comprises a selective cyclooxygenase-2 inhibitor in an amount of from about 1 mg to about 200 mg, and caffeine in an amount of about 55 to 100 mg.

In another preferred embodiment of the present invention, a therapeutic combination, wherein the first agent is a selective cyclooxygenase-2 inhibitor and the second agent is either a vasomodulator, a vasoconstrictor, a vasodilator, or a xanthine compound, the selective cyclooxygenase-2 inhibitor and the vasomodulator are administered as separate dosage forms sequentially or concurrently. Preferably, the xanthine compound is caffeine.

1. Process to Make Nanoparticles

Numerous processes for preparation of nanoparticulate compositions of therapeutic agents are known. Some of these processes use mechanical means, such as milling, to reduce particle size to a nano range, and others precipitate nano-sized particles from solution. Illustrative processes are disclosed in the Patent publications cited below, all incorporated herein by reference.

U.S. Pat. No. 4,826,689 to Violanto & Fischer.

U.S. Pat. No. 5,145,684 to Liversidge et al.

U.S. Pat. No. 5,298,262 to Na & Rajagopalan.

U.S. Pat. No. 5,302,401 to Liversidge et al.

U.S. Pat. No. 5,336,507 to Na & Rajagopalan.

U.S. Pat. No. 5,340,564 to Illig & Sarpotdar.

U.S. Pat. No. 5,346,702 to Na & Rajagopalan.

U.S. Pat. No. 5,352,459 to Hollister et al.

U.S. Pat. No. 5,354,560 to Lovrecich.

U.S. Pat. No. 5,384,124 to Courteille et al.

U.S. Pat. No. 5,429,824 to June.

U.S. Pat. No. 5,510,118 to Bosch et al.

U.S. Pat. No. 5,518,738 to Eickhoff et al.

U.S. Pat. No. 5,503,723 to Ruddy & Eickhoff.

U.S. Pat. No. 5,534,270 to De Castro.

U.S. Pat. No. 5,536,508 to Canal et al.

U.S. Pat. No. 5,552,160 to Liversidge et al.

U.S. Pat. No. 5,560,931 to Eickhoff et al.

U.S. Pat. No. 5,560,932 to Bagchi et al.

U.S. Pat. No. 5,565,188 to Wong et al.

U.S. Pat. No. 5,569,448 to Wong et al.

U.S. Pat. No. 5,571,536 to Eickhoff et al.

U.S. Pat. No. 5,573,783 to Desieno & Stetsko.

U.S. Pat. No. 5,580,579 to Ruddy et al.

U.S. Pat. No. 5,585,108 to Ruddy et al.

U.S. Pat. No. 5,587,143 to Wong.

U.S. Pat. No. 5,591,456 to Franson & Snyder.

U.S. Pat. No. 5,662,883 to Bagchi et al.

U.S. Pat. No. 5,665,331 to Bagchi et al.

U.S. Pat. No. 5,718,919 to Ruddy & Roberts.

U.S. Pat. No. 5,747,001 to Wiedmann et al.

International Publication No. WO 93/25190.

International Publication No. WO 96/24336.

International Publication No. WO 98/35666.

One method of providing suspended particulate celecoxib in a particle size range suitable for practice of the present invention involves a first step of dissolving the celecoxib in a suitable solvent such as ethanol. Preferably the amount of solvent used is kept to a minimum, but must be sufficient to fully dissolve the celecoxib. Preferably a suitable amount of a wetting agent such as polysorbate 80 is also added to the solvent; this can be done before or after, preferably before, addition of the celecoxib. Celecoxib can be added to the ethanol as technical drug, i.e., without the presence of excipients, or in the form of a celecoxib formulation comprising one or more excipients such as diluents, e.g., lactose and/or microcrystalline cellulose, disintegrants, e.g., croscarmellose sodium, binding agents, e.g., polyvinylpyrrolidone, wetting agents, e.g., sodium lauryl sulfate, and lubricants, e.g., magnesium stearate.

In a second step, the resulting solution of celecoxib is added to an aqueous liquid and vigorously agitated, for example by stirring. The volume of the aqueous liquid is much greater than the volume of the celecoxib solution. The effect of the second step is to precipitate celecoxib as a fine suspension in the aqueous liquid. The aqueous liquid can be water and can include other ingredients, such as one or more materials selected from sweetening agents, flavoring agents and coloring agents. The aqueous liquid can be a beverage such as a fruit juice, e.g., apple juice, grape juice, cranberry juice, orange juice, etc.

2. Formulation Process

A drug substance or drug powder prepared according to the above processes or any other process can be administered orally, rectally or parenterally without further formulation, or in simple suspension in water or another pharmaceutically acceptable liquid. Alternatively, the drug substance or drug powder can be directly filled into capsules for oral administration. A composition of the invention can be a substantially homogeneous flowable mass such as a particulate or granular solid or a liquid, or it can be in the form of discrete articles such as capsules or tablets.

In a composition that is a substantially homogeneous flowable mass, single doses are measurably removable using a suitable volumetric measuring device such as a spoon or cup. Suitable flowable masses include, but are not limited to, powders and granules. Alternatively, the flowable mass can be a suspension having the valdecoxib in a solid particulate phase dispersed in a liquid phase, preferably an aqueous phase. In preparing such a suspension, use of a wetting agent such as polysorbate 80 or the like is likely to be beneficial. A suspension can be prepared by dispersing milled valdecoxib in the liquid phase; alternatively the valdecoxib can be precipitated from solution in a solvent such as an alcohol, preferably ethanol. The aqueous phase preferably comprises a palatable vehicle such as water, syrup or fruit juice, for example apple juice.

Although unit dose hard capsule and tablet compositions of the invention can be prepared, for example, by direct encapsulation or direct compression, they preferably are wet granulated prior to encapsulation or compression. Wet granulation, among other effects, densities milled compositions resulting in improved flow properties, improved compression characteristics and easier metering or weight dispensing of the compositions for encapsulation or tableting. The secondary particle size resulting from granulation (i.e., granule size) is not narrowly critical, it being important only that the average granule size preferably is such as to allow for convenient handling and processing and, for tablets, to permit the formation of a directly compressible mixture that forms pharmaceutically acceptable tablets.

The desired tap and bulk densities of the granules are normally about 0.3 g/ml to about 1.0 g/ml.

For tablet formulations, the complete mixture in an amount sufficient to make a uniform batch of tablets is subjected to tableting in a conventional production scale tableting machine at normal compression pressure (for example, applying a force of about 1 kN to about 50 kN in a typical tableting die). Any tablet hardness convenient with respect to handling, manufacture, storage and ingestion may be employed. For 100 mg tablets, hardness is preferably at least 4 kP, more preferably at least about 5 kP, and still more preferably at least about 6 kP. For 200 mg tablets, hardness is preferably at least 7 kP, more preferably at least about 9 kP, and still more preferably at least about 11 kP. The mixture, however, is not to be compressed to such a degree that there is subsequent difficulty in achieving hydration when exposed to gastric fluid.

For tablet formulations, tablet friability preferably is less than about 1.0%, more preferably less than 0.8%, and still more preferably less than about 0.5% in a standard test.

Wet granulation is a preferred method of preparing pharmaceutical compositions of the present invention. In the wet granulation process, any portion of the cyclooxygenase-2 inhibitor or celecoxib that is not to be included in nanoparticulate form (if desired, together with one or more carrier materials) is preferably initially milled or micronized to a desired range of particle sizes such that D₉₀ particle size is greater than 25 μm. The wet granulation process is well known in the art. Impact milling such as pin milling of the drug provides improved blend uniformity to the final composition relative to other types of milling. Cooling of the material being milled, for example, using liquid nitrogen, may be necessary during milling to avoid heating the celecoxib to undesirable temperatures.

The milled or micronized celecoxib, if any, is then blended with the desired amount of celecoxib or cyclooxygenase-2 inhibitor and a vasomodulator in nanoparticulate form (“the nanoparticulate compound”) or in controlled-release, slow-release, programmed-release, timed-release, pulse-release, sustained-release or extended-release form, prepared by any process known in the art as indicated hereinabove. The nanoparticulate compound can be blended with one or more excipients or alternatively the excipients can be added at a later step. For example, in tablet formulations where croscarmellose sodium is employed as a disintegrant, addition of a portion of the croscarmellose sodium during the blending step (providing intragranular croscarmellose sodium) and addition of the remaining portion after the drying step (providing extragranular croscarmellose sodium) can improve disintegration of the tablets produced. In this situation, preferably about 60% to about 75% of the croscarmellose sodium is added intragranularly and about 25% to about 40% of the croscarmellose sodium is added extragranularly. Similarly, for tablet formulations it has been discovered that addition of microcrystalline cellulose after the drying step below (extragranular microcrystalline cellulose) can improve compressibility of the granules and hardness of the tablets prepared from the granules.

3. Excipients

Through selection and combination of excipients, compositions can be provided exhibiting improved performance with respect to efficacy, bioavailability, clearance time, stability, compatibility of valdecoxib and excipients, safety, dissolution profile, disintegration profile and/or other pharmacokinetic, chemical and/or physical properties. The excipients preferably include one or more materials that are water-soluble or water-dispersible and have wetting properties to offset the low aqueous solubility and hydrophobicity of valdecoxib. Where the composition is formulated as a tablet, the combination of excipients selected provides tablets that can exhibit improvement, among other properties, in dissolution and disintegration profiles, hardness, crushing strength and/or friability.

Compositions of the invention can be prepared by any suitable method of pharmacy which includes a step of bringing into association the cyclooxygenase-2 inhibitor, the vasomodulator, and the excipient(s). In general, the compositions are prepared by uniformly and intimately admixing valdecoxib with a liquid or finely divided solid diluent, and then, if necessary, encapsulating or shaping the resulting blend.

Compositions of the invention optionally contain pharmaceutically acceptable excipients other than polyethylene glycol and free radical-scavenging antioxidants. In the case of a solution composition, for example, such excipients can include co-solvents, sweeteners, crystallization inhibitors, preservatives, dispersants, emulsifying agents, etc. Through selection and combination of excipients, compositions can be provided exhibiting improved performance with respect to drug concentration, dissolution, dispersion, emulsification, efficacy, flavor, patient compliance and other properties.

A composition, particularly a solution composition, of the invention optionally comprises one or more pharmaceutically acceptable co-solvents. Non-limiting examples of suitable co-solvents include additional glycols, alcohols, for example ethanol and n-butanol; oleic and linoleic acid triglycerides, for example soybean oil; caprylic/capric triglycerides, for example Miglyol™ 812 of Huls; caprylic/capric mono- and diglycerides, for example Capmul™ MCM of Abitec; polyoxyethylene caprylic/capric glycerides such as polyoxyethylene (8) caprylic/capric mono- and diglycerides, for example Labrasol™ of Gattefosse; propylene glycol fatty acid esters, for example propylene glycol laurate; polyoxyethylene (35) castor oil, for example Cremophor™ EL of BASF; polyoxyethylene glyceryl trioleate, for example Tagat™ TO of Goldschmidt; lower alkyl esters of fatty acids, for example ethyl butyrate, ethyl caprylate and ethyl oleate; and water.

Compositions of the invention suitable for buccal or sublingual administration include, for example, lozenges comprising valdecoxib in a flavored base, such as sucrose, and acacia or tragacanth, and pastilles comprising valdecoxib in an inert base such as gelatin and glycerin or sucrose and acacia.

Liquid dosage forms include suspensions of valdecoxib in a liquid diluent, which is typically aqueous. Such suspensions can contain additional excipients, for example wetting agents, emulsifying and suspending agents, stabilizing agents, thickening agents, and sweetening, flavoring, and perfuming agents.

Compositions of the invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of—and amorphous cellulose (e.g., Rexcel™) and powdered cellulose; calcium carbonate; glycine; bentonite; polyvinylpyrrolidone; and the like. Such diluents, if present, constitute in total about 5% to about 99%, preferably about 10% to about 85%, and more preferably about 20% to about 80%, of the total weight of the composition. The diluent or diluents selected preferably exhibit suitable flow properties and, where tablets are desired, compressibility.

Lactose and microcrystalline cellulose, either individually or in combination, are preferred diluents. Both diluents are chemically compatible with valdecoxib. The use of extragranular microcrystalline cellulose (that is, microcrystalline cellulose added to a wet granulated composition after a drying step) can be used to improve hardness (for tablets) and/or disintegration time. Lactose, especially lactose monohydrate, is particularly preferred. Lactose typically provides compositions having suitable release rates of valdecoxib, stability, pre-compression flowability, and/or drying properties at a relatively low diluent cost. It provides a high density substrate that aids densification during granulation (where wet granulation is employed) and therefore improves blend flow properties.

A composition of the invention optionally comprises one or more pharmaceutically acceptable sweeteners. Non-limiting examples of suitable sweeteners include mannitol, propylene glycol, sodium saccharin, acesulfame K, neotame and aspartame. Alternatively or in addition, a viscous sweetener such as sorbitol solution, syrup (sucrose solution) or high-fructose corn syrup can be used and, in addition to sweetening effects, can also be useful to increase viscosity and to retard sedimentation. Use of sweeteners is especially advantageous in imbibable compositions of the invention, as these can be tasted by the subject prior to swallowing. An encapsulated composition does not typically interact with the organs of taste in the mouth and use of a sweetener is normally unnecessary.

A composition of the invention optionally comprises one or more pharmaceutically acceptable preservatives other than free radical-scavenging antioxidants. Non-limiting examples of suitable preservatives include benzalkonium chloride, benzethonium chloride, benzyl alcohol, chlorobutanol, phenol, phenylethyl alcohol, phenylmercuric nitrate, thimerosal, etc.

Compositions of the invention optionally comprise one or more pharmaceutically acceptable disintegrants as excipients, particularly for tablet formulations. Suitable disintegrants include, either individually or in combination, starches, including sodium starch glycolate (e.g., Explotab™ of PenWest) and pregelatinized corn starches (e.g., National™ 1551, National™ 1550, and Colocorn™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, locust bean, karaya, pectin and tragacanth gums.

Disintegrants may be added at any suitable step during the preparation of the composition, particularly prior to granulation or during a lubrication step prior to compression. Such disintegrants, if present to promote intragastrointestinal dispersion, constitute in total about 0.2% to about 30%, preferably about 0.2% to about 10%, and more preferably about 0.2% to about 5%, of the total weight of the composition.

Optionally, one or more effervescent agents can be used as disintegrants and/or to enhance organoleptic properties of compositions of the invention. When present in compositions of the invention to promote dosage form disintegration, one or more effervescent agents are preferably present in a total amount of about 30% to about 75%, and preferably about 45% to about 70%, for example about 60%, by weight of the composition.

Croscarmellose sodium is a preferred disintegrant for tablet or capsule disintegration, and, if present, preferably constitutes about 0.2% to about 10%, more preferably about 0.2% to about 7%, and still more preferably about 0.2% to about 5%, of the total weight of the composition. Croscarmellose sodium confers superior intragranular disintegration capabilities to granulated compositions of the present invention.

Excipients for tablet compositions of the invention are preferably selected to provide a disintegration time of less than about 30 minutes, preferably about 25 minutes or less, more preferably about 20 minutes or less, and still more preferably about 15 minutes or less, in a standard disintegration assay.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and sodium carboxymethylcellulose (e.g., Tylose™); alginic acid and salts of alginic acid; magnesium aluminum silicate; polyethylene glycol (PEG); guar gum; polysaccharide acids; bentonites; polyvinylpyrrolidone (povidone or PVP), for example povidone K-15, K-30 and K-29/32; polymethacrylates; hydroxypropylmethylcellulose (HPMC); hydroxypropylcellulose (e.g., Klucel™); and ethylcellulose (e.g., Ethocel™). Such binding agents and/or adhesives, if present, constitute in total about 0.5% to about 25%, preferably about 0.75% to about 15%, and more preferably about 1% to about 10%, of the total weight of the composition.

Pregelatinized starch is a preferred binding agent used to impart cohesive properties to a powder blend of valdecoxib and other excipients for granulation of a valdecoxib formulation. Pregelatinized starch, if present, preferably constitutes about 0.5% to about 20%, more preferably about 5% to about 15%, of the total weight of the composition, and facilitates binding of particles in the blend to form granules during wet granulation.

Compositions of the invention optionally comprise one or more pharmaceutically acceptable wetting agents as excipients. Such wetting agents are preferably selected to maintain the valdecoxib in close association with water, a condition that is believed to improve bioavailability of the composition.

Non-limiting examples of surfactants that can be used as wetting agents in compositions of the present invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefosse), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the composition.

Wetting agents that are anionic surfactants are preferred. Sodium lauryl sulfate is a particularly preferred wetting agent. Sodium lauryl sulfate, if present, constitutes about 0.25% to about 7%, more preferably about 0.4% to about 4%, and still more preferably about 0.5% to about 2%, of the total weight of the composition.

Compositions of the invention optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; polyethylene glycols (e.g., Carbowax™ 4000 and Carbowax™ 6000); sodium oleate; sodium lauryl sulfate; and magnesium lauryl sulfate. Such lubricants, if present, constitute in total about 0.1% to about 10%, preferably about 0.2% to about 8%, and more preferably about 0.25% to about 5%, of the total weight of the composition.

Glidants can be used to promote powder flow of a solid formulation. Suitable glidants include colloidal silicon dioxide, starch, talc, tribasic calcium phosphate, powdered cellulose and magnesium trisilicate. Colloidal silicon dioxide is particularly preferred.

Magnesium stearate is a preferred lubricant used, for example, to reduce friction between the equipment and granulated mixture during compression of tablet formulations.

Suitable anti-adherents include talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0.1% to about 10%, more preferably about 0.25% to about 5%, and still more preferably about 0.5% to about 2%, of the total weight of the composition.

Additionally, compositions of the invention optionally comprise one or more pharmaceutically acceptable buffering agents, flavoring agents, colorants, stabilizers and/or thickeners. Buffers can be used to control pH of a formulation and can thereby modulate drug solubility. Flavoring agents can enhance patient compliance by making the composition more palatable, particularly in the case of an imbibable composition, and colorants can provide a product with a more aesthetic and/or distinctive appearance. Non-limiting examples of suitable colorants include D&C Red No. 33, FD&C Red No. 3, FD&C Red No. 40, D&C Yellow No. 10, and C Yellow No. 6.

4. General Dose and Treatment Issues

The present invention is further directed to a therapeutic method of treating a condition or disorder where treatment with a COX-2 inhibitory drug is indicated, the method comprising oral administration of a composition of the invention to a subject in need thereof. The dosage regimen to prevent, give relief from, or ameliorate the condition or disorder preferably corresponds to once-a-day or twice-a-day treatment, but can be modified in accordance with a variety of factors. These include the type, age, weight, sex, diet and medical condition of the subject and the nature and severity of the disorder. Thus, the dosage regimen actually employed can vary widely and can therefore deviate from the preferred dosage regimens set forth above.

Initial treatment can begin with a dose regimen as indicated above. Treatment is generally continued as necessary over a period of several weeks to several months or years until the condition or disorder has been controlled or eliminated. Subjects undergoing treatment with a composition of the invention can be routinely monitored by any of the methods well known in the art to determine effectiveness of therapy. Continuous analysis of data from such monitoring permits modification of the treatment regimen during therapy so that optimally effective doses are administered at any point in time, and so that the duration of treatment can be determined. In this way, the treatment regimen and dosing schedule can be rationally modified over the course of therapy so that the lowest amount of the composition exhibiting satisfactory effectiveness is administered, and so that administration is continued only for so long as is necessary to successfully treat the condition or disorder.

Definitions

The term “cyclooxygenase-2 inhibitor” denotes a compound able to inhibit cyclooxygenase-2 without significant inhibition of cyclooxygenase-1. Preferably, it includes compounds that have a selective cyclooxygenase-2 ICSO of less than about 0.2 micromolar, and also have a selectivity ratio of cyclooxygenase-2 inhibition over cyclooxygenase-1 inhibition of at least 50, and more preferably of at least 100. Even more preferably, the compounds have a cyclooxygenase-1 IC₅₀ of greater than about 1 micromolar, and more preferably of greater than 10 micromolar.

Derivatives are intended to encompass any compounds which are structurally related to the cyclooxygenase-2 inhibitors or which possess the substantially equivalent biologic activity. By way of example, such inhibitors may include, but are not limited to, prodrugs thereof.

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical. Where used, either alone or within other terms such as “haloalkyl”, “alkylsulfonyl”, “alkoxyalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like. The term “alkenyl” embraces linear or branched radicals having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl. The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like. The terms “alkenyl”, “lower alkenyl”, embrace radicals having “cis” and “trans” orientations, or alternatively, “E” and “Z” orientations. The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl. The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl, and cyclohexenyl. The term “halo” means halogens such as fluorine, chlorine, bromine or iodine. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Examples of haloalkyl radicals include fluoromethyl, difluoromethyl, trifluoromethyl, chloromethyl, dichloromethyl, trichloromethyl, trichloromethyl, pentafluoroethyl, heptafluoropropyl, difluorochloromethyl, dichlorofluoromethyl, difluoroethyl, difluoropropyl, dichloroethyl and dichloropropyl. The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy. The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl. The term “heterocyclyl” embraces saturated, partially unsaturated and unsaturated heteroatom-containing ring-shaped radicals, where the heteroatoms may be selected from nitrogen, sulfur and oxygen. Examples of saturated heterocyclyl radicals include saturated 3 to 6-membered heteromonocylic group containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclyl radicals include dihydrothiophene, dihydropyran, dihydrofuran and dihydrothiazole. The term “heteroaryl” embraces unsaturated heterocyclyl radicals. Examples of unsaturated heterocyclyl radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclyl group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclyl group containing 1 to 2 oxygen atoms and 1 to 3 nitrogen atoms (e.g. benzoxazolyl, benzoxadiazolyl, etc.); unsaturated 3 to 6-membered heteromonocyclic group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclyl group containing 1 to 2 sulfur atoms and 1 to 3 nitrogen atoms (e.g., benzothiazolyl, benzothiadiazolyl, etc.) and the like. The term also embraces radicals where heterocyclyl radicals are fused with aryl radicals. Examples of such fused bicyclic radicals include benzofuran, benzothiophene, and the like. Said “heterocyclyl group” may have 1 to 3 substituents such as alkyl, hydroxyl, halo, alkoxy, oxo, amino and alkylamino. The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio. The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl. The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl. The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals. The terms “sulfamyl”, “aminosulfonyl” and “sulfonamidyl” denote NH₂O₂S—. The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl. The term “carbonyl”, whether used alone or with other terms, such as “alkoxycarbonyl”, denotes —(C═O)—. The term “aroyl” embraces aryl radicals with a carbonyl radical as defined above. Examples of aroyl include benzoyl, naphthoyl, and the like and the aryl in said aroyl may be additionally substituted. The terms “carboxy” or “carboxyl”, whether used alone or with other terms, such as “carboxyalkyl”, denotes —CO₂H. The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl. The term “alkoxycarbonyl” means a radical containing an alkoxy radical, as defined above, attached via an oxygen atom to a carbonyl radical. More preferred are “lower alkoxycarbonyl” radicals with alkyl porions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl. The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl. The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, halkoalkyl and haloalkoxy. The terms benzyl and phenylmethyl are interchangeable. The term “heterocyclylalkyl” embraces saturated and partially unsaturated heterocyclyl-substituted alkyl radicals, such as pyrrolidinylmethyl, and heteroaryl-substituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, halkoalkyl and haloalkoxy. The term “aralkoxyl” embraces aralkyl radicals attached through an oxygen atom to other radicals. The term “aralkoxyalkyl” embraces aralkoxy radicals attached through an oxygen atom to an alkyl radical. The term “aralkylthio” embraces aralkyl radicals attached to a sulfur atom. The term “aralkylthioalkyl”, embraces aralkylthio radicals attached through a sulfur atom to an alkyl radical. The term “aminoalkyl” embraces alkyl radicals substituted with one or more amino radicals. More preferred are “lower aminoalkyl” radicals. Examples of such radicals include aminomethyl, aminoethyl, and the like. The term “alkylamino” denotes amino groups which have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like. The term “arylamino” denotes amino groups which have been substituted with one or two aryl radicals, such as N-phenylamino. The “arylamino” radicals may be further substituted on the aryl ring portion of the radical. The term “aralkylamino” embraces aralkyl radicals attached through an amino nitrogen atom to other radicals. The terms “N-arylaminoalkyl” and “N-aryl-N-alkyl-aminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl. The term “aminocarbonyl” denotes an amide group of the formula —C(═O)NH₂. The term “alkylaminocarbonyl” denotes an aminocarbonyl group which has been substituted with one or two alkyl radicals on the amino nitrogen atom. Preferred are “N-alkylaminocarbonyl” “N,N-dialkylaminocarbonyl” radicals. More preferred are “lower N-alkylaminocarbonyl” “lower N,N-dialkylaminocarbonyl” radicals with lower alkyl portions as defined above. The term “alkylaminoalkyl” embraces radicals having one or more alkyl radicals attached to an aminoalkyl radical. The term “aryloxyalkyl”, embraces radicals having an aryl radical attached to an alkyl radical through a divalent oxygen atom. The term “arylthioalkyl”, embraces radicals having an aryl radical attached to an alkyl radical through a divalent sulfur atom.

An amount sufficient to “substantially inhibit drug crystallization and/or precipitation” herein means an amount sufficient to prevent, slow, inhibit or delay precipitation of drug from solution and/or to prevent, slow, inhibit or delay formation of crystalline drug particles from dissolved drug particles.

A polymer component such as HPMC is “present in the capsule wall” or is a “capsule wall component” as described herein if the polymer is (a) dispersed or mixed together with any other capsule wall component (s), (b) the only capsule wall component, or (c) present as a coating on the outside or inside of the capsule wall.

An “intimate association” in the present context includes, for example, celecoxib admixed with the crystallization inhibitor, celecoxib embedded or incorporated in the crystallization inhibitor, celecoxib forming a coating on particles of the crystallization inhibitor or vice versa, and a substantially homogeneous dispersion of celecoxib throughout the crystallization inhibitor. The term “substantially homogeneous” herein with reference to a composite or pharmaceutical composition that comprises multiple components means that the components are sufficiently mixed such that individual components are not present as discrete layers and do not form concentration gradients within the composition.

D₉₀ is a diameter such that 90% by weight of the particles are smaller than this diameter in their longest dimension.

This detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

### EXAMPLES

Biological Assays

The utility of the combinations of the present invention can be shown by the following assays. These assays are performed in vitro and in animal models essentially using procedures recognized to show the utility of the present invention.

Rat Carrageenan Foot Pad Edema Test

The carrageenan foot edema test is performed with materials, reagents and procedures essentially as described by Winter, et al., (Proc. Soc. Exp. Biol. Med., 111, 544 (1962)). Male Sprague-Dawley rats are selected in each group so that the average body weight is as close as possible. Rats are fasted with free access to water for over sixteen hours prior to the test. The rats are dosed orally (1 mL) with compounds suspended in vehicle containing 0.5% methylcellulose and 0.025% surfactant, or with vehicle alone. One hour later a subplantar injection of 0.1 mL of 1% solution of carrageenan/sterile 0.9% saline is administered and the volume of the injected foot is measured with a displacement plethysmometer connected to a pressure transducer with a digital indicator. Three hours after the injection of the carrageenan, the volume of the foot is again measured. The average foot swelling in a group of drug-treated animals is compared with that of a group of placebo-treated animals and the percentage inhibition of edema is determined (Otterness and Bliven, Laboratory Models for Testing NSAIDS, in Non-steroidal Anti-Inflammatory Drugs, (J. Lombardino, ed. 1985)). The % inhibition shows the % decrease from control paw volume determined in this procedure.

Rat Carrageenan-induced Analgesia Test

The analgesia test using rat carrageenan is performed with materials, reagents and procedures essentially as described by Hargreaves, et al., (Pain, 32, 77 (1988)). Male Sprague-Dawley rats are treated as previously described for the Carrageenan Foot Pad Edema test. Three hours after the injection of the carrageenan, the rats are placed in a special plexiglass container with a transparent floor having a high intensity lamp as a radiant heat source, positionable under the floor. After an initial twenty minute period, thermal stimulation is begun on either the injected foot or on the contralateral uninjected foot. A photoelectric cell turns off the lamp and timer when light is interrupted by paw withdrawal. The time until the rat withdraws its foot is then measured. The withdrawal latency in seconds is determined for the control and drug-treated groups, and percent inhibition of the hyperalgesic foot withdrawal determined.

Evaluation of COX-1 and COX-2 Activity in vitro

The compounds of this invention exhibit inhibition in vitro of COX-2. The COX-2 inhibition activity of the compounds of this invention illustrated in the Examples is determined by the following methods.

a. Preparation of Recombinant COX Baculoviruses

A 2.0 kb fragment containing the coding region of either human or murine COX-1 or human or murine COX-2 is cloned into a BamH1 site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×10 e8) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (10E7-10E8 pfu/ml) stocks of virus are prepared. For large scale production, SF9 insect cells are infected in 10 liter fermentors (0.5×10⁶/ml) with the recombinant baculovirus stock such that the multiplicity of infection is 0.1. After 72 hours the cells are centrifuged and the cell pellet homogenized in Tris/Sucrose (50 mM: 25%, pH 8.0) containing 1% 3-[(3-cholamidopropyl)dimethylammonio]-1-propanesulfonate (CHAPS). The homogenate is centrifuged at 10,000× G for 30 minutes, and the resultant supernatant is stored at −80° C. before being assayed for COX activity.

b. Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 micromolar). Compounds are pre-incubated with the enzyme for 10-20 minutes prior to the addition of arachidonic acid. Any reaction between the arachidonic acid and the enzyme is stopped after ten minutes at 370° C./room temperature by transferring 40 microliter of reaction mix into 160 microliter ELISA buffer and 25 μM indomethacin. The PGE2 formed is measured by standard ELISA technology (Cayman Chemical).

### Example 1

Six celecoxib solution formulations SF-1 to SF-6 were prepared having components as shown in Table 3. In each case the solvent liquid consisted of PEG-400, either alone (SF-1) or together with a free radical-scavenging antioxidant (SF-2 to SF-6). Celecoxib was present in solution at a concentration of 50 mg/g in all formulations. Antioxidant amounts are shown as % weight/weight.

TABLE 4
Composition of celecoxib solution formulations
SF-1 to SF-6

| Formulation | Components |
|---|---|
| SF-1 | Celecoxib, PEG-400 |
| SF-2 | Celecoxib, PEG-400, 0.1% vitamin E |
| SF-3 | Celecoxib, PEG-400, 0.1% BHA |
| SF-4 | Celecoxib, PEG-400, 0.1% BHT |
| SF-5 | Celecoxib, PEG-400, 0.1% propyl |
| | gallate |
| SF-6 | B. Celecoxib, PEG-400. 0.05% BHA, 0.05% BHT |

### Example 2

A gradient HPLC assay was used to determine impurities in celecoxib solution formulations SF-1 to SF-6 of Example 1 after storage at various temperatures for different periods of time. Solution formulation samples were drawn and were dissolved in methanol to obtain a celecoxib concentration of about 0.4 to about 0.5 mg/ml prior to injection. Chromatographic conditions were as follows: (a) flow rate: 1 ml/min.; (b) detection: UV 254 nm; (c) injection volume: 10 microliter; (d) column: 5 micrometer Supercosil, LC-DP, 250×4.6 mm; (e) column temperature: 40° C.; (f) mobile phase A: 10 mM NH₄AC or KH₂PO₄, pH 3; (g) mobile phase B: 100% acetonitrile; (h) running time: 45 minutes. Data are shown in Tables 5 and 6.

TABLE 5
Impurity level (%) in formulations SF-1 to
SF-5 following storage

| Formula- | days stored at 70° C. | | | | | | | |
|---|---|---|---|---|---|---|---|---|
| tion | 9 | 14 | 16 | 20 | 28 | 33 | 35 | 90 |
| SF-1 | 2.9 | | 3.7 | | 7.6 | | 12.6 | |
| SF-2 | | 0.02 | | 0.02 | | 0.02 | | 2.8 |
| SF-3 | | 0.02 | | 0.02 | | 0.02 | | 0.09 |
| SF-4 | | 0.03 | | 0.04 | | 0.06 | | 0.30 |
| SF-5 | | ND | | ND | | ND | | 0.15 |

ND = None detected

TABLE 6
Impurity level (%) in formulations SF-1, SF-2,
SF-5 and SF-6 following storage at different temperatures

| Formula- | | Temperature | | | |
|---|---|---|---|---|---|
| tion | Days | 50° C. | 40° C. | 25° C. | 4° C. |
| SF-1 | 0 | 0.00 | 0.00 | 0.00 | 0.00 |
| | 7 | 0.09 | | | |
| | 21 | 4.12 | 0.11 | 0.00 | |
| | 31 | 6.25 | | | 0.00 |
| | 74 | 7.83 | 5.40 | 0.08 | 0.00 |
| | 131 | 7.85 | 6.87 | 0.44 | 0.00 |
| SF-2 | 0 | 0.00 | 0.00 | 0.00 | 0.00 |
| | 7 | 0.00 | | | |
| | 21 | 0.02 | 0.00 | 0.00 | |
| | 31 | 0.01 | | | 0.00 |
| | 74 | 0.06 | 0.02 | 0.00 | 0.00 |
| | 131 | 0.07 | 0.01 | 0.00 | 0.00 |
| SF-5 | 0 | 0.00 | 0.00 | 0.00 | 0.00 |
| | 7 | 0.02 | | | |
| | 21 | 0.05 | 0.03 | 0.02 | |
| | 31 | 0.05 | | | 0.00 |
| | 74 | 0.15 | 0.11 | 0.03 | 0.00 |
| | 131 | 0.20 | 0.09 | 0.02 | 0.00 |
| SF-6 | 0 | 0.00 | 0.00 | 0.00 | 0.00 |
| | 7 | 0.00 | | | |
| | 21 | 0.01 | 0.01 | 0.00 | |
| | 31 | 0.01 | | | 0.00 |
| | 74 | 0.03 | 0.02 | 0.01 | 0.00 |
| | 131 | 0.06 | 0.01 | 0.00 | 0.00 |

The data in Tables 5 and 6 indicate that the presence of a small amount of a free radical-scavenging antioxidant such as vitamin E, butyl gallate, BHA or BHT greatly improves chemical stability of celecoxib dissolved in PEG-400 by comparison with compositions comprising no such antioxidant.

### Example 4

Solution formulation SF-1 of Example 1 was bubbled with ethylene oxide, a putative source of free radicals, for 15 minutes, and was then stored at 70° C. for 10 days. After storage, the formulation was analyzed for the presence of impurities. Addition compounds detected therein were isolated by reversed-phase, semi-preparative HPLC. A 20×250 mm Kromasil C18 column was employed with either an isocratic or gradient, acetonitrile-aqueous trifluoroacetic acid mobile phase. Detection was accomplished at 254 nm. Pooled fractions containing individual addition compounds, herein referred to as Peak 1, Peak 2 and Peak 3 addition compounds, were concentrated, desalted and reduced in chemical noise-causing components by trapping on a 7×300 mm Hamilton PRP-1 column. The eluent from the trapping column containing the individual addition compounds was freeze-dried to yield the final isolates. Peak 1 addition compound was 99% pure and Peak 2 addition compound was 99% pure by analytical HPLC. Peak 3 addition compound was 81% pure by analytical HPLC.

Analytical HPLC was also used to collect analytical scale peak cuts for mass spectrometric analysis on a PE Sciex Q-Star QQ-TOF mass spectrometer. Survey and product ion scans, as well as high resolution mass measurements for empirical formula determination were acquired in μESI (micro-electrospray ionization) mode. High resolution mass spectral information on Peak 1 and Peak 2 addition compounds were obtained on a Finnigan MAT-900ST mass spectrometer operating in μESI mode. Accurate mass measurement for Peak 1 addition compound was carried out by linear E-scan peak matching at a resolution of 7,400 (m/micrometer 10% valley definition) using the reference ions from PEG-400, (C₂H₄O)₉H₂ONa at 437.23627 and (C₂H₄O)₁₀H₂ONa at 481.26248 daltons, respectively, to match against the sample pseudo-molecular ion. Accurate mass measurement for Peak 2 addition compound was carried out by linear E-scan peak matching at a resolution of 7,100 (m/micrometer 10% valley definition) using the reference ions from PEG-400 (C₂H₄O)₈H₂ONa at 393.21005 and (C₂H₄O)₉H₂ONa at 437.23627 daltons, respectively, to match against the sample pseudo-molecular ion.

NMR samples were prepared in a nitrogen glove box and dissolved in 150 microliter dimethyl sulfoxide-delta₆. Data were acquired on a Varian INOVA 400 NMR spectrometer operating at a proton frequency of 399.80 MHz, and equipped with a Nalorac inverse geometry, micro-gradient probe. Experiments were used directly from the vendor's standard library with no modifications.

Peak 1

Celecoxib and Peak 1 addition compound were individually mounted on gold-coated microscope slides for IR and Raman analyses. Micro-IR specular reflectance data were collected from 4000→650 cm⁻¹ at 4-cm⁻¹ resolution on a Nicolet 760 spectrometer equipped with a liquid nitrogen cooled MCT detector. Sensitivity, expressed as instrument gain, was 8. Data were processed as a Fourier transform utilizing a Happ-Genzel apodization function and plotted as % transmittance vs. frequency. The final spectra were the sum of 200 individual scans. Micro-Raman data were collected from 3700→100 cm⁻¹on a Nicolet 960 FT-Raman spectrometer, equipped with a liquid nitrogen cooled germanium detector. Sensitivity, expressed as instrument gain, was 64. Data were processed as a Fourier transform utilizing a Happ-Genzel apodization function and plotted as absorbance vs. frequency. The final spectra were the sum of 10,000 individual scans.

The molecular weight of Peak 1 addition compound was found to be 469 daltons, 88 daltons heavier than celecoxib and indicative of addition of two ethanolic moieties. The molecular weight was confirmed by high resolution peak matching, of an analytical peak cut, as 469.12831 daltons, within 0.2 ppm of theory for C₂₁H₂₂F₃N₃O₄S. The accurate mass of Peak 1 addition compound, less the ionizing proton, was measured as 469.12826 daltons. The empirical formula for best fit using the valence rules was C₂₁H₂₂F3N₃O₄S and within 0.1 ppm in mass from theory, thus confirming the molecular weight of this product. Peak 1 addition compound is believed to have the structure (XVII):

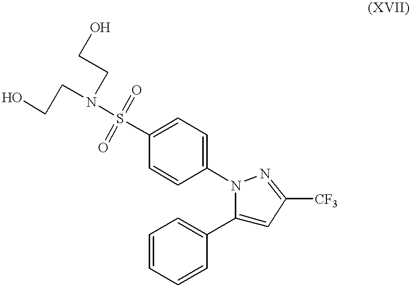

NMR analysis of Peak 1 addition compound produced similar data to those for the bulk drug. A major difference existed in the absence of the —SO₂NH₂ protons, and the inclusion of resonances consistent with the presence of two —CH₂CH₂OH functionalities. The methylene protons and carbons exhibited distinct chemical shifts that are consistent with the proposed structure.

The IR and Raman spectra of celecoxib and Peak 1 addition compound are very similar, indicating that the bulk of the structure is the same as that of celecoxib. Several spectral differences, however, between the two molecules are evident. The two N-H stretching vibrations in the spectrum of celecoxib at 3236 and 3342 cm⁻¹ are missing in the data for Peak 1 addition compound, indicating the amino group present in celecoxib is not present in Peak 1 addition compound. The N—H vibrations in the IR spectrum for celecoxib are replaced by an intense, broad absorbance centered at 3430 cm⁻¹ in the analogous data for Peak 1 addition compound. This broad band is typical of an O—H stretch, but is much too intense to result from a single hydroxyl group, indicating that Peak 1 addition compound possesses at least two OH groups, in place of the NH₂ group present in celecoxib. Another major spectral difference between the vibrational spectra for celecoxib and Peak 1 addition compound are the presence of Raman C—H stretching vibrational bands for Peak 1 addition compound at 2967 and 2991 cm⁻¹ that are not present in the analogous data for celecoxib. These differences indicate the presence of additional CH₂ groups in the addition compound, compared to celecoxib. Both the IR and Raman data are consistent with the proposed structure.

The compound having the structure (V) is believed to be new and is useful as an analytical marker, for example in detecting stability of celecoxib in formulations where the celecoxib is exposed to polyethylene glycol or ethylene oxide.

Peak 2

The molecular weight of Peak 2 addition compound was found to be 425 daltons, 44 daltons heavier than celecoxib and indicative of the addition of one ethanolic moiety. The molecular weight was confirmed by high resolution peak matching, of an analytical peak cut, as 425.10239 daltons, within 0.9 ppm of theory for C₁₉H₁₈F₃N₃O₃S. The accurate mass of Peak 2 addition compound, less the ionizing proton, was measured as 425.10168 daltons. The empirical formula for best fit using the valence rules was C₁₉H₁₈F₃N₃O₃S and within 1.0 ppm in mass from theory, thus confirming the molecular weight of this compound. Peak 2 addition compound is believed to have the structure (XVIII):

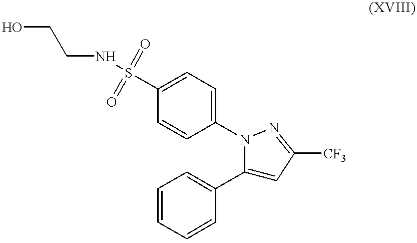

The NMR data for Peak 2 addition compound were similar to those for Peak 1 addition compound in that this isolate also exhibited the —CH₂CH₂OH functionality, but proton integrations identified the presence of only one ethanol substituent. The presence of an —NH— group was also apparent in the proton spectrum. The proton and carbon chemical shifts were in accordance with the proposed structure.

The compound having the structure (VI) is believed to be new and is useful as an analytical marker, for example in detecting stability of celecoxib in formulations where the celecoxib is exposed to polyethylene glycol or ethylene oxide.

Peak 3

Peak 3 addition compound was present in insufficient concentration for an adequate isolate to be obtained for spectroscopic analysis.

### Example 4

Three celecoxib (10 mg/g) solutions (with methanol as solvent), one containing no peroxide (Si), one containing 150 ppm hydrogen peroxide (S2), and one containing 150 ppm t-butyl-peroxide (S3), were prepared. HPLC analysis, as described in Example 2, was performed to determine the presence or absence of impurities following storage at different temperatures for various periods of time (Table 7).

TABLE 7
Chemical stability of celecoxib solutions S1-S3

| | | Total impurity | | |
|---|---|---|---|---|
| Solu- | | level (%) | | |
| tion | Time | 4° C. | 25° C. | 50° C. |
| S1 | 0 | 0.15 | 0.15 | 0.15 |
| | 1 week | 0.15 | 0.15 | 0.54 |
| | 2 weeks | | 0.14 | 1.57 |
| | 3 weeks, then 3 days | | | 2.40 |
| | at 70° C. | | | |
| S2 | 0 | 0.15 | 0.15 | 0.15 |
| | 1 week | 0.15 | 0.15 | 0.46 |
| | 2 weeks | | 0.14 | 0.94 |
| | 3 weeks, then 3 days | | | 1.60 |
| | at 70° C. | | | |
| S3 | 0 | 0.15 | 0.15 | 0.15 |
| | 1 week | 0.15 | 0.15 | 0.33 |
| | 2 weeks | | 0.13 | 0.92 |
| | 3 weeks, then 3 days | | | 2.00 |
| | at 70° C. | | | |

These data indicate that the presence of hydrogen peroxide or t-butyl-peroxide at a concentration of 150 ppm does not affect celecoxib stability in methanol. These data are consistent with the conclusion that chemical instability in a system comprising an aminosulfonyl-comprising drug, for example celecoxib, and a polyethylene glycol, is not peroxide-mediated.

### Example 5

Two celecoxib solution formulations, SF-7, and SF-8, and two vehicle (placebo) solution formulations, SF-9 and SF-10, were prepared having components shown in Table 8.

TABLE 8
Composition (mg) of solution formulations SF-7
to SF-10

| Component | SF-7 | SF-8 | SF-9 | SF-10 |
|---|---|---|---|---|
| Celecoxib | 200 | 200 | | |
| Water USP | 26 | 26 | 26 | 26 |
| HPMC (E5) | 38 | | 38 | |
| Ethanol | 113 | 100 | 113 | 100 |
| PEG-400 | 271 | 322 | 271 | 322 |
| Polyvinylpyrrol | 47 | 47 | 47 | 47 |
| idone | | | | |
| Polysorbate 80 | 217 | 217 | 217 | 217 |
| Tromethamine | 26 | 26 | 26 | 26 |
| Oleic acid | 61 | 61 | 61 | 61 |
| Propyl gallate | 1 | 1 | 1 | 1 |
| NF | | | | |
| Total | 1000 | 1000 | 800 | 800 |

After storage for 90 days at different temperatures, the fraction of the initial 1 mg/g propyl gallate remaining in each formulation was measured via gradient HPLC. Samples of all formulations were dissolved in methanol to obtain a concentration of about 10 microgram/ml propyl gallate prior to injection. Chromatographic conditions were as follows: (a) flow rate: 1 ml/min.; (b) detection: UV 254 nm; (c) injection volume: 15 microliter; (d) column: 3.5 micrometer Zorbax XBD-C8, 50×4.6 mm; (e) column temperature: 25° C.; (f) mobile phase A: 0.1% TFA in water; (g) mobile phase B: 0.1% TFA in acetonitrile; (h) running time: 16 minutes. Data are shown in Table 9.

TABLE 9
Loss of propyl gallate in solution formulations
SF-7 to SF-10 after storage for 90 days

| | |
|---|---|
| | Propyl gallate (% of |
| Temperature | theoretical) remaining |

| (° C.) | SF-7 | SF-8 | SF-9 | SF-10 |
|---|---|---|---|---|
| 4 | 87 | 104 | 108 | 126 |
| 25 | 42 | 74 | 36 | 66 |
| 40 | 10 | 33 | 10 | 24 |
| 50 | 0 | 13 | 0 | 19 |
| 70 | 0 | 0 | 0 | 7 |

These data indicate that, in formulations comprising an aminosulfonyl-comprising drug (celecoxib in the present example) and in those without such a drug, propyl gallate is consumed at a substantially equal rate over 90 days. Moreover, the rate of consumption is temperature dependent with increasing rate as temperature increases. These results suggest that the free radical-scavenging antioxidant is consumed via a non drug-mediated mechanism, and support the present theory that drug stabilization results from an interaction between polyethylene glycol degradation products and the free radical-scavenging antioxidant.

### Example 6

A celecoxib solution formulation, SF-11, was prepared having the composition shown in Table 10.

TABLE 10
Composition (mg/g) of celecoxib solution
formulation SF-11.

| Component | SF-11 |
|---|---|
| Celecoxib | 200 |
| Water USP | 26 |
| HPMC (E5) | 38 |
| Ethanol | 113 |
| PEG 400 | 271 |
| PVP | 47 |
| Polysorbate 80 | 217 |
| Tromethamine | 26 |
| Oleic acid | 61 |
| Propyl gallate | 1 |
| NF | |
| Total | 1000 |

One gram of formulation SF-11 was individually placed into each of several hard gelatin capsules (Capsugel) to form Test Composition 1.

A celecoxib suspension for comparative purposes was prepared as follows:

A. Tween™ 80, 5.0 g, was placed in a volumetric flask.

B. Ethanol was added (to 100 ml) to form a mixture and the mixture was swirled to form a uniform solution.

C. A 5 ml aliquot of the uniform solution was transferred to a fresh 100 ml bottle containing 200 mg celecoxib, to form a premix.

D. Apple juice, 75 ml, was added to the premix to form an intermediate celecoxib suspension.

E. The intermediate celecoxib suspension was left to stand for 5 minutes, and was then shaken to form a celecoxib suspension for comparative purposes.

Bioavailability parameters resulting from administration to human subjects of one capsule of Test Composition 1, in comparison with the above-described celecoxib suspension and with a commercial 200 mg celecoxib capsule (Celebrex® of Pharmacia Corporation), were evaluated as part of a 24 subject, randomized, four period, balanced, crossover study. Celecoxib dose was 200 mg in each treatment. Study duration was approximately 15 days and subjects were randomly given one dosage form on days 1, 5, 9 and 12; administration of each dose was preceded by an 8 hour fasting period and was accompanied by 180 ml of water. Plasma blood levels for each subject were measured at pre-dose and at 15, 30, 45 minutes and 1, 1.5, 2, 3, 4, 6, 8, 12 and 24 hours after dosage administration. Maximum blood serum concentration of celecoxib (Cₘₐₓ), time to reach that concentration (Tₘₐₓ) and area under the curve (AUC, a measure of overall bioavailability) were calculated from the data in accordance with standard procedure in the art. As shown in Table 11, ingestion of Test Composition 1 resulted in a Cₘₐₓ more than 2.5 times greater than that resulting from ingestion of the comparative celecoxib suspension or the Celebrex® capsule. Ingestion of Test Composition 1 also resulted in an AUC 43% greater than that resulting from ingestion of the celecoxib suspension and a Tₘₐₓ substantially similar to that resulting from ingestion of the suspension.

TABLE 11
In vivo bioavailability of Test Composition 1
in human subjects

| | Celebrex ® | Celecoxib | Test |
|---|---|---|---|
| Parameter | capsule | suspension | Composition 1 |
| Cₘₐₓ (ng/ml) | 621 | 804 | 2061 |
| Tₘₐₓ (hr) | 2.15 | 0.97 | 1.03 |
| AUC | 5060 | 4892 | 7593 |
| (ng/ml) *hr | | | |

### Example 7

Solubility of celecoxib and valdecoxib was determined in each of several different solvent liquids as shown in Table 12, below. To determine solubility, a solid sample consisting of a known amount, typically about 50 mg, of celecoxib or valdecoxib powder was weighed into a test tube. Aliquots of a solvent liquid were then added dropwise in approximately 100 mg increments to the solid sample. The resulting mixture was vortexed and/or sonicated between aliquot additions. Aliquots of solvent liquid were added until the solvent liquid was clear, indicating that the sample was completely dissolved. Ranges in Table 12 indicate that the solubility of celecoxib or valdecoxib is between the values given but has not been more precisely determined. Solubility values preceded by the < symbol denote that, at the particular concentration shown, the mixture was still cloudy, i.e., not all of the drug was fully in dissolved form.

TABLE 12
Solubility of celecoxib and valdecoxib in
various solvent liquids

| | Solubility | Solubility |
|---|---|---|
| | of | of |
| | celecoxib | valdecoxib |
| Solvent liquid | (mg/g) | (mg/g) |
| Propylene glycol | 23 − 41 | 10 − 20 |
| Ethyl caprylate | 25 | |
| Propylene glycol laurate | 18 | 22 |
| Labrasol ™ ¹ | 64 | 34 |
| Propylene glycol | 58 | 42 |
| laurate/Labrasol ™ 1:1 w/w | | |
| Capmul ™ MCM² | 19 − 21 | 13 |
| Miglyol ™ 812³ | 6 − 12 | |
| Tagat ™ TO⁴ | 24 − 40 | 23 |
| Tagat ™ TO/Capmul ™ MCM 1:1 w/w | 34 − 52 | 24 |
| Polyethylene glycol 400 | 304 | 50 − 85 |
| Polyethylene glycol 400/water | 6 | 13 |
| 2:1 w/w | | |
| Polyethylene glycol 400/water | <1 | 1 |
| 1:1 w/w | | |
| Diethylene glycol monoethyl | 350 | 120 |
| ether (DGME) | | |
| DGME/water 2:1 w/w | 42 | 32 |
| DGME/water 1:1 w/w | 3 | 6 |
| Labrasol ™ /DGME/propylene | 313 − 325 | |
| glycol laurate 45:45:10 w/w | | |
| Labrasol ™ /DGME/propylene | 288 − 297 | 130 |
| glycol laurate 40:40:20 w/w | | |
| Labrasol ™ /DGME/propylene | 266 | |
| glycol laurate 35:35:30 w/w | | |
| Labrasol ™ /DGME 1:1 w/w | 335 | |
| Tagat ™ /Capmul ™ MCM/DGME | 212 | |
| 35:35:30 w/w | | |
| Tagat ™ /Capmul ™ MCM/DGME | 274 | |
| 58:12:30 w/w | | |
| Tetraethylene glycol dimethyl | 188 | |
| ether | | |
| Triethylene glycol monoethyl | 170 | |
| ether | | |
| Polysorbate 80 | 73 | |
| Arlacel ™ 186⁵ | 13 | |
| Cremophor ™ EL⁶ | 36 | |

¹Labrasol ™ = polyoxyethylene (8) caprylic/capric glycerides
²Capmul ™ MCM = caprylic/capric mono- and diglycerides
³Miglyol ™ 812 = caprylic/capric triglycerides
⁴Tagat ™ TO = polyoxyethylene glyceryl trioleate
⁵Arlacel ™ 186 = glyceryl monooleate
⁶Cremophor ™ EL = polyoxyethylene (35) castor oil

The data in Table 12 illustrate advantages of the glycol ether solvent DGME for preparation of orally deliverable solutions by comparison with glycol solvents such as propylene glycol and polyethylene glycol, that are known in prior art for preparing parenteral solutions of selective COX-2 inhibitory drugs. For example, solubility of celecoxib in DGME has been determined to be about 304 mg/g, by contrast with solubility of the same drug in propylene glycol, which is only about 23-41 mg/g. A similar approximately tenfold advantage in solubility is shown for DGME over propylene glycol in the case of valdecoxib.

Although the solubility advantage of DGME over polyethylene glycol 400 (PEG-400) as a solvent for celecoxib is less pronounced, a major advantage is seen for DGME when water is added to the solvent liquid. Solubility of celecoxib in a DGME/water mixture is significantly higher than in a PEG-400/water mixture at the same ratio of mixture ingredients. Without being bound by theory, it is believed that in the aqueous environment of the gastrointestinal tract, significantly more celecoxib will remain in solution, and hence available for immediate absorption, when delivered in a DGME-based solvent liquid than when the solvent liquid is based on PEG-400.

### Example 10

Soft gelatin encapsulated formulations F1, F3, F4, F5, F7, F8, F9 and F10 were prepared having components as shown in Table 12, below. Each formulation was hand-filled into soft gelatin capsules in a final amount of 0.9 g or 0.8 g, containing 200 mg of celecoxib, per capsule, and sealed.

TABLE 13
Composition (mg/capsule) of soft gelatin
capsule formulations

Formulation

| No. | F1 | F3 | F4 | F5 | F7 | F8 | F9 | F10 |
|---|---|---|---|---|---|---|---|---|
| Celecoxib | 200 | 200 | 200 | 200 | 200 | 200 | 200 | 200 |
| Labrasol ™ ¹ | 280 | — | 350 | — | — | — | — | 240 |
| DGME | 280 | 210 | 350 | 210 | 280 | 240 | 180 | 240 |
| Tagat ™ TO ² | — | 245 | — | 406 | 350 | 300 | 348 | — |
| Capmul ™ | — | 245 | — | 84 | 70 | 60 | 72 | — |
| MCM ³ | | | | | | | | |
| Propylene gly- | 140 | — | — | — | — | — | — | 120 |
| col laurate | | | | | | | | |
| Total | 900 | 900 | 900 | 900 | 900 | 800 | 800 | 800 |

¹ Labrasol ™ = polyoxyethylene (8) caprylic/capric glycerides
² Tagat ™ TO = polyoxyethylene glyceryl trioleate
³ Capmul ™ MCM = caprylic/capric mono- and diglycerides

### Example 11

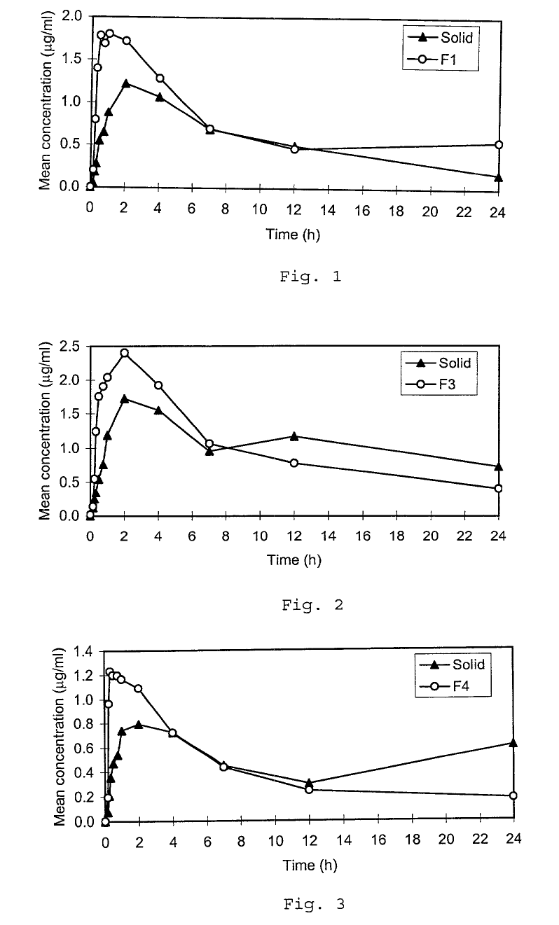

A study was performed in order to determine pharmacokinetic properties of celecoxib formulations F1, F3 and F4 of Example 8, in male beagle dogs. Twenty four dogs (Marshall Farms, North Pose, N.Y.) weighing approximately 7 to 9 kg and approximately 15 to 19 months of age were randomly divided into three groups and acclimated for 5 days. The general environment was maintained as follows: temperature 18.3° C.; humidity 40% or greater; approximately a 12-hour light, 12-hour dark cycle. The dogs were fasted overnight prior to dosing and for at least 4 hours post-dose. PMI Certified Canine Chow Diet # 5007 (PMI Nutrition Inc., Brentwood, Mo.) was available ad libitum to the animals throughout the study. Water from a reverse-osmosis water system was also available ad libitum. Each group received an oral dose of solid celecoxib in capsule form for comparison, followed by an oral dose of formulation F1, F3 or F4, in a two-way cross-over design. A five day washout period was provided between doses. Celecoxib was administered at a dose of 200 mg per animal and venous blood was collected pre-dose, and at 10, 15, 20, 30 and 45 minutes and 1, 2, 4, 7, 12 and 24 hours post-dose. Plasma was separated from blood by centrifugation at 3000× G and samples were stored at −20° C. until analysis. Concentrations of celecoxib in plasma were determined using an HPLC assay. Results are shown in FIGS. 1, 2 and 3.

In general, solvent liquid compositions containing diethylene glycol monoethyl ether and formulated in soft gelatin capsules exhibited superior rapid-onset pharmacokinetic profiles compared to solid capsule formulations. For example, overall, the soft gelatin capsules exhibited higher maximum plasma concentrations (Cₘₐₓ), and faster time to maximum plasma concentration (Tₘₐₓ)

### Example 10

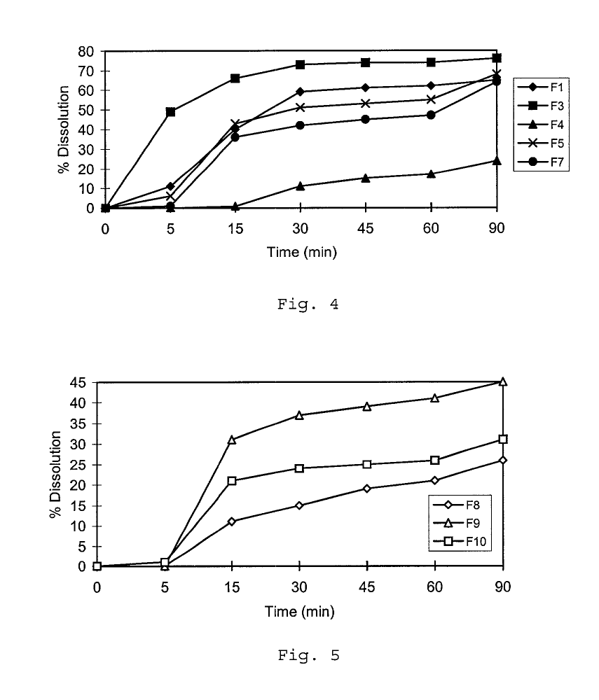

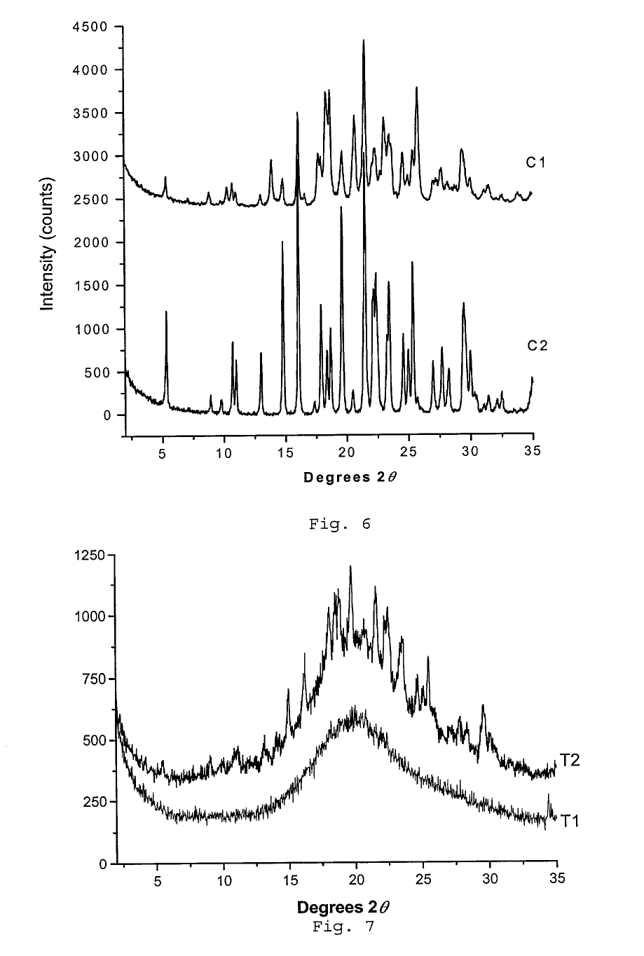

Celecoxib dissolution rates were measured in vitro for each of the soft gelatin capsule formulations described in Example 8, in a standard USP dissolution assay under the following conditions. USP apparatus II paddles were used to stir a dissolution medium (1 liter water containing 1% sodium dodecyl sulfate) at a speed of 75 rpm and a temperature of 37° C. After stirring for 90 minutes, an infinity time point was achieved by stirring at 250 rpm. The medium was then filtered through 10 mm Van-Kel filters. Samples were analyzed for celecoxib via UV detection. Dissolution rates for each of the formulations are shown in FIGS. 4 and 5.

It will be understood that in vitro dissolution rates obtained by the above procedure are not necessarily indicative in absolute terms of the process of release of celecoxib from an encapsulated solution in the gastrointestinal tract. However, it is believed that in relative terms a formulation exhibiting more rapid or complete dissolution in this assay will provide faster release in the gastrointestinal tract, and thereby faster onset of therapeutic effect.

It will be noted in FIG. 4 that among the 900 mg capsule formulations containing 200 mg celecoxib, the most rapid and complete in vitro dissolution was obtained with F3, wherein the solvent liquid comprises DGME accompanied by two co-solvents, polyoxyethylene glyceryl trioleate (Tagat™ TO) and caprylic/capric mono- and diglycerides (Capmul™ MCM).

### Example 11

A celecoxib drug substance C1 and celecoxib-polymer composites C3 and C4 were prepared by the following spray drying process. Celecoxib in crystalline form (a celecoxib drug substance C2 of prior art) was added to a solvent, with stirring at a temperature of 70-75° C., to prepare solutions S1, S3 and S4 having the composition shown in Table 14. Solutions S1 and S4 were prepared in 95% ethanol. Solution S3 was prepared in 70% isopropanol.

TABLE 14
Composition (mg/ml) of solutions S1, S3 and S4.

| Component | S1 | S3 | S4 |
|---|---|---|---|
| Celecoxib | 30 | 100 | 30 |
| HPMC | — | 50 | — |
| Povidone | — | — | 15 |

Each of solutions S1, S3 and S4 was spray dried individually at room temperature using a Yamato GB-21 spray dryer to form powders C1, C3 and C4, respectively, under the following conditions: (a) liquid flow rate of 10 ml/min; (b) inlet air temperature of 115° C.; (c) outlet air temperature of 75° C., and (d) drying airflow of 3.75 TMF. Powders C3 and C4 are celecoxib-polymer composites of the invention, each comprising 67% celecoxib and 33% polymer.

### Example 12

A celecoxib drug substance C10 was prepared by the following melt/quench cool process.

Approximately 5 g of crystalline celecoxib (the prior art celecoxib drug substance C2) was weighed into a metal foil tray and placed in an oven at 180° C. for 5 minutes to melt the celecoxib. This was then quench cooled by immersing the foil tray containing the melted celecoxib in liquid nitrogen, resulting in the formation of celecoxib drug substance C10 of the present invention. This drug substance could be gently ground by mortar and pestle to produce a celecoxib drug substance powder.

### Example 13

Powder X-ray diffraction (PXRD) analysis was used to determine the relative crystalline and amorphous celecoxib content of celecoxib drug substance C1 and celecoxib-polymer composites C3 and C4 as prepared in Example 11, by comparison with crystalline celecoxib drug substance C2. Data were collected using a Scintag Advanced Diffraction System operating under Scintag DMS/NT software. This system uses a peltier cooled solid state detector and a copper X-ray source maintained at 45 kV and 40 mA to provide CuKα₁ emission at 1.5406 Å. The beam aperture was controlled using tube divergence and anti-scatter slits of 2 and 4 mm respectively, while the detector anti-scatter and receiving slits were set at 0.5 and 0.3 mm respectively. Data were collected from 2° to 350 two-theta (2θ) using a scan step of 0.03°/point and a one second/point integration time. The samples were prepared using Scintag round top-loading stainless steel sample cups, and were fitted with 12 mm diameter aluminum inserts to accommodate small sample volumes.

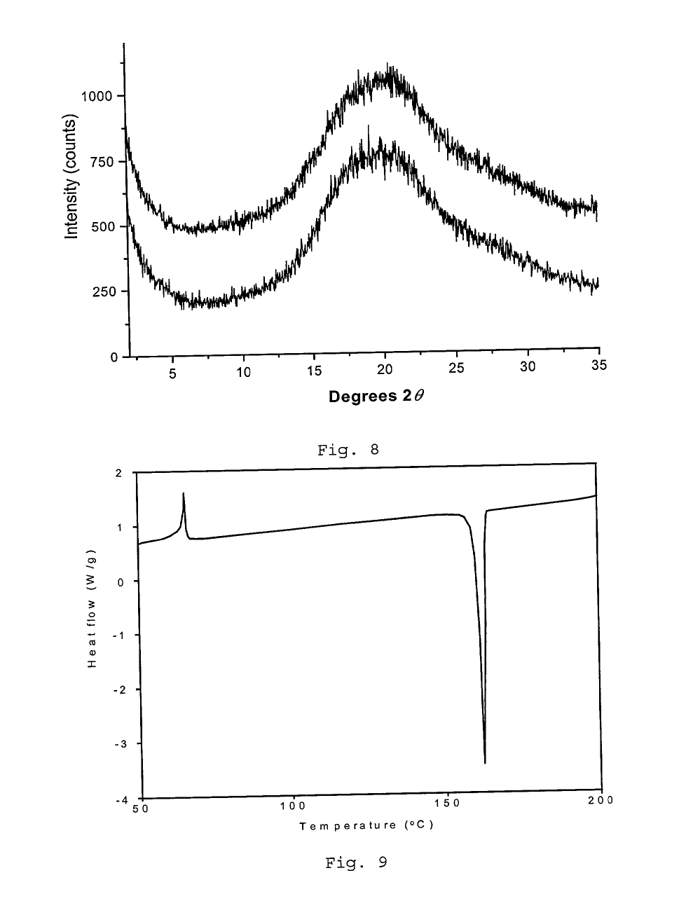

The results of the PXRD analyses are shown as bands in FIGS. 6-8. The appearance of larger, spiked peaks on a band indicates crystallinity whereas compressed peaks are indicative of amorphous material.

FIG. 6 shows that celecoxib alone (with no polymer) spray dried from an ethanol solution (C1) produced a strong crystalline signal similar to that of a crystalline celecoxib control (C2). If there is an amorphous component in celecoxib drug substance C1 it is a minor component.

FIG. 7 shows that when celecoxib was spray dried with HPMC (2:1 ratio by weight), the resulting celecoxib-polymer composite C3 was initially (at time T1) non-crystalline, i.e., the celecoxib in this composite was substantially phase pure amorphous celecoxib. When analysis was conducted on a sample that had been stored for two weeks at 40° C. and 75% relative humidity (at time T2), some recrystallization had occurred, as indicated by presence of crystalline peaks.

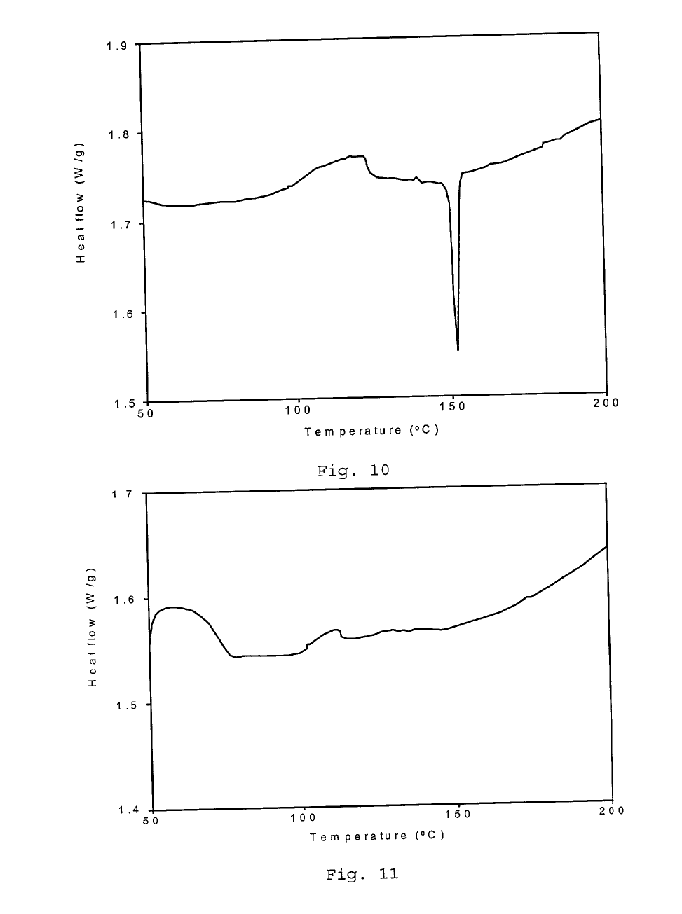

FIG. 8 shows that when celecoxib was spray dried with povidone (2:1 ratio by weight) the resulting celecoxib-polymer composite C4 was initially (at time T1) non-crystalline, i.e., the celecoxib in this composite was substantially phase pure amorphous celecoxib. When analysis was conducted on a sample that had been stored for two weeks at 40° C. and 75% relative humidity (at time T2), essentially no recrystallization had occurred, as indicated by absence of crystalline peaks.

### Example 14

Differential scanning calorimetry (DSC) was used to determine relative crystalline and amorphous celecoxib content of celecoxib drug substance C1 and celecoxib-polymer composites C3 and C4 as prepared in Example 13. DSC was performed using a TA Instruments DSC₂₉₂₀ differential scanning calorimeter with parameters set as follows: (a) temperature range of 50-200° C.; (b) heating rate of 2° C./min, modulating ±0.50C every 30 sec; (c) sample size of 3 mg; (d) hermetically sealed aluminum pans.

FIGS. 9-11 show DSC thermograms for the spray dried powders of Example 11.

FIG. 9 displays a thermogram for celecoxib drug product C1, exhibiting a large melting endotherm at 159.4° C. (onset) with an area of 96.42 J/g. No other transitions are evident. The magnitude of the endotherm suggests that a substantial portion of C1 was crystalline. Any amorphous celecoxib present in the sample was not detectable by this technique.

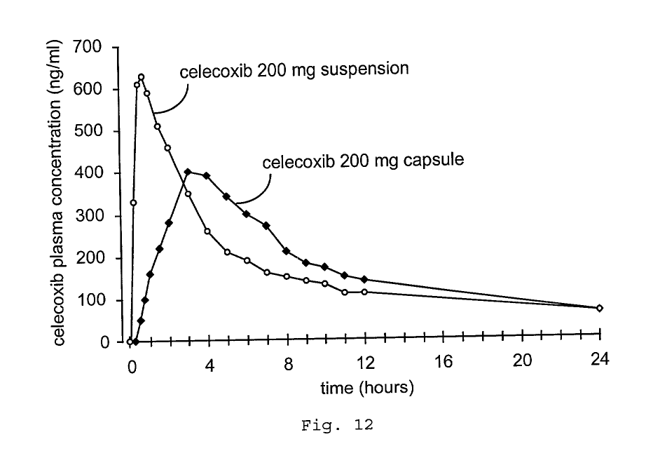

FIG. 10 displays a thermogram for celecoxib-polymer composite C3 (2:1 celecoxib:HPMC ratio). This material exhibits an apparent glass transition at 122.9° C. (onset), followed by a small melting endotherm at 150.1° C. with an area of 4.379 J/g. The endotherm indicates that most of the celecoxib in C3 is amorphous, but that a small amount of crystalline celecoxib is present.

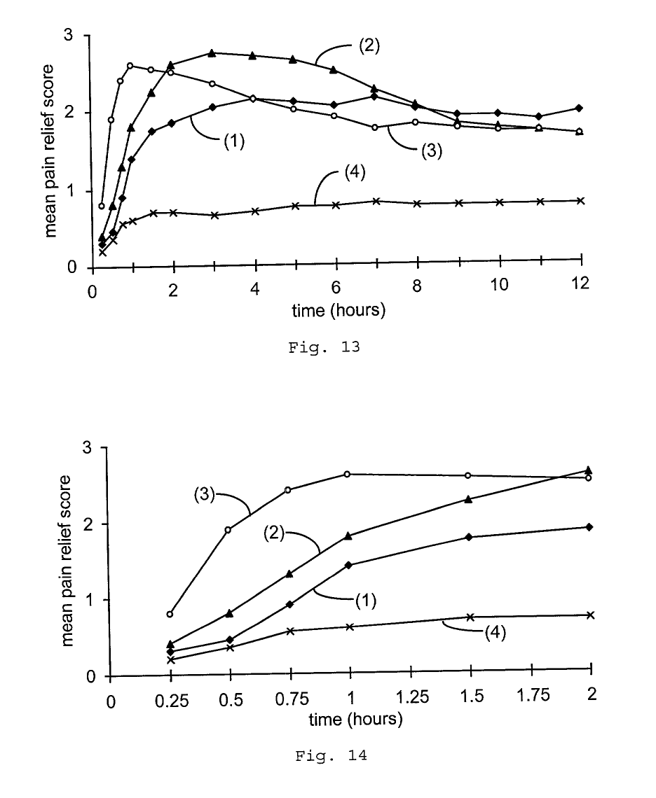

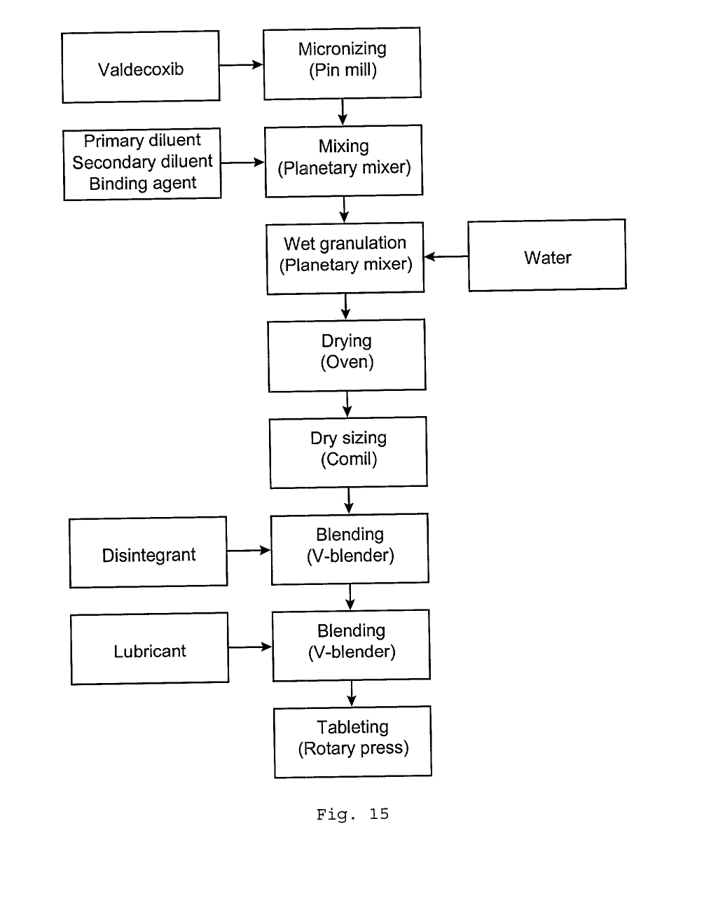

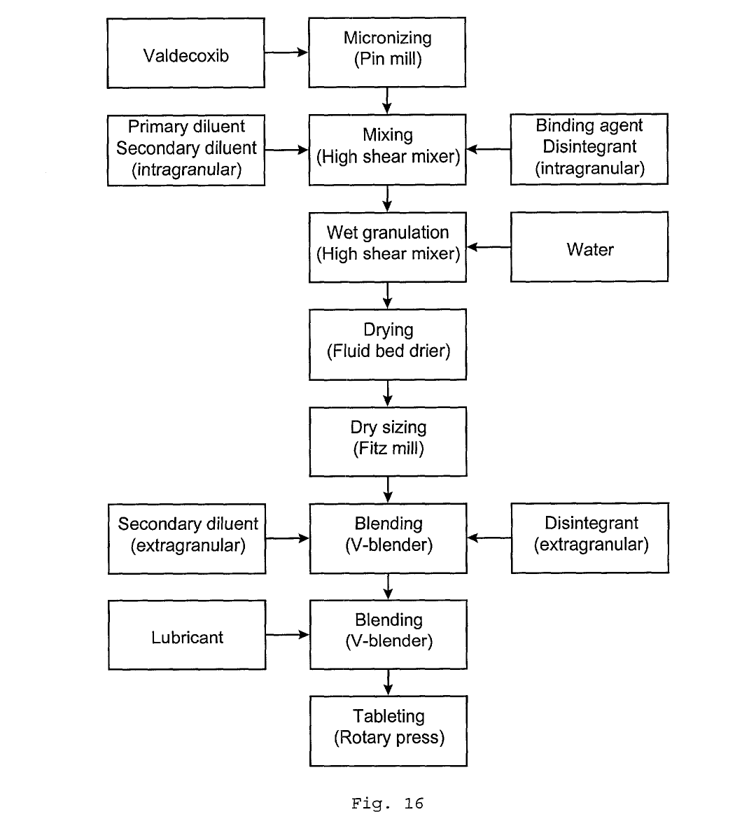

FIG. 11 displays a thermogram for celecoxib-polymer composite C4 (2:1 celecoxib:povidone ratio). This material exhibits an apparent glass transition at 111.4° C. (onset). No other transitions are evident, indicating that the material is substantially phase pure amorphous celecoxib.

### Example 15

DSC was also used to determine relative crystalline and amorphous content of celecoxib drug substance C10 prepared as in Example 12. DSC was performed using a TA Instruments MDSC differential scanning calorimeter at a scan rate of 5° C./min.

A first significant thermal event was observed at about 54° C., representing a glass transition temperature indicative of amorphous celecoxib. An exothermic peak observed at 100-105° C. was consistent with a crystallization event and represents conversion of amorphous celecoxib to a crystalline state. As was shown by the presence of a endothermic peak, the resulting crystalline celecoxib melted at about 165° C.

### Example 16

Tablets having the composition shown in Table 15 were prepared from celecoxib-polymer composite C4 by the following procedure. Composite C4, sodium lauryl sulfate and effervescent agents (citric acid and sodium bicarbonate) were admixed and milled for 10 min in a McCrone mill to form a powder mixture. The powder mixture was ground together with lactose, microcrystalline cellulose and sodium starch glycolate using a mortar and pestle to form a ground powder mixture. The ground powder mixture was then compressed using a Carver press to form tablets, which are illustrative of a pharmaceutical composition of the invention.

TABLE 15
Composition of tablets prepared from celecoxib-
polymer composite C4

| | Amount/tablet |
|---|---|
| Component | (mg) |
| Composite C4 | 300 |
| Sodium lauryl sulfate | 3 |
| Citric acid | 15.9 |
| Sodium bicarbonate | 25.2 |
| Lactose | 50 |
| Microcrystalline | 57 |
| cellulose | |
| Sodium starch | 48 |
| glycolate | |
| Total | 499 |

### Example 17

Tablets prepared as described in Example 16 were compared with a crystalline celecoxib capsule in an in vivo bioavailability assay in dogs. In a crossover design, each of six beagle dogs received a 200 mg dose of celecoxib in the form of the tablet composition of Example 16, and then after a washout period, the dogs each received a 200 mg dose of celecoxib in the form of a commercial Celebrex® 200 mg capsule, which contains celecoxib entirely in crystalline form. Blood plasma was collected pre-dose and at 0.25, 0.5, 0.75, 1, 1.5, 2, 3, 5, 8 and 24 hours post-dose. Celecoxib concentrations in plasma were measured using liquid chromatography/mass spectrometry. Cₘₐₓ, Tₘₐₓ and AUC (area under the curve, a measure of total bioavailability) were calculated from the data in accordance with standard procedure in the art. Mean results for all dogs are shown in Table 16.

The tablet of Example 16 prepared from amorphous celecoxib exhibited a significantly greater Cₘₐₓ (maximum blood plasma concentration), a comparable Tₘₐₓ, and a significantly greater AUC than the capsule formulated from crystalline celecoxib. As a measure of relative onset time, the time taken for the tablet of the invention to reach a plasma concentration equal to the Cₘₐₓ of the crystalline celecoxib capsule was only 0.5 hour, by comparison with 1.2 hours (the Tₘₐₓ for the crystalline celecoxib capsule).

TABLE 16
Bioavailability of the amorphous celecoxib
tablet of Example 16 by comparison with a
capsule of crystalline celecoxib

| | Tablet, | Capsule, |
|---|---|---|
| | amorphous | crystalline |
| Tₘₐₓ (h) | 1.4 | 1.2 |
| Cₘₐₓ (ng/ml) | 2130 | 1011 |
| AUC (ng/ml*h) | 17900 | 8470 |
| Relative onset | 0.5 | — |
| time (h) | | |

### Example 18

Tablets were prepared having the composition shown in Table 17.

TABLE 17

| Component | Function | Amount (mg) |
|---|---|---|
| valdecoxib, | active | 10 |
| micronized | ingredient | |
| lactose monohydrate | primary | 105 |
| NF, #310 | diluent | |
| microcrystalline | secondary | 60 |
| cellulose NF (Avicel ™ | diluent | |
| PH-101) | | |
| pregelatinized starch | binding | 20 |
| NF (National Starch | agent | |
| 1500) | | |
| croscarmellose sodium | disintegrant | 4 |
| NF (Ac-Di-Sol ™) | | |
| magnesium stearate | lubricant | 1 |
| Total tablet weight | | 200 |

The appropriate amount of micronized valdecoxib for the batch size was first mixed with an equal amount of lactose monohydrate, screened by passing through a 20 mesh screen, and added to a Hobart planetary mixer. The balance of the lactose monohydrate and the microcrystallized cellulose were then added to the mixer, which was then operated at a slow impeller speed for about 10 minutes. The resulting premix was then granulated in the planetary mixer by adding purified water manually over 12-15 minutes while continuing to mix at a slow to medium impeller speed. The resulting wet granules were dried on trays in a Gruenberg oven with an inlet air temperature of 60±5° C. to a moisture content of 2.0±1.0%, measured by loss on drying. The resulting dry granules were sized through a size 14 screen using a Quadro comil at medium speed, and then placed in a Patterson Kelley V-blender together with the croscarmellose sodium. The V-blender was operated for about 5 minutes to thoroughly mix the croscarmellose sodium with the granules; then magnesium stearate was added with further mixing for about 3 minutes to prepare a lubricated blend. This was compressed on a Manesty DB16 rotary press using 7.5 mm standard concave tooling to provide a tablet weight of 200±10 mg having a hardness of 10±4 kP.

### Example 19

Tablets were prepared having the composition shown in Table 18.

TABLE 18

| Component | Function | Amount (mg) |
|---|---|---|
| valdecoxib, | active | 10 |
| micronized | ingredient | |
| lactose monohydrate | primary | 103 |
| NF, #310 | diluent | |
| microcrystalline | secondary | 60 |
| cellulose NF (Avicel ™ | diluent | |
| PH-101) | | |
| intragranular | | 30 |
| extragranular | | 30 |
| pregelatinized starch | binding | 20 |
| NF (National Starch | agent | |
| 1500) | | |
| croscarmellose sodium | disintegrant | 6 |
| NF (Ac-Di-Sol ™) | | |
| intragranular | | 3 |
| extragranular | | 3 |
| magnesium stearate | lubricant | 1 |
| Total tablet weight | | 200 |

The micronized valdecoxib, lactose monohydrate, intragranular microcrystalline cellulose, pregelatinized starch and intragranular croscarmellose sodium were mixed in a Baker Perkins high shear mixer at high impeller/chopper speed for about 3 minutes to form a premix. Purified water was added to the premix via a Watson Marlow peristaltic pump over a period of about 3 minutes and mixing continued for a further 45 seconds. The resulting wet granules were dried in an Aeromatic fluid bed drier with an inlet air temperature of 60±5° C. to a moisture content of 2.0±1.0% as measured by loss on drying, to form a dry granulate. The dry granulate was sized through a 20 mesh screen using a Fitz mill with knives forward, at 1800 rpm, and was then placed in a Patterson Kelley V-blender. Here, the granulate was mixed with the extragranular microcrystalline cellulose and extragranular croscarmellose sodium for about 5 minutes, and then with the magnesium stearate for a further 3 minutes, to form a lubricated blend. This was compressed on a Korsch PH-230 rotary press using 7.5 mm standard concave tooling to provide a tablet weight of 200±10 mg. Tablets were prepared having hardnesses of 6, 8, 10 and 12 kP.

### Example 20

Using the process of Example 19, tablets were prepared having the composition shown in Table 19. Tablets were film coated with Opadry Yellow YS-1-12525A or Opadry White YS-1-18027A at 3% of uncoated tablet weight, using a 15% suspension of the coating material in water.

TABLE 19

| Ingredient | Amount/tablet (mg) | | | |
|---|---|---|---|---|
| Valdecoxib, micronized | 5 | 10 | 20 | 40 |
| Lactose monohydrate NF | 108 | 103 | 206 | 186 |
| Microcrystalline cellulose NF | 60 | 60 | 120 | 120 |
| Pregelatinized starch NF | 20 | 20 | 40 | 40 |
| Croscarmellose sodium NF | 6 | 6 | 12 | 12 |
| Magnesium stearate NF | 1 | 1 | 2 | 2 |
| Total weight (excluding coating) | 200 | 200 | 400 | 400 |
| Opadry Yellow YS-1-12525A | 6 | | | 12 |
| Opadry White YS-1-18027A | | 6 | 12 | |

Properties of the tablets of Example 20 are presented in Table 20.

Disintegration was evaluated by the following procedure. Six identical tablets were separately placed into one of six tubes having a wire mesh screen bottom in a disintegration basket. A water bath was preheated to 37° C.±2° C. and maintained at that temperature for the duration of the disintegration test. A 1000 ml beaker was placed in the water bath. The beaker was filled with a sufficient amount of water to ensure that the wire mesh screen of the tubes would remain at least 2.5 cm below the water surface during the test. The disintegration basket was inserted in the water and repeatedly raised and lowered until the test was complete while maintaining the wire mesh screen of the tubes at least 2.5 cm below the water surface. Disintegration time for each tablet was the time, measured from time of insertion of the basket, at which the very last portion of the tablet passed through the screen at the bottom of the tube.

TABLE 20

| | 5 mg | 10 mg | 20 mg | 40 mg |
|---|---|---|---|---|
| Shape | oval | caplet | caplet | heptagon |
| Thickness (mm) | 3.6 ± 0.2 | 3.6 ± 0.2 | 4.8 ± 0.4 | 4.2 ± 0.3 |
| Hardness (kP) | 9 ± 3 | 9 ± 3 | 13 ± 5 | 13 ± 5 |
| Friability (%) | <0.8 | <0.8 | <0.8 | <0.8 |
| Disintegration | 12 | 12 | 12 | 12 |
| in vitro | minutes | minutes | minutes | minutes |

### Example 21

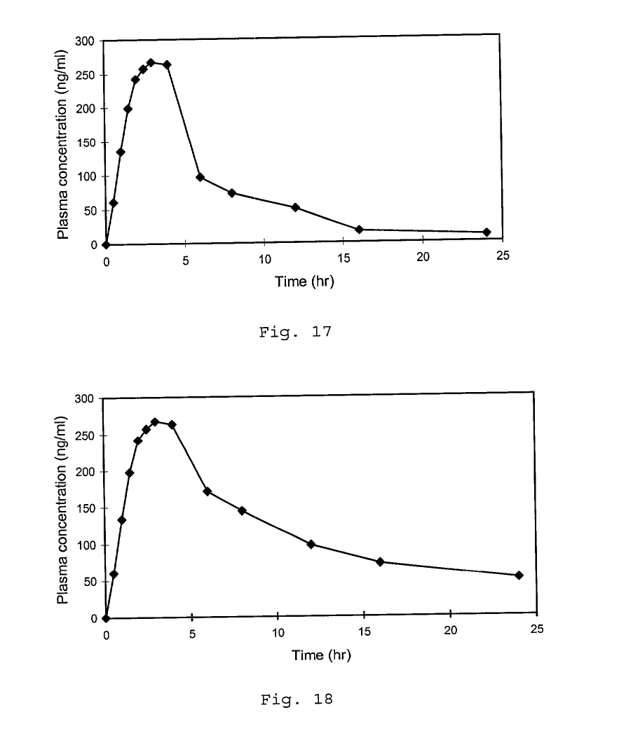

A study was performed in order to determine pharmacokinetic properties of the valdecoxib composition of Example 19, in 23 beagle dogs. Valdecoxib was administered at a dose of 20 mg (2 tablets). Venous blood was collected pre-dose, and at 0.5, 1, 1.5, 2, 2.5, 3, 4, 6, 8, 12 and 24 hours after oral dose administration. Plasma was separated from blood by centrifugation at 3000 G and samples were stored at −20° C. until analysis. Concentrations of valdecoxib in plasma were determined using an HPLC assay. Results are shown in FIG. 17.

### Example 22

A study was performed in order to determine pharmacokinetic properties of the valdecoxib composition of Example 19, in 24 healthy adult humans. Valdecoxib was administered at a dose of 20 mg (2 tablets). Venous blood was collected pre-dose, and at 0.5, 1, 1.5, 2, 2.5, 3, 4, 6, 8, 12, 16 and 24 hours after oral dose administration. Plasma was separated from blood by centrifugation at 3000 G and samples were stored at −20° C. until analysis. Concentrations of valdecoxib in plasma were determined using an HPLC assay. Results are shown in FIG. 18.

Calculated Cₘₐₓ was 303±93 ng/ml. Calculated Tₘₐₓ was 2.97±0.73 h.

### Example 23

The combination of the invention of a cyclooxygenase-2 inhibitor and a vasomodulator, preferably caffeine, could be formulated in any of the above formulations or delivery vehicles. The cyclooxygenase-2 inhibitor could be administered in a single dose with the vasomodulator or sequentially or concurrently. Addition of a vasomodulator to the cyclooxygenase-2 inhibitor substance is expected to exhibit similar pharmacokinetic profiles as seen in the examples of cyclooxygenase-2 inhibitor drug substances hereinabove.

Preferably, a therapeutic combination administered combined in a single dosage form is a single tablet, pill or capsule of said single dosage form comprising a selective cyclooxygenase-2 inhibitor in an amount of from about 0.1 mg to about 2000 mg, and caffeine in an amount of about 1 to 500 mg. More preferably, a single tablet, pill or capsule of said single dosage form comprises a selective cyclooxygenase-2 inhibitor is in an amount of from about 0.5 mg to about 500 mg, and caffeine in an amount of about 10 to 400 mg. Still more preferably, the dosage form comprises a selective cyclooxygenase-2 inhibitor in an amount of from about 1 mg to about 200 mg, and caffeine in an amount of about 20 to 300 mg. Still more preferably, the dosage form comprises a selective cyclooxygenase-2 inhibitor in an amount of from about 1 mg to about 200 mg, and caffeine in an amount of about 30 to 200 mg. Yet more preferably, the dosage form comprises a selective cyclooxygenase-2 inhibitor in an amount of from about 1 mg to about 200 mg, and caffeine in an amount of about 40 to 150 mg. More preferably, the dosage form comprises a selective cyclooxygenase-2 inhibitor in an amount of from about 1 mg to about 200 mg, and caffeine in an amount of about 55 to 100 mg.

What is claimed is:

1. A therapeutic combination useful in the treatment, amelioration, prevention, or delay of pain comprising a high energy form of a selective cyclooxygenase-2 inhibitor, a vasomodulator, and a pharmaceutically acceptable excipient, carrier, or diluent, the cyclooxygenase-2 inhibitor and vasomodulator each being present in an amount effective to contribute to the treatment, prevention, ameloriation or delay of pain.

2. The combination of claim 1 wherein the vasomodulator is a vasoconstrictor.

3. The combination of claim 1 wherein the vasomodulator is a vasodilator.

4. The combination of claim 1 wherein the vasomodulator is a xanthine compound or salt or derivative thereof.

5. The combination of claim 4 wherein the xanthine compound is selected from the group consisting of caffeine, theobromine, theophylline, and xanthine.

6. The combination of claim 5 wherein the xanthine compound is caffeine.

7. The combination of claim 5 wherein the xanthine compound is theobromine.

8. The combination of claim 5 wherein the xanthine compound is theophylline.

9. The combination according to any of claims 1-4 wherein the pain is generalized pain.

10. The combination according to any of claims 1-4 wherein the pain is headache pain.

11. The combination according claim 10 wherein the headache pain is selected from the group consisting of migraine headache pain, cluster headache pain, chronic headache pain, substance-induced headache pain, tension or stress related headache pain, sinus headache pain, pain resulting from anesthesia, headache pain associated with increased intracranial pressure, headache pain associated with decreased intracranial pressure, headache pain resulting from giant cell arteritis, and headache pain resulting from lumbar puncture.

12. The combination of claim 5 wherein the selective cyclooxygenase-2 inhibitor is selected from compounds of Formula II: 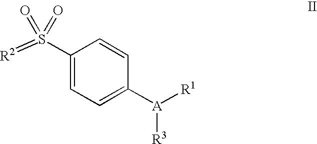
wherein A is selected from the group consisting of partially unsaturated or unsaturated heterocyclyl and partially unsaturated or unsaturated carbocyclic rings;
wherein R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
wherein R² is selected from the group consisting of methyl or amino; and
wherein R³ is selected from the group consisting of a radical selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt thereof.

13. The combination of claim 4 wherein the cyclooxygenase inhibitor is selected from compounds, and their pharmaceutically acceptable salts, of the group consisting of:
5-(4-fluorophenyl)-1-[4-(methylsulfonyl)phenyl]-3-(trifluoromethyl)pyrazole;
4-(4-fluorophenyl)-5-[4-(methylsulfonyl)phenyl]-1-phenyl-3-(trifluoromethyl)pyrazole;
4-(5-(4-chlorophenyl)-3-(4-methoxyphenyl)-1H-pyrazol-1-yl)benzenesulfonamide
4-(3,5-bis(4-methylphenyl)-1H-pyrazol-1-yl)benzenesulfonamide;
4-(5-(4-chlorophenyl)-3-phenyl-1H-pyrazol-1-yl)benzenesulfonamide;
4-(3,5-bis(4-methoxyphenyl)-1H-pyrazol-1-yl)benzenesulfonamide;
4-(5-(4-chlorophenyl)-3-(4-methylphenyl)-1H-pyrazol-1-yl)benzenesulfonamide;
4-(5-(4-chlorophenyl)-3-(4-nitrophenyl)-1H-pyrazol-1-yl)benzenesulfonamide;
4-(5-(4-chlorophenyl)-3-(5-chloro-2-thienyl)-1H-pyrazol-1-yl)benzenesulfonamide;
4-(4-chloro-3,5-diphenyl-1H-pyrazol-1-yl)benzenesulfonamide
4-[5-(4-chlorophenyl)-3-(trifluoromethyl)-1H-pyrazol-1-yl]benzenesulfonamide;
4-[5-phenyl-3-(trifluoromethyl)-1H-pyrazol-1-yl]benzenesulfonamide;
4-[5-(4-fluorophenyl)-3-(trifluoromethyl)-1H-pyrazol-1-yl]benzenesulfonamide;
4-[5-(4-methoxyphenyl)-3-(trifluoromethyl)-1H-pyrazol-1-yl]benzenesulfonamide;
4-[5-(4-chlorophenyl)-3-(difluoromethyl)-1H-pyrazol-1-yl]benzenesulfonamide;
4-[5-(4-methylphenyl)-3-(trifluoromethyl)-1H-pyrazol-1-yl]benzenesulfonamide;
4-[4-chloro-5-(4-chlorophenyl)-3-(trifluoromethyl)-1H-pyrazol-1-yl]benzenesulfonamide;
4-[3-(difluoromethyl)-5-(4-methylphenyl)-1H-pyrazol-1-yl]benzenesulfonamide;
4-[3-(difluoromethyl)-5-phenyl-1H-pyrazol-1-yl]benzenesulfonamide;
4-[3-(difluoromethyl)-5-(4-methoxyphenyl)-1H-pyrazol-1-yl]benzenesulfonamide;
4-[3-cyano-5-(4-fluorophenyl)-1H-pyrazol-1-yl]benzenesulfonamide; 4-[3-(difluoromethyl)-5-(3-fluoro-4-methoxyphenyl)-1H-pyrazol-1-yl]benzenesulfonamide;
4-[5-(3-fluoro-4-methoxyphenyl)-3-(trifluoromethyl)-1H-pyrazol-1-yl]benzenesulfonamide;
4-[4-chloro-5-phenyl-1H-pyrazol-1-yl]benzenesulfonamide;
4-[5-(4-chlorophenyl)-3-(hydroxymethyl)-1H-pyrazol-1-yl]benzenesulfonamide;
4-[5-(4-(N,N-dimethylamino)phenyl)-3-(trifluoromethyl)-1H-pyrazol-1-yl]benzenesulfonamide;
4-[4-(4-fluorophenyl)-1,1-dimethylcyclopenta-2,4-dien-3-yl]benzenesulfonamide;
4-[2-(4-methylpyridin-2-yl)-4-(trifluoromethyl)-1H-imidazol-1-yl]benzenesulfonamide; 4-[2-(5-methylpyridin-3-yl)-4-(trifluoromethyl)-1H-imidazol-1-yl]benzenesulfonamide;
4-[2-(2-methylpyridin-3-yl)-4-(trifluoromethyl)-1H-imidazol-1-yl]benzenesulfonamide;
3-[1-[4-(methylsulfonyl)phenyl]-4-(trifluoromethyl)-1H-imidazol-2-yl]pyridine;
2-[1-[4-(methylsulfonyl)phenyl-4-(trifluoromethyl)-1H-imidazol-2-yl]pyridine;
4-[2-(3-chloro-4-methylphenyl)-4-(trifluoromethyl)-1H-imidazol-1-yl]benzenesulfonamide;
4-[2-(3-fluoro-5-methylphenyl)-4-(trifluoromethyl)-1H-imidazol-1-yl]benzenesulfonamide;
4-[2-(3-methylphenyl)-4-trifluoromethyl-1H-imidazol-1-yl]benzenesulfonamide;
4-[2-(4-methoxy-3-chlorophenyl)-4-trifluoromethyl-1H-imidazol-1-yl]benzenesulfonamide;
1-allyl-4-(4-fluorophenyl)-3-[4-(methylsulfonyl)phenyl]-5-(trifluoromethyl)-1H-pyrazole;
4-[1-ethyl-4-(4-fluorophenyl)-5-(trifluoromethyl)-1H-pyrazol-3-yl]benzenesulfonamide;
N-phenyl-[4-(4-luorophenyl)-3-[4-(methylsulfonyl)phenyl]-5-(trifluoromethyl)-1H-pyrazol-1-yl]acetamide; ethyl [4-(4-fluorophenyl)-3-[4-(methylsulfonyl)phenyl]-5-(trifluoromethyl)-1H-pyrazol-1-yl]acetate;
4-(4-fluorophenyl)-3-[4-(methylsulfonyl)phenyl]-1-(2-phenylethyl)-1H-pyrazole;
4-(4-fluorophenyl)-3-[4-(methylsulfonyl)phenyl]-1-(2-phenylethyl)-5-(trifluoromethyl)pyrazole;
1-ethyl-4-(4-fluorophenyl)-3-[4-(methylsulfonyl)phenyl]-5-(trifluoromethyl)-1H-pyrazole;
5-(4-fluorophenyl)-2-methoxy-4-[4-(methylsulfonyl)phenyl]-6-(trifluoromethyl)pyridine;
2-ethoxy-5-(4-fluorophenyl)-4-[4-(methylsulfonyl)phenyl]-6-(trifluoromethyl)pyridine;
5-(4-fluorophenyl)-4-[4-(methylsulfonyl)phenyl]-2-(2-propynyloxy)-6-(trifluoromethyl)pyridine;
2-bromo-5-(4-fluorophenyl)-4-[4-(methylsulfonyl)phenyl]-6-(trifluoromethyl)pyridine;
4-[2-(3-chloro-4-methoxyphenyl)-4,5-difluorophenyl]benzenesulfonamide;
1-(4-fluorophenyl)-2-[4-(methylsulfonyl)phenyl]benzene;
5-difluoromethyl-4-(4-methylsulfonylphenyl)-3-phenylisoxazole;
4-[3-ethyl-5-phenylisoxazol-4-yl]benzenesulfonamide;
4-[5-difluoromethyl-3-phenylisoxazol-4-yl]benzenesulfonamide;
4-[5-hydroxymethyl-3-phenylisoxazol-4-yl] benzenesulfonamide;
4-[5-methyl-3-phenyl-isoxazol-4-yl]benzenesulfonamide;
ethyl 2-[4-(4-fluorophenyl)-5-[4-(methylsulfonyl) phenyl]oxazol-2-yl]-2-benzyl-acetate;
2-[4-(4-fluorophenyl)-5-[4-(methylsulfonyl)phenyl]oxazol-2-yl]acetic acid;
2-(tert-butyl)-4-(4-fluorophenyl)-5-[4-(methylsulfonyl)phenyl]oxazole;
4-(4-fluorophenyl)-5-[4-(methylsulfonyl)phenyl]-2-phenyloxazole;
4-(4-fluorophenyl)-2-methyl-S-[4-(methylsulfonyl)phenyl]oxazole;
4-[5-(3-fluoro-4-methoxyphenyl)-2-trifluoromethyl-4-oxazolyl]benzenesulfonamide;
4-[4-(methyl)-sulfonyl)phenyl]-3-phenyl-2 (5H)-furanone;
4-(5-methyl-3-phenyl-4-isoxazolyl); and
2-(6-methylpyrid-3-yl)-3-(4-methylsulfinylphenyl)-5-chloropyridine.

14. The combination of any of claims 1-4, wherein the selective cyclooxygenase-2 inhibitor is selected from the group of compounds of Formula I: 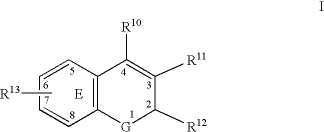
wherein G is selected from the group consisting of O or S or NR^{a}; wherein R^{a} is alkyl;
wherein R¹⁰ is selected from the group consisting of H and aryl
wherein R¹¹ is selected from the group consisting of carboxyl, aminocarbonyl, alkylsulfonylaminocarbonyl and alkoxycarbonyl;
wherein R¹² is selected from the group consisting of haloalkyl, alkyl, aralkyl, cycloalkyl and aryl optionally substituted with one or more radicals selected from alkylthio, nitro and alkylsulfonyl; and
wherein R¹³ is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
or wherein R¹³ together with ring E forms a naphthyl radical;
or a pharmaceutically acceptable salt or isomer or prodrug thereof.

15. The combination of claim 4 wherein the cyclooxygenase inhibitor is selected from compounds, and their pharmaceutically acceptable salts, of the group consisting of:
6-chloro-2-trifluoromethyl-2H-1-benzopyran-3-carboxylic acid;
6-chloro-7-methyl-2-trifluoromethyl-2H-1-benzopyran-3-carboxylic acid;
8-(1-methylethyl)-2-trifluoromethyl-2H-1-benzopyran-3-carboxylic acid;
6-chloro-7-(1,1-dimethylethyl)-2-trifluoromethyl-2H-1-benzopyran-3-carboxylic acid;
6-chloro-8-(1-methylethyl)-2-trifluoromethyl-2H-1-benzopyran-3-carboxylic acid;
2-trifluoromethyl-3H-naphthopyran-3-carboxylic acid;
7-(1,1-dimethylethyl)-2-trifluoromethyl-2H-1-benzopyran-3-carboxylic acid;
6-bromo-2-trifluoromethyl-2H-1-benzopyran-3-carboxylic acid;
8-chloro-2-trifluoromethyl-2H-1-benzopyran-3-carboxylic acid;
6-trifluoromethoxy-2-trifluoromethyl-2H-1-benzopyran-3-carboxylic acid;
5,7-dichloro-2-trifluoromethyl-2H-1-benzopyran-3-carboxylic acid;
8-phenyl-2-trifluoromethyl-2H-1-benzopyran-3-carboxylic acid;
7,8-dimethyl-2-trifluoromethyl-2H-1-benzopyran-3-carboxylic acid;
6,8-bis(dimethylethyl)-2-trifluoromethyl-2H-1-benzopyran-3-carboxylic acid;
7-(1-methylethyl)-2-trifluoromethyl-2H-1-benzopyran-3-carboxylic acid;
7-phenyl-2-trifluoromethyl-2H-1-benzopyran-3-carboxylic acid;
6-chloro-7-ethyl-2-trifluoromethyl-2H-1-benzopyran-3-carboxylic acid;
6-chloro-8-ethyl-2-trifluoromethyl-2H-1-benzopyran-3-carboxylic acid;
6-chloro-7-phenyl-2-trifluoromethyl-2H-1-benzopyran-3-carboxylic acid;
6,7-dichloro-2-trifluoromethyl-2H-1-benzopyran-3-carboxylic acid;
6,8-dichloro-2-trifluoromethyl-2H-1-benzopyran-3-carboxylic acid;
2-trifluoromethyl-3H-naptho[2,1-b]pyran-3-carboxylic acid;
6-chloro-8-methyl-2-trifluoromethyl-2H-1-benzopyran-3-carboxylic acid;
8-chloro-6-methyl-2-trifluoromethyl-2H-1-benzopyran-3-carboxylic acid;
8-chloro-6-methoxy-2-trifluoromethyl-2H-1-benzopyran-3-carboxylic acid;
6-bromo-8-chloro-2-trifluoromethyl-2H-1-benzopyran-3-carboxylic acid;
8-bromo-6-fluoro-2-trifluoromethyl-2H-1-benzopyran-3-carboxylic acid;
8-bromo-6-methyl-2-trifluoromethyl-2H-1-benzopyran-3-carboxylic acid;
8-bromo-5-fluoro-2-trifluoromethyl-2H-1-benzopyran-3-carboxylic acid;
6-chloro-8-fluoro-2-trifluoromethyl-2H-1-benzopyran-3-carboxylic acid;
6-bromo-8-methoxy-2-trifluoromethyl-2H-1-benzopyran-3-carboxylic acid;
6-[[(phenylmethyl)amino]sulfonyl]-2-trifluoromethyl-2H-1-benzopyran-3-carboxylic acid;
6-[(dimethylamino)sulfonyl]-2-trifluoromethyl-2H-1-benzopyran-3-carboxylic acid;
6-[(methylamino)sulfonyl]-2-trifluoromethyl-2H-1-benzopyran-3-carboxylic acid;
6-[(4-morpholino)sulfonyl]-2-trifluoromethyl-2H-1-benzopyran-3-carboxylic acid;
6-[(1,1-dimethylethyl)aminosulfonyl]-2-trifluoromethyl-2H-1-benzopyran-3-carboxylic acid;
6-[(2-methylpropyl)aminosulfonyl]-2-trifluoromethyl-2H-1-benzopyran-3-carboxylic acid;
6-methylsulfonyl-2-trifluoromethyl-2H-1-benzopyran-3-carboxylic acid;
8-chloro-6-[[(phenylmethyl)amino]sulfonyl]-2-trifluoromethyl-2H-1-benzopyran-3-carboxylic acid;
6-phenylacetyl-2-trifluoromethyl-2H-1-benzopyran-3-carboxylic acid;
6,8-dibromo-2-trifluoromethyl-2H-1-benzopyran-3-carboxylic acid;
8-chloro-5,6-dimethyl-2-trifluoromethyl-2H-1-benzopyran-3-carboxylic acid;
6,8-dichloro-(S)-2-trifluoromethyl-2H-1-benzopyran-3-carboxylic acid;
6-benzylsulfonyl-2-trifluoromethyl-2H-1-benzopyran-3-carboxylic acid;
6-[[N-(2-furylmethyl)amino]sulfonyl]-2-trifluoromethyl-2H-1-benzopyran-3-carboxylic acid;
6-[[N-(2-phenylethyl)amino]sulfonyl]-2-trifluoromethyl-2H-1-benzopyran-3-carboxylic acid;
6-iodo-2-trifluoromethyl-2H-1-benzopyran-3-carboxylic acid;
7-(1,1-dimethylethyl)-2-pentafluoroethyl-2H-1-benzopyran-3-carboxylic acid; and
6-chloro-2-trifluoromethyl-2H-1-benzothiopyran-3-carboxylic acid.

16. The combination of claim 6 wherein the selective cyclooxygenase-2 inhibitor is a compound selected from the group consisting of celecoxib, rofecoxib, valdecoxib, deracoxib, etoricoxib, 2-(3,4-Difluorophenyl)-4-(3-hydroxy-3-methylbutoxy)-5-[4-(methylsulfonyl)phenyl]-3(2H)-pyridazinone, parecoxib, and meloxicam.

17. The combination of any of claims 1-4, wherein the selective cyclooxygenase-2 inhibitor is selected from the group of compounds of Formula III: 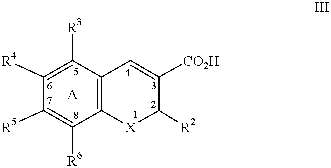
wherein X is O or S;
R² is lower haloalkyl;
R³is selected from the group consisting of hydrido and halo;
R⁴ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, a 5-membered nitrogen containing heterocyclosulfonyl, and a 6-membered nitrogen containing heterocyclosulfonyl;
R⁵ is selected from the group consisting of hydrido, lower alkyl, halo, lower alkoxy, and aryl; and
R⁶is selected from the group consisting of hydrido, halo, lower alkyl, lower alkoxy, and aryl. or a pharmaceutically acceptable salt, or isomer, or prodrug thereof.

18. The combination of any of claims 1-4, wherein the selective cyclooxygenase-2 inhibitor is selected from the group of compounds of Formula IV: 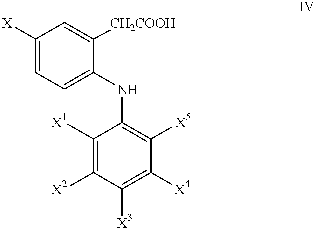
wherein X is methyl or ethyl;
X¹ is chloro or fluoro;
X² is hydrido or fluoro;
X³ is hydrido, fluoro, chloro, methyl, ethyl, methoxy, ethoxy, or hydroxy;
X⁴ is hydrido or fluoro; and
X⁵ is chloro, fluoro, trifluoromethyl or methyl;
pharmaceutically acceptable salts thereof; and
pharmaceutically acceptable prodrug esters thereof.

19. The combination of claim 1 wherein the selective cyclooxygenase-2 inhibitor and the vasomodulator are administered combined in a single dosage form.

20. The combination of claim 6 wherein the selective cyclooxygenase-2 inhibitor and the vasoconstrictor are administered combined in a single dosage form.

21. The combination of claim 6 wherein a single tablet, pill or capsule of the single dosage form comprises a selective cyclooxygenase-2 inhibitor in an amount of from about 0.1 mg to about 2000 mg, and caffeine in an amount of about 1 to 500 mg.

22. The combination of claim 6 wherein a single tablet, pill or capsule of the single dosage form comprises a selective cyclooxygenase-2 inhibitor in an amount of from about 1 mg to about 200 mg, and caffeine in an amount of about 55 to 100 mg.

23. The combination of claim 1 wherein the effective amount of a selective cyclooxygenase-2 inhibitor compound and an amount of caffeine are administered as separate dosage forms sequentially or concurrently.

24. The combination of claim 6 wherein the effective amount of a selective cyclooxygenase-2 inhibitor compound and an amount of caffeine are administered as separate dosage forms sequentially or concurrently.

25. The combination as set forth in claim 1 comprising an amorphous particulate selective cyclooxygenase-2 inhibitor.

26. The combination as set forth in claim 25 wherein the pain is headache pain.

27. The combination of claim 1 comprising a polar solvent in which the selective cyclyoxygenase-2 inhibitor is dissolved.

28. The combination of claim 27 comprising a co-solvent.

29. The combination of claim 28 wherein the co-solvent is substantially fully miscible with the solvent.

30. The combination of claim 27 wherein the pain is headache pain.

31. The combination of claim 27 wherein the polar solvent is a pharmaceutically acceptable glycol or glycol ether.

32. The combination of claim 27 further comprising a liquid vehicle.

33. The combination of claim 1 wherein the high energy form of the selective cyclooxygenase-2 inhibitor is a high energy solid state form.

34. The combination of claim 33 comprising an amorphous particulate selective cyclooxygenase-2 inhibitor.

35. The combination of claim 34 wherein the amorphous particulate selective cyclooxygenase-2 inhibitor is present in an amount of about 10% to about 100% by weight cyclooxygenase-2 inhibitor.

36. The combination of claim 34 wherein the amorphous particulate selective cyclooxygenase-2 inhibitor is amorphous celecoxib.

37. The combination of claim 34 wherein the amorphous celecoxib is a celecoxib-crystallization inhibitor composite comprising particles of amorphous celecoxib in intimate association with one or more crystallization inhibitor(s) in an amount effective to reduce transformation of amorphous celecoxib to crystalline celecoxib.

38. The combination of claim 1 wherein the formulation has one or more orally deliverable dose units, each comprising a combination of a selective cyclooxygenase-2 inhibitory compound and a vasomodulator in a therapeutically effective amount, wherein the compound is present in solid particles having a D₉₀ particle size of about 0.01 to about 200 μm, a sufficient portion by weight of the particles being smaller than 1 μm to provide a substantially higher Cₘₐₓ and/or a substantially shorter Tₘₐₓ by comparison with an otherwise similar composition wherein substantially all of the particles are larger than 1μm.

39. The combination of claim 38 wherein the compound is selected from the group consisting of celecoxib, deracoxib, valdecoxib and rofecoxib.

40. The combination of claim 1 wherein the cyclooxygenase-2 inhibitor is particulate valdecoxib and present in an amount of about 1 mg to about 100 mg per dose, with a therapeutically effective amount of the vasomodulator, and one or more pharmaceutically acceptable excipients, wherein a single dose, upon oral administration to a fasting subject, provides a time course of blood serum concentration of valdecoxib having at least one of
(a) a time to reach a threshold concentration for therapeutic effect not greater than about 0.5 h after administration;
(b) a time to reach maximum concentration (Tₘₐₓ) not greater than about 5 h after administration; and
(c) a maximum concentration (Cₘₐₓ) not less than about 100 ng/ml.

41. The combination of claim 40 wherein the threshold concentration for therapeutic effect is about 20 ng/ml.

42. The combination of claim 41 wherein a single does, upon oral administration to a fasting subject, provides a time course blood serum concentration of valdecoxib having each of a time to reach a concentration of 20 ng/ml not greater than about 0.5 h after administration;
a time to reach maximum concentration (Tₘₐₓ) not greater than about 3 h after administration; and
a maximum concentration (Cₘₐₓ) not less than about 100 ng/ml.

43. The combination of claim 40 wherein the valdecoxib is in an amount of about 5 mg to about 40 mg per dose.

44. The combination of claim 40 wherein D₉₀ of the valdecoxib particles is less than about 75 μm.

45. The combination of claim 40 wherein the valdecoxib particles have a weight average particle size of about 1 to about 10 μm.

46. The combination of claim 40 that is a tablet wherein the excipients comprise one or more diluents in an amount of about 5% to about 99%, one or more disintegrants in an amount of about 0.2% to about 30%, one or more binding agents in an amount of about 0.5% to about 25%, and one or more lubricants in an amount of about 0.1% to about 10%, by weight of the composition.

47. The combination of any of claims 1-4 wherein the cyclooxygenase-2 inhibitor is formulated as solid particulate celecoxib having an average particle size of about 100 nm to about 800 nm.

48. The combination of claim 1 wherein the formulation has one or more orally deliverable dose units, each comprising a vasomodulator and a first fraction of celecoxib in an amount of about 10 mg to about 400 mg, the first fraction being in solution in a pharmaceutically acceptable solvent and/or present in immediate-release solid particles having a D₉₀ particle size less than about 1 μm; and a second fraction of celecoxib in an amount of about 10 mg to about 400 mg, the second fraction being present in solid particles having a D₉₀ particle size greater than about 25μm and/or in controlled-release, slow-release, programmed-release, timed-release, pulse-release, sustained-release or extended-release particles; wherein the first fraction and the second fraction of celecoxib are present in a ratio of about 10:1 to about 1:10 in the composition.

49. The combination of claim 48 wherein the first fraction of celecoxib is present in immediate-release solid particles having a D₉₀ particle size less than about 1 μm.

50. The combination of claim 1 wherein the vasomodulator is selected from the group consisting of a rennin-angiotensin system antagonist agent, a nitrovasodilator agent, a direct vasodilator agent, a calcium channel blocking drug, a phosphodiesterase inhibitor agent, a sympathomimetic agent, a sympatholytic agent, and nitric oxide synthase inhibitor.

51. The combination of claim 50 wherein the vasomodulator is a rennin-angiotensin system antagonist agent.

52. The combination of claim 51 wherein the rennin-angiotensin system antagonist agent is a compound selected from the group consisting of captopril, enalapril, enalaprilal, quinapril, lisinopril, ramipril, and losartan.

53. The combination of claim 52 wherein the rennin-angiotensin system antagonist agent is captopril.

54. The combination of claim 52 wherein the rennin-angiotensin system antagonist agent is enalapril.

55. The combination of claim 52 wherein the rennin-angiotensin system antagonist agent is quinapril.

56. The combination of claim 52 wherein the rennin-angiotensin system antagonist agent is lisinopril.

57. The combination of claim 52 wherein the rennin-angiotensin system antagonist agent is ramipril.

58. The combination of claim 52 wherein the rennin-angiotensin system antagonist agent is losartan.

59. The combination of claim 51 wherein the vasomodulator is a nitrovasodilator agent.

60. The combination of claim 59 wherein the nitrovasodilator agent is selected from the group consisting of nitroglycerin, isosobide dinitrate, and nitroprusside.

61. The combination of claim 60 wherein the nitrovasodilator agent is nitroglycerin.

62. The combination of claim 60 wherein the nitrovasodilator agent is isosobide dinitrate.

63. The combination of claim 60 wherein the nitrovasodilator agent is nitroprusside.

64. The combination of claim 50 wherein the vasomodulator is a direct vasodilator agent.

65. The combination of claim 64 wherein the direct vasodilator agent is selected from the group consisting of hydralazine, nicorandil, minoxidil, and diazoxide.

66. The combination of claim 65 wherein the direct vasodilator agent is hydralazine.

67. The combination of claim 65 wherein the direct vasodilator agent is nicorandil.

68. The combination of claim 65 wherein the direct vasodilator agent is minoxidil.

69. The combination of claim 65 wherein the direct vasodilator agent is diazoxide.

70. The combination of claim 50 wherein the vasomodulator is a calcium channel blocking drug.

71. The combination of claim 50 wherein the calcium channel blocking drug is selected from the group consisting of nifedipine, amlodipine, and felodipine.

72. The combination of claim 71 wherein the calcium channel blocking drug is nifedipine.

73. The combination of claim 71 wherein the calcium channel blocking drug is amlodipine.

74. The combination of claim 71 wherein the calcium channel blocking drug is felodipine.

75. The combination of claim 50 wherein the vasomodulator is a phosphodiesterase inhibitor agent.

76. The combination of claim 75 wherein the phosphodiesterase inhibitor agent is selected from the group consisting of amrinone, milrinone, and vesnarinone.

77. The combination of claim 76 wherein the phosphodiesterase inhibitor agent is amrinone.

78. The combination of claim 76 wherein the phosphodiesterase inhibitor agent is milrinone.

79. The combination of claim 76 wherein the phosphodiesterase inhibitor agent is vesnarinone.

80. The combination of claim 70 wherein the vasomodulator is a sympathomimetic agent.

81. The combination of claim 80 wherein the sympathomimetic agent is dobutamine.

82. The combination of claim 80 wherein the sympathomimetic agent is dopamine.

83. The combination of claim 50 wherein the vasomodulator is a sympatholytic agent.

84. The combination of claim 83 wherein the sympatholytic agent is selected from the group consisting of prazosin, phentolamine, labetalol, carvedilol, and bucindolol.

85. The combination of claim 50 wherein the sympatholytic agent is prazosin.

86. The combination of claim 84 wherein the sympatholytic agent is phentolamine.

87. The combination of claim 84 wherein the sympatholytic agent is labetalol.

88. The combination of claim 84 wherein the sympatholytic agent is carvedilol.

89. The combination of claim 84 wherein the sympatholytic agent is bucindolol.

90. The combination of claim 120 wherein the vasomodulator is a nitric oxide synthase inhibitor.

91. The combination according to claim 90, wherein the nitric oxide synthase inhibitor is an inducible nitric oxide synthase inhibitor.

92. An orally deliverable pharmaceutical composition comprising (a) a selective cyclooxygenase-2 inhibitory drug in a form providing rapid onset of therapeutic effect, and (b) a xanthine compound, wherein the selective cyclooxygenase-2 inhibitory drug and the xanthine compound are present in absolute and relative amounts effective to treat, prevent, ameliorate or delay headache pain.

93. The composition of claim 92 wherein the selective cyclooxygenase-2 inhibitory drug is in solid particulate form having a weight average particle size of about 0.1 to about 5 micrometers.

94. The composition of claim 93 wherein the selective cyclooxygenase-2 inhibitory drug has a weight average particle size of about 0.5 to about 3 micrometers.

95. The composition of claim 93 wherein the selective cyclooxygenase-2 inhibitory drug is at least partly in amorphous form.

96. The composition of claim 93 that is formulated as a discrete solid dosage form.

97. The composition of claim 93 that is formulated as a suspension in a pharmaceutically acceptable liquid diluent.

98. The composition of claim 93 that is formulated as a powder suitable for dilution in an aqueous diluent to form a suspension.

99. The composition of claim 93 wherein the selective cyclooxygenase-2 inhibitory drug is at least partly in dissolved or solubilized form in a pharmaceutically acceptable solvent liquid.

100. The composition of claim 99 wherein the solvent liquid comprises at least one solvent selected from glycols and glycol ethers.

101. The composition of claim 99 wherein the solvent liquid comprises polyethylene glycol.

102. The composition of claim 99 that is self-emulsifiable in simulated gastric fluid.

103. The composition of claim 99 wherein the selective cyclooxygenase-2 inhibitory drug is substantially all in dissolved or solubilized form.

104. The composition of claim 99 that is formulated as an imbibable liquid.

105. The composition of claim 99 that is formulated as a discrete dosage form encapsulated in a wall that releases the composition in the gastrointestinal tract.

106. A method of treatment, prevention, inhibition, amelioration or delay of pain in a subject, the method consisteing essentially of administering to the subject an amount of a high energy form of a selective cyclooxygenase-2 inhibitor, and administering to the subject and amount of a vasomodulator compound which together comprise a therapeutically effective amount of the selective cyclooxygenase-2 inhibitor and the vasomodulator.

107. The method of claim 106 comprising orally administering to a mammalian subject in need of analgesia an effective pain-relieving amount of a pharmaceutical formulation of claim 1 formulated in such a way as to provide, when tested in fasting humans in accordance with standard pharmacokinetic practice, a blood plasma concentration profile of celecoxib in which a concentration of about 250 ng/ml is attained not later than about 30 minutes after oral administration.

108. The method of claim 107 wherein a blood plasma concentration of celecoxib of about 250 ng/ml is attained not later than about 15 minutes after oral administration.

109. The method of claim 106 wherein the vasomodulator is a xanthine compound.

110. The method of claim 109 wherein the xanthine compound is caffeine.

111. The method according to claim 106 wherein the pain is selected from the group consisting of migraine headache pain, cluster headache pain, chronic headache pain, substance-induced headache pain, tension or stress related headache pain, sinus headache pain, pain resulting from anesthesia, headache pain associated with increased intracranial pressure, headache pain associated with decreased intracranial pressure, headache pain resulting from giant cell arteritis, and headache pain resulting from lumbar puncture.

112. The method of claim 106 wherein the selective cyclooxygenase-2 inhibitor is a compound selected from the group consisting of celecoxib, rofecoxib, valdecoxib, deracoxib, etoricoxib, 2-(3,4-Difluorophenyl)-4-(3-hydroxy-3-methylbutoxy)-5-[4-(methylsulfonyl)phenyl]-3(2H)-pyridazinone, parecoxib, and meloxicam.

113. The method of claim 106 wherein the selective cyclooxygenase-2 inhibitor is selected from compounds of Formula II: 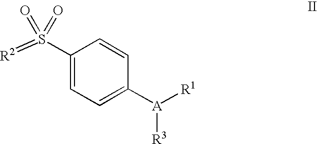
wherein A is selected from the group consisting of partially unsaturated or unsaturated heterocyclyl and partially unsaturated or unsaturated carbocyclic rings;
wherein R¹ is selected from the group consisting of heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;
wherein R² is selected from the group consisting of methyl or amino; and
wherein R³ is selected from the group consisting of a radical selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl; or a pharmaceutically acceptable salt thereof.

114. The method of any of claim 106 wherein the selective cyclooxygenase-2 inhibitor is selected from compounds of Formula I: 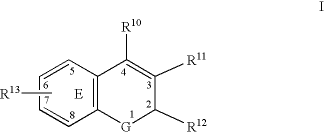
wherein G is selected from the group consisting of O or S or NR^{a}; wherein R^{a} is alkyl;
wherein R¹⁰ is selected from the group consisting of H and aryl
wherein R¹¹ is selected from the group consisting of carboxyl, aminocarbonyl, alkylsulfonylaminocarbonyl and alkoxycarbonyl;
wherein R¹² is selected from the group consisting of haloalkyl, alkyl, aralkyl, cycloalkyl and aryl optionally substituted with one or more radicals selected from alkylthio, nitro and alkylsulfonyl; and
wherein R¹³ is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;
or wherein R¹³ together with ring E forms a naphthyl radical;
or a pharmaceutically acceptable salt or isomer or prodrug thereof.

115. The method of claim 106 wherein the selective cyclooxygenase-2 inhibitor is selected from compounds of Formula III: 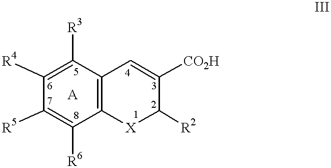
wherein X is O or S;
R² is lower haloalkyl;
R³is selected from the group consisting of hydrido and halo;
R⁴ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, a 5-membered nitrogen containing heterocyclosulfonyl, and a 6-membered nitrogen containing heterocyclosulfonyl;
R⁵ is selected from the group consisting of hydrido, lower alkyl, halo, lower alkoxy, and aryl; and
R⁶ is selected from the group consisting of hydrido, halo, lower alkyl, lower alkoxy, and aryl.
or a pharmaceutically acceptable salt, or isomer, or prodrug thereof.

116. The method of claim 106 wherein the selective cyclooxygenase-2 inhibitor is selected from compounds of Formula IV: 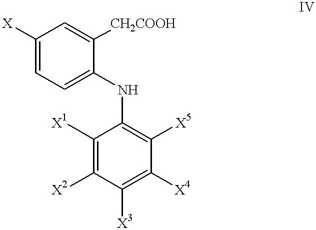
wherein X is methyl or ethyl;
X¹ is chloro or fluoro;
X² is hydrido or fluoro;
X³ is hydrido, fluoro, chloro, methyl, ethyl, methoxy, ethoxy, or hydroxy;
X⁴ is hydrido or fluoro; and
X⁵ is chloro, fluoro, trifluoromethyl or methyl;
pharmaceutically acceptable salts thereof; and pharmaceutically acceptable prodrug esters thereof.

117. The method according to claim 106 wherein the selective cyclooxygenase-2 inhibitor and the vasomodulator are administered combined in a single dosage form.

118. The method according to claim 110 wherein a single tablet, pill or capsule of the single dosage form comprises a selective cyclooxygenase-2 inhibitor in an amount of from about 0.5 mg to about 500 mg, and caffeine in an amount of about 10 to 400 mg.

119. The method according to claim 110 wherein a single tablet, pill or capsule of the single dosage form comprises a selective cyclooxygenase-2 inhibitor in an amount of from about 1 mg to about 200 mg, and caffeine in an amount of about 55 to 100 mg.

120. A method for treatment, prevention, or amelioration of headache symptoms, or for delay of onset thereof, comprising administering to a patient suffering from or subject to headache pain a combination comprising a selective cyclooxygenase-2 inhibitor and a vasomodulator, the selective cyclooxygenase-2 inhibitor and vasomodulator each being administered in an amount effective to contribute to the prevention, amelioration or delay or headache pain, the IC₅₀ of the combination for binding of 5TH₁ receptors being at least about 250 nM.

121. The method according to claim 120 wherein the combination for binding of 5HT₁ receptors is at least about 500 nM.

122. The method according to claim 120 wherein the combination for binding of 5HT₁ receptors is at least about 750 nM.

123. The method according to claim 120 wherein the combination for binding of 5HT₁ receptors is at least about 1000 nM.

124. The method according to claim 120 wherein the combination for binding of 5HT₁ receptors is at least about 5000 nM.

125. The method according to claim 120 wherein the combination for binding of 5HT₁ receptors is at least about 10,000 nM.
